SEQUENCE LISTING (72 sequences)

SEQ ID NO: 1 - protein, 996 aa, Arabidopsis thaliana
MEPTEKPSTKPSSRTLPRDTRGSLEVFNPSTQLTRPDNPVFRPEPPAWQNLSDPRGTSPQPRPQQEPAPSNPVRSDQEIAVTTSWMALKDPSPETISKKTITAEKPQKSAVAAEQRAAEWGLVLKTDTKTGKPQGVGVRNSGGTENDPNGKKTTSQRNSQNSCRSSGEMSDGDVPGGRSGIPRVSEDLKDALSTFQQTFVVSDATKPDYPIMYASAGFFNMTGYTSKEVVGRNCRFLQGSGTDADELAKIRETLAAGNNYCGRILNYKKDGTSFWNLLTIAPIKDESGKVLKFIGMQVEVSKHTEGAKEKALRPNGLPESLIRYDARQKDMATNSVTELVEAVKRPRALSESTNLHPFMTKSESDELPKKPARRMSENVVPSGRRNSGGGRRNSMQRINEIPEKKSRKSSLSFMGIKKKSESLDESIDDGFIEYGEEDDEISDRDERPESVDDKVRQKEMRKGIDLATTLERIEKNFVITDPRLPDNPIIFASDSFLELTEYSREEILGRNCRFLQGPETDLTTVKKIRNAIDNQTEVTVQLINYTKSGKKFWNIFHLQPMRDQKGEVQYFIGVQLDGSKHVEPVRNVIEETAVKEGEDLVKKTAVNIDEAVRELPDANMTPEDLWANHSKVVHCKPHRKDSPPWIAIQKVLESGEPIGLKHFKPVKPLGSGDTGSVHLVELVGTDQLFAMKAMDKAVMLNRNKVHRARAEREILDLLDHPFLPALYASFQTKTHICLITDYYPGGELFMLLDRQPRKVLKEDAVRFYAAQVVVALEYLHCQGIIYRDLKPENVLIQGNGDISLSDFDLSCLTSCKPQLLIPSIDEKKKKKQQKSQQTPIFMAEPMRASNSFVGTEEYIAPEIISGAGHTSAVDWWALGILMYEMLYGYTPFRGKTRQKTFTNVLQKDLKFPASIPASLQVKQLIFRLLQRDPKKRLGCFEGANEVKQHSFFKGINWALIRCTNPPELETPIFSGEAENGEKVVDPELEDLQTNVF

SEQ ID NO: 2 - protein, 138 aa, Arabidopsis thaliana
ESVDDKVRQKEMRKGIDLATTLERIEKNFVITDPRLPDNPIIFASDSFLELTEYSREEILGRNCRFLQGPETDLTTVKKIRNAIDNQTEVTVQLINYTKSGKKFWNIFHLQPMRDQKGEVQYFIGVQLDGSKHVEPVR

SEQ ID NO: 3 - protein, 915 aa, Arabidopsis thaliana
MERPRAPPSPLNDAESLSERRSLEIFNPSSGKETHGSTSSSSKPPLDGNNKGSSSKWMEFQDSAKITERTAEWGLSAVKPDSGDDGISFKLSSEVERSKNMSRRSSEESTSSESGAFPRVSQELKTALSTLQQTFVVSDATQPHCPIVYASSGFFTMTGYSSKEIVGRNCRFLQGPDTDKNEVAKIRDCVKNGKSYCGRLLNYKKDGTPFWNLLTVTPIKDDQGNTIKFIGMQVEVSKYTEGVNDKALRPNGLSKSLIRYDARQKEKALDSITEVVQTIRHRKSQVQESVSNDTMVKPDSSTTPTPGRQTRQSDEASKSFRTPGRVSTPTGSKLKSSNNRHEDLLRMEPEELMLSTEVIGQRDSWDLSDRERDIRQGIDLATTLERIEKNFVISDPRLPDNPIIFASDSFLELTEYSREEILGRNCRFLQGPETDQATVQKIRDAIRDQREITVQLINYTKSGKKFWNLFHLQPMRDQKGELQYFIGVQLDGSDHVEPLQNRLSERTEMQSSKLVKATATNVDEAVRELPDANTRPEDLWAAHSKPVYPLPHNKESTSWKAIKKIQASGETVGLHHFKPIKPLGSGDTGSVHLVELKGTGELYAMKAMEKTMMLNRNKAHRACIEREIISLLDHPFLPTLYASFQTSTHVCLITDFCPGGELFALLDRQPMKILTEDSARFYAAEVVIGLEYLHCLGIVYRDLKPENILLKKDGHIVLADFDLSFMTTCTPQLIIPAAPSKRRRSKSQPLPTFVAEPSTQSNSFVGTEEYIAPEIITGAGHTSAIDWWALGILLYEMLYGRTPFRGKNRQKTFANILHKDLTFPSSIPVSLVGRQLINTLLNRDPSSRLGSKGGANEIKQHAFFRGINWPLIRGMSPPPLDAPLSIIEKDPNAKDIKWEDDGVLVNSTDLDIDLF

SEQ ID NO: 4 - protein, 138 aa, Arabidopsis thaliana
DSWDLSDRERDIRQGIDLATTLERIEKNFVISDPRLPDNPIIFASDSFLELTEYSREEILGRNCRFLQGPETDQATVQKIRDAIRDQREITVQLINYTKSGKKFWNLFHLQPMRDQKGELQYFIGVQLDGSDHVEPLQ

SEQ ID NO: 5 - protein, 750 aa, Chlamydomonas reinhardtii
MAGVPAPASQLTKVLAGLRHTFVVADATLPDCPLVYASEGFYAMTGYGPDEVLGHNCRFLQGEGTDPKEVQKIRDAIKKGEACSVRLLNYRKDGTPFWNLLTVTPIKTPDGRVSKFVGVQVDVTSKTEGKALADNSGVPLLVKYDHRLRDNVARTIVDDVTIAVEKAEGVEPGQASAVAAAAPLGAKGPRGTAPKSFPRVALDLATTVERIQQNFCISDPTLPDCPIVFASDAFLELTGYSREEVLGRNCRFLQGAGTDRGTVDQIRAAIKEGSELTVRILNYTKAGKAFWNMFTLAPMRDQDGHARFFVGVQVDVTAQSTSPDKAPVWNKTPEEEVAKAKMGAEAASLISSALQGMAAPTTANPWAAISGVIMRRKPHKADDKAYQALLQLQERDGKMKLMHFRRVKQLGAGDVGLVDLVQLQGSELKFAMKTLDKFEMQERNKVARVLTESAILAAVDHPFLATLYCTIQTDTHLHFVMEYCDGGELYGLLNSQPKKRLKEEHVRFYASEVLTALQYLHLLGYVYRDLKPENILLHHTGHVLLTDFDLSYSKGSTTPRIEKIGGAGAAGGSAPKSPKKSSSKSGGSSSGSALQLENYLLLAEPSARANSFVGTEEYLAPEVINAAGHGPAAVDWWSLGILIFELLYGTTPFRGARRDETFENIIKSPLKFPSKPAVSEECRDLIEKLLVKDVGARLGSRTGANEIKSHPWFKGINWALLRHQQPPYVPRRASKAAGGSSTGGAAFDNY

SEQ ID NO: 6 - protein, 118 aa, Chlamydomonas reinhardtii
AGLRHTFVVADATLPDCPLVYASEGFYAMTGYGPDEVLGHNCRFLQGEGTDPKEVQKIRDAIKKGEACSVRLLNYRKDGTPFWNLLTVTPIKTPDGRVSKFVGVQVDVTSKTEGKALA

SEQ ID NO: 7 - protein, 186 aa, Neurospora crassa
MSHTVNSSTMNPWEVEAYQQYHYDPRTAPTANPLFFHTLYAPGGYDIMGYLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETE

SEQ ID NO: 8 - protein, 150 aa, Neurospora crassa
HTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETE

SEQ ID NO: 9 - protein, 348 aa, Vaucheria frigida
MNGLTPPLMFCSRSDDPSSTSNINLDDVFADVFFNSNGELLDIDEIDDFGDNTCPKSSMSVDDDASSQVFQGHLFGNALSSIALSDSGDLSTGIYESQGNASRGKSLRTKSSGSISSELTEAQKVERRERNREHAKRSRVRKKFLLESLQQSVNELNHENNCLKESIREHLGPRGDSLIAQCSPEADTLLTDNPSKANRILEDPDYSLVKALQMAQQNFVITDASLPDNPIVYASRGFLTLTGYSLDQILGRNCRFLQGPETDPRAVDKIRNAITKGVDTSVCLLNYRQDGTTFWNLFFVAGLRDSKGNIVNYVGVQSKVSEDYAKLLVNEQNIEYKGVRTSNMLRRK

SEQ ID NO: 10 - protein, 145 aa, Vaucheria frigida
PDYSLVKALQMAQQNFVITDASLPDNPIVYASRGFLTLTGYSLDQILGRNCRFLQGPETDPRAVDKIRNAITKGVDTSVCLLNYRQDGTTFWNLFFVAGLRDSKGNIVNYVGVQSKVSEDYAKLLVNEQNIEYKGVRTSNMLRRK

SEQ ID NO: 11 - protein, 228 aa, Nannochloropsis gaditana
MTEEQKVERRERNREHAKRSRVRKKFLLESLQKSVNALQEENDKLRGAIRSHLKEGADDLLKTCEVEVDESILASDPCSATKILDDPDYTLVKALQTAQQNFVITDPTLPDNPIVYASGGFLSLTGYQMDQILGRNCRFLQGPDTDPAAVDKIRRAIEDGTDGSVCLLNYRADGSTFWNQFFIAALRGADGNIVNYVGVQCKVSEEYASEVLKKEATSSTVAEASSKR

SEQ ID NO: 12 - protein, 142 aa, Nannochloropsis gaditana
PDYTLVKALQTAQQNFVITDPTLPDNPIVYASGGFLSLTGYQMDQILGRNCRFLQGPDTDPAAVDKIRRAIEDGTDGSVCLLNYRADGSTFWNQFFIAALRGADGNIVNYVGVQCKVSEEYASEVLKKEATSSTVAEASSKR

SEQ ID NO: 13 - protein, 312 aa, Ochromonas danica
MTSKQQLPPPPIFGVLGDEKQVARNGIISLVDIFDDFLFSGDRNQPSNTASSSSHAQESESVGKDEENDYDSNDDEGDSDDGKRRKRSRTLPRNMTEEQKIERRERNREHAKRSRVRKKFLLESLQHSVRALEEENEKLRNAIRENLQGEAEQLLTRCSCGGPSVIASDPNTATRTLDDPDYSLVKALQTAQQNFVISDPSIPDNPIVYASQGFLTLTGYALSEVLGRNCRFLQGPETDPKAVEKVRKGLERGEDTTVVLLNYRKDGSTFWNQLFIAALRDGEGNVVNYLGVQCKVSEDYAKAFLKNEENEK

SEQ ID NO: 14 - protein, 133 aa, Ochromonas danica
PDYSLVKALQTAQQNFVISDPSIPDNPIVYASQGFLTLTGYALSEVLGRNCRFLQGPETDPKAVEKVRKGLERGEDTTVVLLNYRKDGSTFWNQLFIAALRDGEGNVVNYLGVQCKVSEDYAKAFLKNEENEK

SEQ ID NO: 15 - DNA, 36 bp, Artificial
  Primer imFGFR1_XhoI_Kozak_F
cagagctcgagaccatgtggagctggaagtgcctcc

SEQ ID NO: 16 - DNA, 32 bp, Artificial
  Primer imFGFR1_BamHI_R
cagaaggatcctcagcggcgtttgagtccgcc

SEQ ID NO: 17 - DNA, 28 bp, Artificial
  Primer imFGFR1_inverse_R
tgagaccggtctcgacgcgccgtttgag

SEQ ID NO: 18 - DNA, 29 bp, Artificial
  Primer imFGFR1_inverse1_F
caagaccggtggatccggagtcgactatc

SEQ ID NO: 19 - DNA, 32 bp, Artificial
  Primer imFGFR1_inverse2_F
caagaccggtaaactggaagtcgagggagtgc

SEQ ID NO: 20 - DNA, 32 bp, Artificial
  Primer FKBP_AgeI_F
gatcaccggtaaactggaagtcgagggagtgc

SEQ ID NO: 21 - DNA, 34 bp, Artificial
  Primer FKBP_XmaI_R
gatccccgggaccgccagattccagttttagaag

SEQ ID NO: 22 - DNA, 41 bp, Artificial
  Primer AtPH1-LOV2_AgeI_F
gatcaccggtgaaagcgttgatgataaggtcagacagaagg

SEQ ID NO: 23 - DNA, 30 bp, Artificial
  Primer AtPH1-LOV2_XmaI_R
gatccccgggccgcacgggctcaacgtgct

SEQ ID NO: 24 - DNA, 40 bp, Artificial
  Primer AtPH2-LOV2_AgeI_F
gatcaccggtgattcttgggatctgagtgatagggaaagg

SEQ ID NO: 25 - DNA, 35 bp, Artificial
  Primer AtPH2-LOV2_XmaI_R
gatccccgggctggagtggctcgacatgatctgac

SEQ ID NO: 26 - DNA, 38 bp, Artificial
  Primer CrPH1-LOV1_AgeI_F
gatcaccggtgcaggactcagacatacatttgtggtgg

SEQ ID NO: 27 - DNA, 32 bp, Artificial
  Primer CrPH1-LOV1_XmaI_R
gatccccgggggccagggctttcccttcagtc

SEQ ID NO: 28 - DNA, 32 bp, Artificial
  Primer NcVV-LOV_XmaI_F
gatccccgggcacactctctacgccccaggcg

SEQ ID NO: 29 - DNA, 37 bp, Artificial
  Primer NcVV-LOV_XmaI_R
gatccccgggttcggtttcgcactgaaaacccatgct

SEQ ID NO: 30 - DNA, 32 bp, Artificial
  Primer VfAU1-LOV_AgeI_F
gatcaccggtcctgactacagtctcgtgaagg

SEQ ID NO: 31 - DNA, 33 bp, Artificial
  Primer VfAU1-LOV_XmaI_R
gatccccgggctttctgcgcagcatgttactgg

SEQ ID NO: 32 - DNA, 45 bp, Artificial
  Primer Opto-mFGFR1_YY271/2FF_F
gagacattcatcatatcgacttcttcaagaaaaccaccaacggcc

SEQ ID NO: 33 - DNA, 45 bp, Artificial
  Primer Opto-mFGFR1_YY271/2FF_R
ggccgttggtggttttcttgaagaagtcgatatgatgaatgtctc

SEQ ID NO: 34 - DNA, 40 bp, Artificial
  Primer Opto-mFGFR1_R195E_F
tacaggcccgggagcctcctgggctggagtactgctataa

SEQ ID NO: 35 - DNA, 40 bp, Artificial
  Primer Opto-mFGFR1_R195E_R
ttatagcagtactccagcccaggaggctcccgggcctgta

SEQ ID NO: 36 - DNA, 32 bp, Artificial
  Primer Opto-mFGFR1_I472V_F
ctcccagacaaccctgtcgtctacgccagtag

SEQ ID NO: 37 - DNA, 32 bp, Artificial
  Primer Opto-mFGFR1_I472V_R
ctactggcgtagacgacagggttgtctgggag

SEQ ID NO: 38 - DNA, 32 bp, Artificial
  Primer VfAU1-LOV_BgIII_F
ctttagatctcctgactacagtctcgtgaagg

SEQ ID NO: 39 - DNA, 32 bp, Artificial
  Primer VfAU1-LOV_EcoRI_R
ctttgaattcctttctgcgcagcatgttactg

SEQ ID NO: 40 - DNA, 31 bp, Artificial
  Primer mFGFR1_inverse_R
gatccaccggtgacgtcgaggcgctggctgg

SEQ ID NO: 41 - DNA, 35 bp, Artificial
  Primer Opto-mFGFR1_inverse_F
gatccaccggtggacctgactacagtctcgtgaag

SEQ ID NO: 42 - DNA, 35 bp, Artificial
  Primer imFGFR1_inverse_F
gatccaccggtggaaaactggaagtcgagggagtg

SEQ ID NO: 43 - DNA, 37 bp, Artificial
  Primer hEGFR_AgeI_AscI_F
gatcaccggtggcgcgcccgaaggcgccacatcgttc

SEQ ID NO: 44 - DNA, 34 bp, Artificial
  Primer hEGFR_ICD_BspEI_R
gatctccggatgctccaataaattcactgctttg

SEQ ID NO: 45 - DNA, 31 bp, Artificial
  Primer hRET_ICD_AgeI_F
gatcaccggtcactgctaccacaagtttgcc

SEQ ID NO: 46 - DNA, 28 bp, Artificial
  Primer hRET_ICD_AgeI_R
gatcaccggtgaatctagtaaatgcatg

SEQ ID NO: 47 - DNA, 33 bp, Artificial
  Primer LNGFR_ECD_NotI_F
gatcgcggccgcaccatgggggcaggtgccacc

SEQ ID NO: 48 - DNA, 35 bp, Artificial
  Primer LNGFR_ECD_AscI_R
gatcggcgcgccccctcttgaaggctatgtaggcc

SEQ ID NO: 49 - DNA, 414 bp, Artificial
  Codon optimized AtPH1-LOV2
gaaagcgttgatgataaggtcagacagaaggaaatgagaaagggaatcgatctcgcaacaacactcgaaagaatagaaaagaactttgtgattactgaccctaggctccccgataatcccataatcttcgcttcagacagtttcctggagctgacagagtatagccgggaagagatcctgggtagaaattgcagattcctgcagggacccgagacagacctgaccaccgtgaagaagattcgcaatgctatcgataatcaaaccgaggttaccgtgcaactgataaactacactaaaagcggcaagaagttctggaacattttccacctgcagcctatgcgggaccagaagggtgaggtccaatatttcatcggggtgcagctggatggcagcaagcacgttgagcccgtgcgg

SEQ ID NO: 50 - DNA, 414 bp, Artificial
  Codon optimized AtPH2-LOV2
gattcttgggatctgagtgatagggaaagggatattagacagggaatagacctcgccaccaccctggaaagaattgaaaagaatttcgtgatcagcgaccctagactgcccgacaatccaatcattttcgcctctgactcttttctggagctgaccgaatactcacgcgaagaaatcctgggaaggaactgtaggttcctgcaaggacccgaaaccgaccaggccactgtccagaagattcgcgatgccatccgcgaccagcgggaaattaccgttcaactgatcaactataccaaatctggtaagaagttttggaacctgttccacctccagcctatgcgggaccaaaagggcgaactgcaatatttcatcggggtgcagctggacgggtcagatcatgtcgagccactccag

SEQ ID NO: 51 - DNA, 354 bp, Artificial
  Codon optimized CrPH-LOV1
gcaggactcagacatacatttgtggtggctgatgcaacactccctgattgcccactggtctatgcaagtgagggcttctacgcaatgaccggatatggacctgacgaagtgctgggtcacaactgtaggtttctgcagggtgagggaactgaccccaaggaagtgcagaaaattcgcgacgccatcaagaagggtgaggcttgtagtgtgcgcctcctgaactatcggaaggacggcactcccttctggaacctgctgacagtcaccccaattaaaacccctgatggccgcgtgtccaagtttgtcggcgtgcaggtggatgttacctccaagactgaagggaaagccctggcc

SEQ ID NO: 52 - DNA, 450 bp, Artificial
  Codon optimized NcVV-LOV
cacactctctacgccccaggcgggtacgatattatgggctggctgatccagatcatgaacaggcccaatccccaggtcgagctgggacccgtggatacttcatgtgcactgatactgtgcgacctgaagcagaaggatacacctatagtttacgcttcagaagcctttctgtacatgacagggtattctaacgccgaggtgctggggaggaactgtaggttcctccagagtcccgatggtatggtgaaacctaagagtactcgcaaatatgtggatagcaatactattaacaccatgaggaaagccatcgacagaaacgcagaagttcaggtggaagtggtgaactttaagaagaacggccagcggttcgtgaactttctcacaatgattccagtgcgggacgaaaccggggagtaccggtacagcatgggttttcagtgcgaaaccgaa

SEQ ID NO: 53 - DNA, 435 bp, Artificial
  Codon optimized VfAU1-LOV
cctgactacagtctcgtgaaggctctgcaaatggcacaacagaattttgtcattacagacgcctccctcccagacaaccctatcgtctacgccagtagagggtttctgacactgacaggctattctctcgaccagatcctgggcaggaactgcaggtttctgcaagggccagaaacagacccaagagctgtggataagatcaggaatgccatcaccaaaggcgttgataccagtgtctgtctgctgaattatagacaggatggcacaaccttctggaatctcttcttcgtggctggactcagagattctaagggcaatattgtcaactacgtcggagtgcagtcaaaggtgagcgaagattatgccaagctgctggtcaacgagcagaacattgagtacaaaggtgtgcgcaccagtaacatgctgcgcagaaag

SEQ ID NO: 54 - DNA, 426 bp, Artificial
  Codon optimized NgPA-LOV
ccagattatacactcgttaaagcactgcaaactgctcagcagaattttgtgatcaccgaccctactctgccagacaaccccattgtctatgcttcaggaggatttctcagtctcacaggttaccagatggatcagatcctgggaagaaattgcagatttctgcaaggacctgatactgacccagctgccgtggacaagatcagaagggctatcgaagatggtacagacggcagtgtctgtctgctgaactacagagcagatggatctaccttttggaatcaattcttcattgctgctctcagaggcgctgacggaaatatcgtcaactatgtcggagtgcagtgtaaagtgtcagaggagtatgcttcagaagtcctcaagaaggaggctacttcatccactgtggctgaagcaagtagcaaaaga

SEQ ID NO: 55 - DNA, 399 bp, Artificial
  Codon optimized OdPA1-LOV
cctgactacagtctggttaaagcactccaaacagcacagcagaatttcgttatctctgaccctagcattcctgataatcccattgtgtatgctagtcagggatttctgacactcaccggatacgcactgagcgaggttctcggacggaactgccggttcctccaaggaccagaaacagaccctaaagccgtcgagaaagtgagaaagggtctggagagaggtgaagataccaccgtggtgctcctgaattataggaaagatggaagcaccttctggaaccaactgttcattgctgccctgcgggatggtgagggcaatgtggttaactacctcggagttcagtgcaaagtctccgaggactacgccaaagcctttctgaagaatgaagagaacgagaaa

SEQ ID NO: 56 - protein, 575 aa, Artificial
  miFGFR1
MGSSKSKPKDPSQRLDMKSGTKKSDFHSQMAVHKLAKSIPLRRQVTVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSIPLDQYSPSFPDTRSSTCSSGEDSVFSHEPLPEEPCLPRHPTQLANSGLKRRVETGKLEVEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDYPYDVPDYALD

SEQ ID NO: 57 - protein, 460 aa, Artificial
  miFGFR1-(Delta)FKBP
MGSSKSKPKDPSQRLDMKSGTKKSDFHSQMAVHKLAKSIPLRRQVTVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSIPLDQYSPSFPDTRSSTCSSGEDSVFSHEPLPEEPCLPRHPTQLANSGLKRRVETGGSGVDYPYDVPDYALD

SEQ ID NO: 58 - protein, 607 aa, Artificial
  mFGFR1-VfAU1-LOV
MGSSKSKPKDPSQRLDMKSGTKKSDFHSQMAVHKLAKSIPLRRQVTVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSIPLDQYSPSFPDTRSSTCSSGEDSVFSHEPLPEEPCLPRHPTQLANSGLKRRVETGPDYSLVKALQMAQQNFVITDASLPDNPIVYASRGFLTLTGYSLDQILGRNCRFLQGPETDPRAVDKIRNAITKGVDTSVCLLNYRQDGTTFWNLFFVAGLRDSKGNIVNYVGVQSKVSEDYAKLLVNEQNIEYKGVRTSNMLRRKTGGSGVDYPYDVPDYALD

SEQ ID NO: 59 - protein, 985 aa, Artificial [sequence omitted]

SEQ ID NO: 60 - protein, 600 aa, Artificial
  hRET-VfAU1-LOV
MGSSKSKPKDPSQRLDVTGHCYHKFAHKPPISSAEMTFRRPAQAFPVSYSSSSARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRFTGGPDYSLVKALQMAQQNFVITDASLPDNPIVYASRGFLTLTGYSLDQILGRNCRFLQGPETDPRAVDKIRNAITKGVDTSVCLLNYRQDGTTFWNLFFVAGLRDSKGNIVNYVGVQSKVSEDYAKLLVNEQNIEYKGVRTSNMLRRKPGGSGVDYPYDVPDYALD

SEQ ID NO: 61 - DNA, 43 bp, Artificial
  SyCP1-PHY_F_XmaI
gatccccggggcaactactgttcaactgtctgatcaatctctg

SEQ ID NO: 62 - DNA, 37 bp, Artificial
  SyCP1-PHY _R_XmaI
gatccccgggttcttcagcttggcgcagaatcaggtt

SEQ ID NO: 63 - protein, 748 aa, Synechocystis PCC6803
MATTVQLSDQSLRQLETLAIHTAHLIQPHGLVVVLQEPDLTISQISANCTGILGRSPEDLLGRTLGEVFDSFQIDPIQSRLTAGQISSLNPSKLWARVMGDDFVIFDGVFHRNSDGLLVCELEPAYTSDNLPFLGFYHMANAALNRLRQQANLRDFYDVIVEEVRRMTGFDRVMLYRFDENNHGDVIAEDKRDDMEPYLGLHYPESDIPQPARRLFIHNPIRVIPDVYGVAVPLTPAVNPSTNRAVDLTESILRSAYHCHLTYLKNMGVGASLTISLIKDGHLWGLIACHHQTPKVIPFELRKACEFFGRVVFSNISAQEDTETFDYRVQLAEHEAVLLDKMTTAADFVEGLTNHPDRLLGLTGSQGAAICFGEKLILVGETPDEKAVQYLLQWLENREVQDVFFTSSLSQIYPDAVNFKSVASGLLAIPIARHNFLLWFRPEVLQTVNWGGDPNHAYEATQEDGKIELHPRQSFDLWKEIVRLQSLPWQSVEIQSALALKKAIVNLILRQAEELAQLARNLERSNADLKKFAYIASHDLQEPLNQVSNYVQLLEMRYSEALDEDAKDFIDFAVTGVSLMQTLIDDILTYAKVDTQYAQLTFTDVQEVVDKALANLKQRIEESGAEIEVGSMPAVMADQIQLMQVFQNLIANGIKFAGDKSPKIKIWGDRQEDAWVFAVQDNGIGIDPQFFERIFVIFQRLHTRDEYKGTGMGLAICKKIIEGHQGQIWLESNPGEGSTFYFSIPIGN

SEQ ID NO: 64 - protein, 513 aa, Synechocystis PCC6803
ATTVQLSDQSLRQLETLAIHTAHLIQPHGLVVVLQEPDLTISQISANCTGILGRSPEDLLGRTLGEVFDSFQIDPIQSRLTAGQISSLNPSKLWARVMGDDFVIFDGVFHRNSDGLLVCELEPAYTSDNLPFLGFYHMANAALNRLRQQANLRDFYDVIVEEVRRMTGFDRVMLYRFDENNHGDVIAEDKRDDMEPYLGLHYPESDIPQPARRLFIHNPIRVIPDVYGVAVPLTPAVNPSTNRAVDLTESILRSAYHCHLTYLKNMGVGASLTISLIKDGHLWGLIACHHQTPKVIPFELRKACEFFGRVVFSNISAQEDTETFDYRVQLAEHEAVLLDKMTTAADFVEGLTNHPDRLLGLTGSQGAAICFGEKLILVGETPDEKAVQYLLQWLENREVQDVFFTSSLSQIYPDAVNFKSVASGLLAIPIARHNFLLWFRPEVLQTVNWGGDPNHAYEATQEDGKIELHPRQSFDLWKEIVRLQSLPWQSVEIQSALALKKAIVNLILRQAEE

SEQ ID NO: 65 - DNA, 1539 bp, Artificial
  Codon-optimized SyCP1-PHY
gcaactactgttcaactgtctgatcaatctctgcgtcaactggaaactctggctatccacaccgcgcatctgatccagccgcacggtctggtagtcgtcctgcaagaaccggacctgaccatcagccagatctctgcgaactgtaccggtatcctgggccgtagcccggaagatctgctgggtcgtactctgggcgaggtattcgattcttttcagattgatccgatccagtctcgtctgaccgcaggtcagatttccagcctgaacccgtccaagctgtgggcgcgtgttatgggtgacgactttgttattttcgacggcgtatttcatcgtaactctgatggcctgctggtttgcgagctggagccggcctacactagcgacaacctgcctttcctgggtttctaccatatggcaaacgcggcactgaaccgtctgcgtcagcaagctaacctgcgcgacttctacgacgttatcgttgaggaagtgcgccgcatgacgggtttcgaccgcgtcatgctgtaccgttttgatgaaaacaaccacggtgacgtaatcgcggaggataagcgtgacgacatggagccgtatctgggtctgcactacccggaaagcgacattcctcagccggcacgtcgcctgttcattcacaacccgatccgtgttattccggacgtttacggcgttgctgttccgctgactccggccgttaatccgtctactaaccgtgcagttgacctgaccgaatccatcctgcgttccgcataccattgccacctgacctatctgaagaacatgggcgttggtgctagcctgacgatctctctgattaaagatggtcacctgtggggtctgatcgcttgccatcaccagaccccgaaagtaatccctttcgaactgcgtaaagcctgcgaattcttcggtcgtgtggtgttctctaatatctccgcgcaagaagacaccgagacttttgactaccgcgtacagctggcggagcatgaagcggttctgctggacaaaatgaccaccgcggcagacttcgtggagggcctgactaaccacccagaccgtctgctgggcctgaccggcagccaaggcgctgcgatttgtttcggcgagaaactgattctggtgggcgaaaccccagacgaaaaggcggtgcaatacctgctgcaatggctggagaatcgcgaagtgcaggacgttttcttcactagctctctgtctcagatctatccggatgcggttaacttcaaaagcgtggcgtccggcctgctggctatcccgatcgcccgtcataactttctgctgtggttccgcccggaggttctgcagaccgttaattggggtggtgatccgaatcacgcatacgaagcaacccaagaagatggtaagatcgaactgcatccgcgtcagtccttcgatctgtggaaagaaattgttcgcctgcagagcctgccgtggcagagcgttgagatccagtctgccctggctctgaagaaagcaatcgtgaacctgattctgcgccaagctgaagaa

SEQ ID NO: 66 - protein, 975 aa, Artificial
  redOpto-mFGFR1
MGSSKSKPKDPSQRLDMKSGTKKSDFHSQMAVHKLAKSIPLRRQVTVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSIPLDQYSPSFPDTRSSTCSSGEDSVFSHEPLPEEPCLPRHPTQLANSGLKRRVETGATTVQLSDQSLRQLETLAIHTAHLIQPHGLVVVLQEPDLTISQISANCTGILGRSPEDLLGRTLGEVFDSFQIDPIQSRLTAGQISSLNPSKLWARVMGDDFVIFDGVFHRNSDGLLVCELEPAYTSDNLPFLGFYHMANAALNRLRQQANLRDFYDVIVEEVRRMTGFDRVMLYRFDENNHGDVIAEDKRDDMEPYLGLHYPESDIPQPARRLFIHNPIRVIPDVYGVAVPLTPAVNPSTNRAVDLTESILRSAYHCHLTYLKNMGVGASLTISLIKDGHLWGLIACHHQTPKVIPFELRKACEFFGRVVFSNISAQEDTETFDYRVQLAEHEAVLLDKMTTAADFVEGLTNHPDRLLGLTGSQGAAICFGEKLILVGETPDEKAVQYLLQWLENREVQDVFFTSSLSQIYPDAVNFKSVASGLLAIPIARHNFLLWFRPEVLQTVNWGGDPNHAYEATQEDGKIELHPRQSFDLWKEIVRLQSLPWQSVEIQSALALKKAIVNLILRQAEETGGSGVDYPYDVPDYALD

SEQ ID NO: 67 - protein, 921 aa, Artificial
  redOpto-rtrkB
MGSSKSKPKDPSQRLDVTGKLARHSKFGMKGPASVISNDDDSASPLHHISNGSNTPSSSEGGPDAVIIGMTKIPVIENPQYFGITNSQLKPDTFVQHIKRHNIVLKRELGEGAFGKVFLAECYNLCPEQDKILVAVKTLKDASDNARKDFHREAELLTNLQHEHIVKFYGVCVEGDPLIMVFEYMKHGDLNKFLRAHGPDAVLMAEGNPPTELTQSQMLHIAQQIAAGMVYLASQHFVHRDLATRNCLVGENLLVKIGDFGMSRDVYSTDYYRVGGHTMLPIRWMPPESIMYRKFTTESDVWSLGVVLWEIFTYGKQPWYQLSNNEVIECITQGRVLQRPRTCPQEVYELMLGCWQREPHTRKNIKNIHTLLQNLAKASPVYLDILGTGGATTVQLSDQSLRQLETLAIHTAHLIQPHGLVVVLQEPDLTISQISANCTGILGRSPEDLLGRTLGEVFDSFQIDPIQSRLTAGQISSLNPSKLWARVMGDDFVIFDGVFHRNSDGLLVCELEPAYTSDNLPFLGFYHMANAALNRLRQQANLRDFYDVIVEEVRRMTGFDRVMLYRFDENNHGDVIAEDKRDDMEPYLGLHYPESDIPQPARRLFIHNPIRVIPDVYGVAVPLTPAVNPSTNRAVDLTESILRSAYHCHLTYLKNMGVGASLTISLIKDGHLWGLIACHHQTPKVIPFELRKACEFFGRVVFSNISAQEDTETFDYRVQLAEHEAVLLDKMTTAADFVEGLTNHPDRLLGLTGSQGAAICFGEKLILVGETPDEKAVQYLLQWLENREVQDVFFTSSLSQIYPDAVNFKSVASGLLAIPIARHNFLLWFRPEVLQTVNWGGDPNHAYEATQEDGKIELHPRQSFDLWKEIVRLQSLPWQSVEIQSALALKKAIVNLILRQAEEPGGSGVDYPYDVPDYALD

SEQ ID NO: 68 - DNA, 2928 bp, Artificial
  redOpto-mFGFR1
atggggagtagcaagagcaagcctaaggaccccagccagcgcctcgacatgaagagcggcaccaagaagagcgacttccatagccagatggctgtgcacaagctggccaagagcatccctctgcgcagacaggtaacagtgtcagctgactccagtgcatccatgaactctggggttctcctggttcggccctcacggctctcctccagcgggacccccatgctggctggagtctccgaatatgagctccctgaggatccccgctgggagctgccacgagacagactggtcttaggcaaaccacttggcgagggctgcttcgggcaggtggtgttggctgaggccatcgggctggataaggacaaacccaaccgtgtgaccaaagtggccgtgaagatgttgaagtccgacgcaacggagaaggacctgtcggatctgatctcggagatggagatgatgaaaatgattgggaagcacaagaatatcatcaaccttctgggagcgtgcacacaggatggtcctctttatgtcattgtggagtacgcctccaaaggcaatctccgggagtatctacaggcccggaggcctcctgggctggagtactgctataaccccagccacaaccccgaggaacagctgtcttccaaagatctggtatcctgtgcctatcaggtggctcggggcatggagtatcttgcctctaagaagtgtatacaccgagacctggctgctaggaacgtcctggtgaccgaggataacgtaatgaagatcgcagactttggcttagctcgagacattcatcatatcgactactacaagaaaaccaccaacggccggctgcctgtgaagtggatggcccctgaggcgttgtttgaccggatctacacacaccagagcgatgtgtggtcttttggagtgctcttgtgggagatcttcactctgggtggctccccataccccggtgtgcctgtggaggaacttttcaagctgctgaaggagggtcatcgaatggacaagcccagtaactgtaccaatgagctgtacatgatgatgcgggactgctggcatgcagtgccctctcagagacctacgttcaagcagttggtggaagacctggaccgcattgtggccttgacctccaaccaggagtatctggacctgtccataccgctggaccagtactcacccagctttcccgacacacggagctccacctgctcctcaggggaggactctgtcttctctcatgagccgttacctgaggagccctgtctgcctcgacaccccacccagcttgccaacagtggactcaaacggcgcgtcgagaccggggcaactactgttcaactgtctgatcaatctctgcgtcaactggaaactctggctatccacaccgcgcatctgatccagccgcacggtctggtagtcgtcctgcaagaaccggacctgaccatcagccagatctctgcgaactgtaccggtatcctgggccgtagcccggaagatctgctgggtcgtactctgggcgaggtattcgattcttttcagattgatccgatccagtctcgtctgaccgcaggtcagatttccagcctgaacccgtccaagctgtgggcgcgtgttatgggtgacgactttgttattttcgacggcgtatttcatcgtaactctgatggcctgctggtttgcgagctggagccggcctacactagcgacaacctgcctttcctgggtttctaccatatggcaaacgcggcactgaaccgtctgcgtcagcaagctaacctgcgcgacttctacgacgttatcgttgaggaagtgcgccgcatgacgggtttcgaccgcgtcatgctgtaccgttttgatgaaaacaaccacggtgacgtaatcgcggaggataagcgtgacgacatggagccgtatctgggtctgcactacccggaaagcgacattcctcagccggcacgtcgcctgttcattcacaacccgatccgtgttattccggacgtttacggcgttgctgttccgctgactccggccgttaatccgtctactaaccgtgcagttgacctgaccgaatccatcctgcgttccgcataccattgccacctgacctatctgaagaacatgggcgttggtgctagcctgacgatctctctgattaaagatggtcacctgtggggtctgatcgcttgccatcaccagaccccgaaagtaatccctttcgaactgcgtaaagcctgcgaattcttcggtcgtgtggtgttctctaatatctccgcgcaagaagacaccgagacttttgactaccgcgtacagctggcggagcatgaagcggttctgctggacaaaatgaccaccgcggcagacttcgtggagggcctgactaaccacccagaccgtctgctgggcctgaccggcagccaaggcgctgcgatttgtttcggcgagaaactgattctggtgggcgaaaccccagacgaaaaggcggtgcaatacctgctgcaatggctggagaatcgcgaagtgcaggacgttttcttcactagctctctgtctcagatctatccggatgcggttaacttcaaaagcgtggcgtccggcctgctggctatcccgatcgcccgtcataactttctgctgtggttccgcccggaggttctgcagaccgttaattggggtggtgatccgaatcacgcatacgaagcaacccaagaagatggtaagatcgaactgcatccgcgtcagtccttcgatctgtggaaagaaattgttcgcctgcagagcctgccgtggcagagcgttgagatccagtctgccctggctctgaagaaagcaatcgtgaacctgattctgcgccaagctgaagaacccggtggatccggagtcgactatccgtacgacgtaccagactacgcactcgactaa

SEQ ID NO: 69 - DNA, 2766 bp, Artificial
  redOpto-rtrkB
atggggagtagcaagagcaagcctaaggaccccagccagcgcctcgacgtcaccggaaagttggcgagacattccaagtttggcatgaaaggcccagcttccgtcatcagcaacgacgatgactctgccagccctctccaccacatctccaacgggagcaacactccgtcttcttcggagggcgggcccgatgctgtcatcattgggatgaccaagatccctgtcattgaaaacccccagtacttcggtatcaccaacagccagctcaagccggacacatttgttcagcacatcaagagacacaacatcgttctgaagagggagcttggagaaggagcctttgggaaagttttcctagcggagtgctataacctctgccccgagcaggataagatcctggtggccgtgaagacgctgaaggacgccagcgacaatgctcgcaaggactttcatcgcgaagccgagctgctgaccaacctccagcacgagcacattgtcaagttctacggtgtctgtgtggagggcgacccactcatcatggtctttgagtacatgaagcacggggacctcaacaagttccttagggcacacgggccagatgcagtgctgatggcagagggtaacccgcccaccgagctgacgcagtcgcagatgctgcacatcgctcagcaaatcgcagcaggcatggtctacctggcatcccaacacttcgtgcaccgagacctggccacccggaactgcttggtaggagagaacctgctggtgaaaattggggacttcgggatgtcccgggatgtatacagcaccgactactaccgggttggtggccacacaatgttgcccatccgatggatgcctccagagagcatcatgtacaggaaattcaccaccgagagtgacgtctggagcctgggagttgtgttgtgggagatcttcacctacggcaagcagccctggtatcagctatcaaacaacgaggtgatagaatgcatcacccagggcagagtccttcagcggcctcgcacgtgtccccaggaggtgtacgagctgatgctgggatgctggcagcgggaaccacacacaaggaagaacatcaagaacatccacacactccttcagaacttggcgaaggcgtcgcccgtctacctggacatcctaggcaccggtggagcaactactgttcaactgtctgatcaatctctgcgtcaactggaaactctggctatccacaccgcgcatctgatccagccgcacggtctggtagtcgtcctgcaagaaccggacctgaccatcagccagatctctgcgaactgtaccggtatcctgggccgtagcccggaagatctgctgggtcgtactctgggcgaggtattcgattcttttcagattgatccgatccagtctcgtctgaccgcaggtcagatttccagcctgaacccgtccaagctgtgggcgcgtgttatgggtgacgactttgttattttcgacggcgtatttcatcgtaactctgatggcctgctggtttgcgagctggagccggcctacactagcgacaacctgcctttcctgggtttctaccatatggcaaacgcggcactgaaccgtctgcgtcagcaagctaacctgcgcgacttctacgacgttatcgttgaggaagtgcgccgcatgacgggtttcgaccgcgtcatgctgtaccgttttgatgaaaacaaccacggtgacgtaatcgcggaggataagcgtgacgacatggagccgtatctgggtctgcactacccggaaagcgacattcctcagccggcacgtcgcctgttcattcacaacccgatccgtgttattccggacgtttacggcgttgctgttccgctgactccggccgttaatccgtctactaaccgtgcagttgacctgaccgaatccatcctgcgttccgcataccattgccacctgacctatctgaagaacatgggcgttggtgctagcctgacgatctctctgattaaagatggtcacctgtggggtctgatcgcttgccatcaccagaccccgaaagtaatccctttcgaactgcgtaaagcctgcgaattcttcggtcgtgtggtgttctctaatatctccgcgcaagaagacaccgagacttttgactaccgcgtacagctggcggagcatgaagcggttctgctggacaaaatgaccaccgcggcagacttcgtggagggcctgactaaccacccagaccgtctgctgggcctgaccggcagccaaggcgctgcgatttgtttcggcgagaaactgattctggtgggcgaaaccccagacgaaaaggcggtgcaatacctgctgcaatggctggagaatcgcgaagtgcaggacgttttcttcactagctctctgtctcagatctatccggatgcggttaacttcaaaagcgtggcgtccggcctgctggctatcccgatcgcccgtcataactttctgctgtggttccgcccggaggttctgcagaccgttaattggggtggtgatccgaatcacgcatacgaagcaacccaagaagatggtaagatcgaactgcatccgcgtcagtccttcgatctgtggaaagaaattgttcgcctgcagagcctgccgtggcagagcgttgagatccagtctgccctggctctgaagaaagcaatcgtgaacctgattctgcgccaagctgaagaacccggtggatccggagtcgactatccgtacgacgtaccagactacgcactcgactaa

SEQ ID NO: 70 - DNA, 36 bp, Artificial
  redOpto-mFGFR1_inverse_F
gatccaccggtggagcaactactgttcaactgtctg

SEQ ID NO: 71 - DNA, 26 bp, Artificial
  rtrkB_ICD_BspEI_F
gatctccggaaagtttggcatgaaag

SEQ ID NO: 72 - DNA, 26 bp, Artificial
  rtrkB_ICD_AgeI_R
cagaaaccggtgcctaggatgtccag

(19) United States
(12) Patent Application Publication
(10) Pub. No.: US 2016/0326219 A1
(43) Pub. Date: Nov. 10, 2016

(54) OPTICALLY ACTIVATED RECEPTORS

(71) Applicants: IST AUSTRIA (INSTITUTE OF SCIENCE AND TECHNOLOGY AUSTRIA), Klosterneuburg (AT); MEDIZINISCHE UNIVERSITAET WIEN, Wien (AT)

(72) Inventors: Robert RIEDLER, Vienna (AT); Eva REICHHART, Vienna (AT); Christopher DIFFER, Kirkintilloch (GB); Alvaro Ingles PRIETO, Vienna (AT); Harald JANOVJAK, Vienna (AT); Michael GRUSCH, Vienna (AT); Karin SCHELCH, Vienna (AT)

(73) Assignees: IST AUSTRIA (INSTITUTE OF SCIENCE AND TECHNOLOGY AUSTRIA), Klosterneuburg (AT); MEDIZINISCHE UNIVERSITAET WIEN, Wien (AT)

(21) Appl. No.: 15/103,588

(22) PCT Filed: Dec. 12, 2014
(86) PCT No.: PCT/EP2014/077556

§ 371 Date: Jun. 10, 2016

(30) Foreign Application Priority Data
Dec. 13, 2013 (EP) 13197174.9
May 14, 2014 (EP) 14168286.4

(51) Int. Cl. C07K 14/195; C12N 5/071; C12N 5/00; C07K 14/71; G01N 33/50

(52) U.S. Cl. CPC C07K 14/195; C07K 14/71; G01N 33/5041; C12Y 207/10001; G01N 33/5088; C12N 5/00; C12N 5/0602; C07K 2319/00; C07K 2319/60; G01N 2333/912; G01N 2333/195; C12N 2501/10; C12N 2529/10; C12N 2506/00; C12N 2510/00

(57) ABSTRACT

The present invention belongs to the field of biotechnology. More specifically, the invention relates to chimeric fusion proteins comprising a light activated protein domain, e.g., a newly characterized light-oxygen-voltage-sensing (LOV) domain or a light sensing domain of the cyanobacterial phytochrome (PHY) CPH1, wherein the chimeric fusion protein is capable of dimerizing upon excitation with light of a suitable wavelength. Said fusion proteins further comprise the intracellular part of a receptor tyrosine kinase (RTK). The invention further relates to nucleic acid molecules encoding said chimeric fusion proteins; non-human transgenic animals expressing the chimeric fusion protein encoded by said nucleic acid molecules; as well as uses of said chimeric fusion proteins, e.g. in a screening method.

The present invention belongs to the field of biotechnology, in particular the field of optogenetics. More specifically, the invention relates to chimeric fusion proteins comprising a light activated protein domain, e.g., a newly characterized light-oxygen-voltage-sensing (LOV) domain or the light sensing domain of the cyanobacterial phytochrome (PHY) CPH1, wherein the chimeric fusion protein is capable of dimerizing upon excitation with light of a suitable wavelength. Said fusion proteins can further comprise the intracellular part of a cell surface receptor. The invention further relates to nucleic acid molecules encoding said chimeric fusion proteins; non-human transgenic animals expressing the chimeric fusion protein encoded by said nucleic acid molecules; as well as uses of said chimeric fusion proteins, e.g. in a screening method.

### BACKGROUND OF THE INVENTION

In the emerging field of optogenetics, light-activated proteins are exploited to modulate cells of higher organisms with spatial and temporal precision, precise control of intensity and no need to physically connect stimulus and responding element given that the matrix is sufficiently transparent. Inspired by naturally-occurring proteins or building on light-sensitive chemical entities, membrane currents, G-protein signaling, membrane recruitment, gene expression and protein function are now amenable to optical control in living cells (Fenno, Yizhar et al. 2011, Tucker 2012).

Cells respond to extracellular signaling molecules through the activation of cell surface receptors and intracellular multi-component signaling pathways. Receptor tyrosine kinases (RTKs) are a large family of transmembrane receptors that sense growth factors and hormones and are key regulators of normal and aberrant physiology (Lemmon and Schlessinger 2010). Activation of RTKs is tightly regulated and restricted to distinct subcellular locations, cell types and developmental stages while dysregulation is prominently linked to human disease (Robertson et al. 2000, Shilo 2005, Casaletto and McClatchey 2012). These spatial and temporal complexities of RTK signaling call for novel investigative approaches that offer precise control of receptor activation and downstream effects. In contrast to ion channels and G-protein coupled receptors, for some of which spatial and temporal control in cells and tissues is offered by light-activated proteins (Szobota and Isacoff 2010, Fenno et al. 2011), RTKs and their associated signaling pathways can currently not be optically controlled. Moreover, while it was proposed that the non-invasive nature of optical “remote” control may be taken advantage of in the evaluation of pharmacological compounds (Prigge et al. 2010, Entcheva 2013), this proposal has not been experimentally realized to date and may be most desirable in the context of disease-related signaling pathways.

In the first step of RTK activation, extracellular ligands induce or stabilize receptor homo- or heterodimers. Dimerization activates kinase domains by an allosteric interaction that results in trans-phosphorylation and that propagates the signal to intracellular multicomponent pathways (Lemmon and Schlessinger 2010, Simi and Ibanez 2010). Using bivalent antibodies, crosslinking mutations and chemical functionalization, it was shown that dimerization is sufficient for ligand-independent activation of several RTKs (Spaargaren et al. 1991, Burke and Stern 1998, Muthuswamy et al. 1999, Welm et al. 2002). Although receptor dimerization is a common molecular activation mechanism in RTKs and other receptor families (Cochran et al. 2001), methods for the selective and spatio-temporally precise control of receptor dimerization are currently not available.

Wend et al. (2013) disclose chimeric fusion proteins of the protein kinases C-RAF or B-RAF, which are involved in the MAP-kinase pathway, and the N-terminal region of the protein cryptochrome-interacting basic-helix-loop-helix 1 (CIBN) or the protein cryptochrome 2 (CRY2). The proteins dimerize upon excitation with blue light.

WO 2012/116621 discloses an optically controlled gene expression system.

WO 2009/151948 describes a combination of a first protein of interest fused to a phytochrome domain, and a second protein of interest fused to a phytochrome domain interacting peptide. Both proteins dimerize upon excitation with red light.

WO 2010/006049, WO 2008/089003, WO 2008/086470, WO 2007/024391, and US 2010/0234273 disclose light activated ion channels.

WO 2013/003557 and WO 2009/148946 disclose fusion proteins of the light-activated G-protein coupled receptor rhodopsin and G-protein coupled receptors of other families.

WO 1999/036553 discloses multimeric chimeric proteins which can be dimerized by a chemical ligand. US 2009/0233364 describes pathway effectors which can be dimerized using a leucine zipper.

GenBank entry NGA_0015702 (Uniprot sequence K8Z861) discloses the sequence of an uncharacterized hypothetical protein from Nannochloropsis gaditana. Uniprot sequence C5NSW6 discloses a putative aureochrome1-like protein from Ochromonas danica.

Huang et al. (2013) describe the cloning of full-length aureochrome 1 from Nanochloropsis gaditana and its expression in Saccharomyces cerevisiae.

Strauss et al. (2005) propose that dimerization of the cyanobacterial phytochrome (PHY) CPH1 of Synechocystis PCC6803 (SyCP1-PHY) plays an important role in effector domain regulation.

US 2013/0116165, and Möglich et al. Photochem. Photobiol. Sci. 9: 1286-1300 (2010) describe the light sensitive proteins Avena sativa LOV domain (AsLOV), AsLOV-cp and Vivid. Pathak et al. Biol. Cell 105: 59-72 (2013), and Müller & Weber, Mol. Biosyst. 9: 596-608 (2013) discuss LOV domains in general and in particular fusion proteins comprising an AsLOV-domain. Schmidt et al. Nature Communications, DOI: 10.1038/ncomms4019 (August 2013) describes fusions of AsLOV with a Kv channel-specific peptide toxin, which can be used to modulate cellular K⁺ current. AsLOV, AsLOV-cp and Vivid show 42% or less sequence identity to NgPA1-LOV, 43% or less sequence identity to OdPA1-LOV and VfAU1-LOV, and 13% or less sequence identity to SyCP1-PHY, respectively.

WO 2013/074911 discloses a light responsive DNA binding protein comprising a LOV domain derived from E. litoralis 222 (EL222-LOV), and a DNA binding domain in operative linkage to a transcriptional activation domain. EL222-LOV exhibits 34% or less sequence identity to NgPA1-LOV, OdPA1-LOV and VfAU1-LOV, and 10% sequence identity to SyCP1-PHY.

Stroh et al. Stem Cells 29: 78-88 (2011) describe automated optogenetic stimulation of embryonic stem cells by using the light-inducible ion channel channelrhodopsin-2. These transmembrane ion channel proteins are very remote from LOV domains, they do not dimerize upon excitation with light, and have a complete different mechanism of action.

WO 2013/133643 discloses fusion proteins of the C-terminus of receptor tyrosine kinases and light-sensitive proteins, such as CIB (cryptochrome-interacting basic-helix-loop-helix protein), CIBN (N-terminal domain of CIB), Phy (phytochrome), PIF (phytochrome interacting factor), FKF1 (Flavin-binding, Kelch repeat, F-box 1), GIGANTEA, CRY (chryptochrome), PHR (phytolyase homolgous region). Significant differences exist in the protein sequences despite similar names. The following table displays sequence identities (%) of the light-sensitive proteins.

| | NgPA1-LOV | OdPA1-LOV | VfAU1-LOV | SyCP1-PHY |
|---|---|---|---|---|
| CIB | 4 | 4 | 4 | 3 |
| CIBN | 3 | 5 | 4 | 4 |
| Phy | 9 | 14 | 19 | 26 |
| PIF | 4 | 7 | 3 | 6 |
| FKF1 | 30 | 34 | 31 | 5 |
| GIGANTEA | 15 | 16 | 15 | 4 |
| CRY | 15 | 5 | 7 | 8 |
| PHR | 15 | 5 | 3 | 6 |

Also differences in experimental function and performance exist. None of the light-sensitive domains mentioned in WO 2013/133643 can be used for homodimerization of RTKs. CRY and its shorter form PHR do not produce receptor-doublets, but receptor multiplets as they homooligomerize. Homooligomerization is ‘less controllable’ than homodimerization as homodimers have a defined composition (doublet) while oligomers may have many different possible compositions (multiplets). CIBN heterodimerizes with CRY2, PIF1-6 heterodimerizes with PHYA or PHYB, and FKF1 heterodimerizes with Gigantea. Heterodimers have the technical disadvantage that their expression requires two genes, which in most settings is very difficult to achieve. In addition, in a publication corresponding to WO 2013/133643 (Kim et al. Chem Biol. 21: 903-912 (2014)) it is stated in the section ‘Live Cell Imaging and Photoactivation’ that the required activation intensity for the mentioned light sensing domains was 1.30-64.94 mW/cm². Finally, WO 2013/133643 is completely silent on light sensing domains which can be activated by red light.

US 2006/0110827 describes the SyCP1-PHY domain and mutants thereof; it does not address light-induced dimerization but a different physical phenomenon (fluorescence).

There is a need in the art for tools capable of spatial and temporal control of signaling in a cell. In particular, there is a need for new optogenetic tools, which enable novel light controllable applications.

### SUMMARY OF THE INVENTION

The inventors reasoned that a light-activated protein-protein interaction engineered into RTKs may mimic ligand-induced dimerization and ultimately result in receptor activation. In one embodiment, the inventors selected blue light-sensing protein domains that belong to the large light-oxygen-voltage-sensing (LOV) domain superfamily as candidates for light-activated dimerization of RTKs. Light-sensing LOV domains bind flavins as prosthetic groups and act as reversible photoswitches in bacteria, fungi and plants. LOV-domain-containing photoreceptors control functionally heterogeneous effector domains such as serine/threonine kinases (e.g. in the flowering plant Arabidopsis thaliana (Kinoshita et al. 2001) or the green alga Chlamydomonas reinhardtii (Huang et al. 2002) or transcriptional regulators (e.g. in the fungus Neurospora crassa (Heintzen et al. 2001) or in the yellow-green alga Vaucheria frigida (Takahashi et al. 2007). Dimerization of LOV domains was proposed to play an important role in effector domain regulation and LOV domains exhibit remarkable diversity in their dimerization interfaces and interaction lifetimes (Zoltowski and Gardner 2011).

Moreover, the inventors extended this work with additional fusion proteins that are activated by red light (˜660 nm) and inactivated by far-red light (˜750 nm). In particular, the inventors identified a protein domain that undergoes homodimerization in response to red light (the light-sensing domain of the cyanobacterial phytochrome (PHY) CPH1 of Synechocystis PCC6803 (SyCP1-PHY)) and incorporated this domain into RTK fusion proteins. Red light penetrates animal tissue deeper than blue light; therefore, it can be applied externally. This novel tool is particularly attractive for optogenetic applications in animal models of development and diseases. The ability to remote control the activation and inactivation of specific proteins in vivo offers unprecedented insight into understanding biological processes.

The fusion proteins of the present invention are very light sensitive. Activation with NgPA1-LOV, OdPA1-LOV and VfAU1-LOV can already be achieved with blue light of 0.25 mW/cm², while activation with SyCP1-PHY is even already achieved with red light of 5.8 μW/cm²=0.0058 mW/cm².

The inventors engineered light-activated receptor tyrosine kinases that are genetically-encoded in their entirety and capable of spatial and temporal control of signaling in a cellular model of human disease, and the all-optical evaluation of pharmacological compounds in a disease-related signaling process was experimentally realized.

Accordingly, disclosed is a chimeric fusion protein, comprising a LOV domain having an amino acid sequence with at least 76% sequence identity to SEQ ID NO: 12 (NgPA1-LOV), wherein the chimeric fusion protein is capable of dimerizing, when the LOV domain is excited with light of a suitable wavelength. Further embodiments of said chimeric fusion protein are as described herein below, and as defined in the claims.

Further disclosed is a chimeric fusion protein, comprising a LOV domain having an amino acid sequence with at least 74% sequence identity to SEQ ID NO: 14 (OdPA1-LOV), wherein the chimeric fusion protein is capable of dimerizing, when the LOV domain is excited with light of a suitable wavelength. Further embodiments of said chimeric fusion protein are as described herein below, and as defined in the claims.

Also disclosed is a chimeric fusion protein, comprising a light sensing domain having an amino acid sequence with at least 70% sequence identity over the whole length to SEQ ID NO: 64 (SyCP1-PHY), in functional linkage with a chromophore, wherein the chimeric fusion protein is capable of dimerizing, when the light sensing domain is excited with light of a suitable wavelength.

The present disclosure also relates to a nucleic acid molecule encoding the chimeric fusion protein as described herein and as defined in the claims.

The present disclosure also pertains to a non-human transgenic animal, which expresses the chimeric fusion protein encoded by said nucleic acid molecule.

The present disclosure further relates to a screening method, comprising the steps of
a) providing a cell which expresses a chimeric fusion protein, comprising
   a LOV domain having an amino acid sequence with at least 70% sequence identity over the whole length of an amino acid sequence selected from SEQ ID NO: 12 (NgPA1-LOV), SEQ ID NO: 14 (OdPA1-LOV) and SEQ ID NO: 10 (VfAU1-LOV), and the intracellular part of a cell surface receptor,
   wherein the chimeric fusion protein is capable of dimerizing upon excitation of the LOV domain with light of a suitable wavelength, thereby triggering a cell response via said intracellular part of said cell surface receptor;
b) contacting said cell with a candidate agent;
c) exposing said cell with said light of a suitable wavelength; and
d) determining whether said candidate agent is capable of affecting said cell response triggered in step c).

Also described is a screening method, comprising the steps of
a) providing a cell which expresses a chimeric fusion protein, comprising
   a light sensing domain having an amino acid sequence with at least 70% sequence identity over the whole length to the amino acid sequence of SEQ ID NO: 64 (SyCP1-PHY), in functional linkage with a chromophore, and
   the intracellular part of a cell surface receptor,
   wherein the chimeric fusion protein is capable of dimerizing upon excitation of the light sensing domain with light of a suitable wavelength, thereby triggering a cell response via said intracellular part of said cell surface receptor;
b) contacting said cell with a candidate agent;
c) exposing said cell with said light of a suitable wavelength; and
d) determining whether said candidate agent is capable of affecting said cell response triggered in step c).

Further details and embodiments of said screening methods are provided below, and in the claims.

In addition, the present disclosure provides uses of the chimeric fusion protein as described herein. For example, the chimeric fusion protein as disclosed herein may be used as a research tool, preferably for characterizing an orphan receptor. Alternatively, the chimeric fusion protein as disclosed herein may be used in a screening method, preferably wherein the screening method uses light as an activator of said chimeric fusion protein and for the read-out of said screening method. The chimeric fusion protein as disclosed herein may also be used for producing patterned cell cultures, or it may be used for controlling the production of a biologic product of interest.

Further disclosed are non-therapeutic uses of the chimeric fusion protein as disclosed herein, e.g. for controlling cell growth or for controlling growth factor pathways, preferably wherein said chimeric fusion protein is used in vitro. Another non-therapeutic use of the chimeric fusion protein as disclosed herein is in the differentiation of stem cells, wherein the stem cell is not produced using a process which involves modifying the germ line genetic identity of human beings or which involves use of a human embryo for industrial or commercial purposes, preferably wherein said chimeric fusion protein is used in vitro.

The above uses and non-therapeutic uses are not limited to the chimeric fusion proteins discloses herein as such. Therefore, the present disclosure also discloses to the use of the nucleic acid molecule as disclosed herein as a research tool, preferably for characterizing an orphan receptor. Likewise, the use of the nucleic acid molecule as disclosed herein in a screening method is disclosed, preferably wherein the screening method uses light as an activator of said chimeric fusion protein and for the read-out of said screening method.

Finally, the use of the non-human transgenic animal as described herein as a research tool, preferably for characterizing an orphan receptor, and the use of the non-human transgenic animal as described herein in a screening method, is also disclosed.

Further details and preferred embodiments are set out in the detailed description and claims as attached.

### DETAILED DESCRIPTION OF THE PREFERRED EMBODIMENTS

Light-Oxygen-Voltage-sensing (LOV) domains are sensors for environmental conditions used by a large variety of higher plants, microalgae, fungi and bacteria. As a common feature, all LOV proteins comprise a blue-light sensitive flavin mononucleotide chromophore, which is covalently linked to the protein core via an adjacent cysteine residue in the signaling state. LOV domains are e.g. encountered in blue-light-sensitive protein complexes regulating a great diversity of biological processes.

Disclosed is a chimeric fusion protein, comprising a LOV domain having an amino acid sequence with at least 76% sequence identity to SEQ ID NO: 12 (N. gaditana hypothetical protein NGA_0015702, residue 87 to 228 of Uniprot sequence K8Z861 (NgPA1-LOV)), wherein the chimeric fusion protein is capable of dimerizing, when the LOV domain is excited with light of a suitable wavelength.

Preferably, the LOV domain of said fusion protein has an amino acid sequence with at least 78%, more preferably 80%, more preferably at least 85%, more preferably at least 90%, more preferably at least 95%, more preferably at least 96%, more preferably at least 97%, more preferably at least 98%, more preferably at least 99%, and most preferably 100% sequence identity over the whole length of the amino acid sequence of SEQ ID NO: 12 (NgPA1-LOV).

Further disclosed is a chimeric fusion protein, comprising a LOV domain having an amino acid sequence with at least 74% sequence identity to SEQ ID NO: 14 (O. danica aureochrome1 like protein, residue 180 to 312 of Uniprot sequence C5NSW6 (OdPA1-LOV)), wherein the chimeric fusion protein is capable of dimerizing, when the LOV domain is excited with light of a suitable wavelength.

Preferably, the LOV domain of said further fusion protein has an amino acid sequence with at least 75%, preferably at least 76%, more preferably 78%, more preferably 80%, more preferably at least 85%, more preferably at least 90%, more preferably at least 95%, more preferably at least 96%, more preferably at least 97%, more preferably at least 98%, more preferably at least 99%, and most preferably 100% sequence identity over the whole length of the amino acid sequence of SEQ ID NO: 14 (OdPA1-LOV).

Also disclosed is a chimeric fusion protein, comprising a light sensing PHY domain having an amino acid sequence with at least 70% sequence identity over the whole length to SEQ ID NO: 64 (SyCP1-PHY), in functional linkage with a chromophore, wherein the chimeric fusion protein is capable of dimerizing, when the light sensing domain is excited with light of a suitable wavelength. Preferably, the light sensing domain has an amino acid sequence with at least 78%, more preferably 80%, more preferably at least 85%, more preferably at least 90%, more preferably at least 95%, more preferably at least 96%, more preferably at least 97%, more preferably at least 98%, more preferably at least 99%, and most preferably 100% sequence identity over the whole length to the amino acid sequence of SEQ ID NO: 64 (SyCP1-PHY).

As used herein, an amino acid sequence is said to have “X % sequence identity with SEQ ID NO: Y” over the whole length of the sequence, if the sequence in question is aligned with said SEQ ID NO: Y and the sequence identity between those to aligned sequences is at least X % over the whole length of SEQ ID NO: Y. Alignments of amino acid sequences can be performed using publicly available computer homology programs such as the “BLAST” program, “blastp”, provided at the NCBI homepage at http://www.ncbi.nlm.nih.gov/blast/blast.cgi, using the default settings provided therein. Identical residues are determined, e.g., by counting by hand, and a subsequent calculation of the percentage identity (PID) by dividing the number of identities over the indicated length of SEQ ID NO: Y gives “X % sequence identity”. If a particular length is not specifically indicated, the sequence identity is calculated over the entire/full length of SEQ ID NO: Y. Alternative methods of calculating sequence identity percentages of sets of polypeptides are known in the art. In a preferred embodiment, the changes in the amino acid sequence, e.g. substitution(s), insertion(s) or deletion(s), which result in at least X % identity to SEQ ID NO: Y are of a minor nature. More specifically, the amino acid sequence differing from SEQ ID NO: Y preferably comprises one or more semi-conservative and more preferably conservative amino acid substitutions, or combinations thereof. Semi-conservative and conservative substitutions of a given amino acid residue are provided in the below table.

| Amino acid | Conservative exchange | Semi-conservative exchange |
|---|---|---|
| A | G; S; T | N; V; C |
| C | A; V; L | M; I; F; G |
| D | E; N; Q | A; S; T; K; R; H |
| E | D; Q; N | A; S; T; K; R; H |
| F | W; Y; L; M; H | I; V; A |
| G | A | S; N; T; D; E; N; Q; |
| H | Y; F; K; R | L; M; A |
| I | V; L; M; A | F; Y; W; G |
| K | R; H | D; E; N; Q; S; T; A |
| L | M; I; V; A | F; Y; W; H; C |
| M | L; I; V; A | F; Y; W; C; |
| N | Q | D; E; S; T; A; G; K; R |
| P | V; I | L; A; M; W; Y; S; T; C; F |
| Q | N | D; E; A; S; T; L; M; K; R |
| R | K; H | N; Q; S; T; D; E; A |
| S | A; T; G; N | D; E; R; K |
| T | A; S; G; N; V | D; E; R; K; I |
| V | A; L; I | M; T; C; N |
| W | F; Y; H | L; M; I; V; C |
| Y | F; W; H | L; M; I; V; C |

Substituting A, F, H, I, L, M, P, V, W or Y by C is semi-conservative if the new cysteine remains as a free thiol. Substituting M to either E, R or K is considered semi-conservative, if the ionic tip of the new side group can reach the protein surface while the methylene groups make hydrophobic contacts. Substituting P by one of K, R, E or D is semi-conservative, if the side group is located on the protein surface. Moreover, it will be understood that glycines at sterically demanding positions may not be substituted and that P should not be introduced into alpha-helical or a beta sheet structures. Residues critical for the structure and activity of the LOV domain, and which may therefore not be made subject of substitutions, can be identified by methods well-known in the art, e.g. alanine-scanning mutagenesis.

The term “chimeric fusion protein” as used herein is intended to mean that the fusion protein is a genetically engineered fusion protein, which otherwise would not exist in nature. The fusion protein may be derived from, and thus composed of, at least two different parent proteins. However, the fusion protein may also be derived from more than two parent proteins, such as three, four, five or even six different parent proteins. The parent proteins may be native to each other, but preferably said parent proteins are foreign to each other, i.e. they naturally occur in different species. Fusion proteins are made by fusing the coding region of one parent protein (or a part encoding a domain or truncated version of said parent protein) in frame with the coding region of another parent protein (or a part encoding a domain or truncated version of said other parent protein). However, it is also contemplated that the fusion protein comprises two or more domains of the same protein, fused in a manner that would not exist in nature.

The chromophore of the PHY domain is a linear tetrapyrrole, four pyrroles linked together in a linear molecule with then varying substituents. Preferably, the linear tetrapyrrole is a linear tetrapyrrole occurring in nature, e.g. a linear tetrapyrrole selected from phycocyanonbilin, phycoerythrobilin, phycourobilin, phycoviolobilin, phytochromobilin, biliverdin, bilirubin, mesobiliverdin, mesobilirubin, bilane, bilin, urobilin, stercobilin, and urobilinogen. Most preferably the chromophore is phycocyanonbilin.

Upon excitation with light of a suitable wavelength, the LOV domains, and thus the fusion protein, will dimerize. LOV domains are usually excitable by blue light, i.e. by light having a wavelength in the range of 350-500 nm, preferably in the range of 400-500 nm, more preferably in the range of about 420 nm to about 490 nm. However, the present disclosure may also encompass LOV domains, which have been mutated in order to shift the wavelength of the light necessary for excitation of said domain. However, the skilled person will either know the wavelength suitable for excitation of the LOV domain, or will be readily capable of determining which light to be used for excitation of the LOV domain by routine methods.

The LOV domains of the chimeric fusion proteins disclosed herein are quite light sensitive. In a preferred embodiment, the LOV domain is capable of being activated at 5 μW/mm² of light, preferably 4 μW/mm² of light, more preferably 3 μW/mm² of light, and most preferably 2.5 μW/mm² of light. It could be further demonstrated that the LOV domains disclosed herein are also capable of being activated at 2.0 μW/mm², 1.5 μW/mm², 1.0 μW/mm², 0.5 μW/mm², and 0.3 μW/mm² of light.

Likewise, upon excitation with light of a suitable wavelength the light sensing PHY domain, and thus the fusion protein will dimerize, preferably homodimerize. In contrast to LOV domains, PHY domains are excitable by red light, i.e. by light having a wavelength in the range of 600-690 nm, preferably 610-680 nm, more preferably in the range of 620-670 nm, and most preferably in the range of 630-660 nm, such as by light having a wavelength of about 650 nm. In addition, the light sensing PHY domain can be inactivated by light with a wavelength in the range of 700-750 nm, preferably 710-740 nm, more preferably 720-730 nm. The light sensing PHY domain is even more light sensitive than the LOV domain. To that end, the light sensing domain is capable of being activated at 0.5 μW/mm² of light, preferably 0.4 μW/mm² of light, more preferably 0.3 μW/mm² of light, and most preferably 0.25 μW/mm² of light, such as at 0.2 μW/mm², 0.15 μW/mm², 0.1 μW/mm², 0.05 μW/mm², and 0.03 μW/mm² of light.

Whether the fusion protein is capable of dimerization can be tested using any suitable assay known in the art. The choice of the assay will depend on the fusion partner of the LOV domain or PHY domain. In case of a known effector protein which elicits its effector function upon dimerization, such as a tyrosine kinase receptor, capability for dimerization may be tested by determining the activation of downstream signaling molecules. This may be accomplished using methods known in the art such as determining the functional state of the signaling molecules, e.g., by using antibodies directed against phosphotyrosine. Alternatively, one may also determine a specific final effect of the elicited signaling as such, i.e. a cell response such as a change in cell cycle distribution, a change in the transcriptional profile of the cell, localization and distribution of specific proteins in the cell, a change in the phenotype of the cells such as in the shape of the cells, a change in the distribution of cells on a surface or in three dimensional structures, a change in metabolic activity of the cells; by determining percentage survival or death of the cells, by determining the differentiation state of the cells and/or by determining a change in the composition of metabolites of the cells. Assays for determining such effector functions will be describe further below. Alternatively, one may also design a reporter gene construct, which reporter gene becomes expressed upon dimerization of the chimeric fusion protein. If the fusion partner is not characterized yet, one may either determine phenotypic changes of a cell expressing the chimeric fusion protein described herein as compared to a mock-transfected control cell, or one may use techniques for determining dimerization which are independent of the fusion partner, such as fluorescence resonance energy transfer (FRET), or any other suitable method known in the art. FRET is a mechanism describing energy transfer between two fluorescence chromophores. A donor chromophore, may transfer in its excited state energy to an acceptor chromophore. The efficiency of this energy transfer is inversely proportional to the distance between donor and acceptor making FRET extremely sensitive to small distances. Thus, the skilled person will readily recognize suitable methods for determining whether the chimeric fusion protein is capable of dimerizing. The expression “capable of dimerization upon excitation” is, however, also intended to indicate that constitutive dimerization without previous excitation is excluded.

In a preferred embodiment, the chimeric fusion protein further comprises the intracellular part of a receptor tyrosine kinase (RTK). RTKs are the high-affinity cell surface receptors for many polypeptide growth factors, cytokines, and hormones. RTKs have been shown to be key regulators of normal cellular processes as well as to have a critical role in the development and progression of many types of cancer. Each RTK monomer has a single hydrophobic transmembrane domain composed of 25-38 amino acid residues, an intracellular C-terminal region, and an extracellular N-terminal region. In an even more preferred embodiment, the chimeric fusion protein may further comprise the transmembrane domain of said RTK, which allows the fusion protein to be incorporated into the cell membrane. The skilled person can readily determine the transmembrane domain from the amino acid sequence of the RTK.

The extracellular N-terminal region contains primarily a ligand-binding site, which binds extracellular ligands, such as a hormone or growth factor. The intracellular C-terminal region displays the highest level of conservation and comprises catalytic domains responsible for the signaling activity. Extracellular ligand binding will typically cause or stabilize receptor dimerization and lead to receptor autophosphorylation and/or tyrosine phosphorylation of its specific substrates, e.g. members of the MAP kinase signaling pathway.

With regard to LOV domain comprising chimeric fusion protein, the tyrosine kinase is preferably a RTK selected from the group consisting of EGF receptors (such as EGFR/ErbB1, ErbB2, ErbB3 or ErbB4), FGF receptors, RET receptors, insulin receptors, PDGF receptors, VEGF receptors, HGF receptors, Trk receptors, Eph receptors, AXL receptors, LTK receptors, TIE receptors, ROR receptors, DDR receptors, KLG receptors, RYK receptors, and MuSK receptors, more preferably from EGF receptors, FGF receptors and RET receptors, and most preferably from EGFR, FGFR1 and RET.

If the fusion protein comprises a PHY domain, the tyrosine kinase is preferably a RTK selected from the group consisting of FGF receptors, Trk receptors, EGF receptors (such as EGFR/ErbB1, ErbB2, ErbB3 or ErbB4), RET receptors, insulin receptors, PDGF receptors, VEGF receptors, HGF receptors, Eph receptors, AXL receptors, LTK receptors, TIE receptors, ROR receptors, DDR receptors, KLG receptors, RYK receptors, and MuSK receptors, more preferably from FGF receptors, Trk receptors, even more preferably from FGFR1 and TrkB.

In a most preferred embodiment, the fusion protein is redOpto-mFGFR1 (SEQ ID NO: 66) or redOpto-rtrkB (SEQ ID NO: 67) in particular as further described below. redOpto-mFGFR1 and redOpto-rtrkB exemplify a highly valuable class of optogenetic tools, since red light offers markedly improved tissue penetration compared to blue light. For instance, bone/skull of 5 mm thickness transmits ˜2% of blue (460 nm) but ˜10% of red (640 nm) light (Wan, Parrish et al. 1981). Or, muscle tissue of 1 cm thickness transmits ˜20% of blue but ˜80% of red light (Marquez, Wang et al. 1998). Thus, red light controlled RTKs enable non-invasive activation of the MAPK signaling pathway in cells.

Notably, the combination of PHY- and LOV domain-containing receptor families enables experiments with dual-color activation. Hence, chimeric fusion proteins comprising different light sensing domains (LOV and PHY domains) may be combined in all uses and methods disclosed herein.

The sequences, the intracellular parts, and the transmembrane domains of these RTKs are published in public sequence databases and well known in the art or can be easily determined using routine methods in the art. The chimeric fusion protein disclosed herein thus allows triggering of any receptor which becomes activated upon dimerization independent of its ligand. As a consequence, the chimeric fusion proteins disclosed herein are very valuable research tools, which e.g. allow the characterization of so called orphan receptors. Therefore, in another preferred embodiment, the chimeric fusion protein comprises the intracellular part of an orphan receptor.

Also disclosed is a chimeric fusion as disclosed herein, wherein the chimeric fusion protein is a transcription factor, further comprising a DNA-binding domain and a transcription regulating domain, which transcription factor in dimerized form is capable of promoting or repressing the transcription of a target gene comprising in functional linkage the recognition sequence of said DNA-binding domain. The DNA-binding domain recognizes and attaches to specific sequences of DNA adjacent to the genes that they regulate. Depending on the transcription regulating domain, the transcription factor may be an activator or repressor of the transcription of the gene. Transcription factors use a variety of mechanisms for the regulation of gene expression. These mechanisms include blocking or stabilizing the binding of RNA polymerase to DNA, acetylation or deacetylation of histones or by recruiting co-activator or co-repressor proteins to the promoter or enhancer region.

In a preferred embodiment, the LOV domain or PHY domain is positioned C-terminally from its fusion partner(s). In another preferred embodiment, the LOV domain or PHY domain is located at the C-terminus or the N-terminus of the fusion protein, more preferably the LOV domain or PHY domain is located at the C-terminus of the fusion protein.

The chimeric fusion protein may further comprise a fluorescence protein, which allows determining whether the chimeric fusion protein is expressed in a cell, as well as its localization in said cell. Preferred fluorescence proteins for use herein are GFP, EGFP, mCherry, or mVenus. However, any fluorescence protein suitable for the chimeric fusion proteins disclosed herein may be used. In the context of FRET, fluorescence proteins may be used for determining whether the chimeric fusion protein is capable of dimerization upon excitation with light of a suitable wavelength, as described elsewhere herein.

In a preferred embodiment, the chimeric fusion protein homodimerizes, when the LOV domain or PHY domain is excited with light of a suitable wavelength. However, it is also contemplated to provide two non-identical fusion proteins as disclosed herein, which heterodimerize via their (preferably identical) LOV domains or PHY domains upon excitation with light of a suitable wavelength.

Further disclosed is a nucleic acid molecule encoding the chimeric fusion protein as described herein and as defined in the claims.

The term “nucleic acid molecule” as used herein is known in the art and may refer to DNA, RNA, cDNA or hybrids thereof or any modification thereof. Nucleic acid residues comprised by the nucleic acid molecules described herein may be naturally occurring nucleic acid residues or artificially produced nucleic acid residues, such as adenine (A), guanine (G), cytosine (C), thymine (T), uracil (U), xanthine (X), and hypoxanthine (HX). Thymine (T) and uracil (U) may be used interchangeably depending on the respective type of polynucleotide, since thymine (T) in DNA corresponds to uracil (U) in transcribed mRNA. The nucleic acid molecule provided and described herein may be single- or double-stranded, linear or circular, natural or synthetic, and without any size limitation. The nucleic acid molecule may further comprise in functional linkage transcription regulating sequences, such as a promoter, and transcriptional and translational start and stop signals. In one particular embodiment, the nucleic acid molecule may be in the form of a vector. The term “vector” as used herein particularly refers to plasmids, cosmids, viruses, bacteriophages, transposons and other vectors commonly used in genetic engineering. In a preferred embodiment, the vector is suitable for the transformation of a cell, like microbiological cells, such as fungal cells, yeast cells or prokaryotic cells. The vector may be suitable for stable transformation of eukaryotic cells, in order to express the chimeric fusion protein disclosed herein. More preferably, the vector disclosed is an expression vector as generally known in the art. Preferably, the nucleic acid molecule or vector comprises a selectable marker, which allows selection for cells transformed with the nucleic acid molecule or vector. The nucleic acid molecule or vector may also comprise integrational elements, which allow integration of the nucleic acid molecule or vector into the genome of a host cell, e.g. by using homologous recombination. In addition or alternatively, the nucleic acid molecule or vector may also comprise an origin of replication, which allows the nucleic acid molecule to be maintained in a cell without the need of being integrated into the host cell's genome. Other means advantageous or necessary for use in combination with a nucleic acid molecule, as well as methods for ligating same, are generally known in the art. In one preferred embodiment, the nucleic acid molecule comprises, more preferably consists of the nucleic acid sequence of SEQ ID NO: 54. In another preferred embodiment, the nucleic acid molecule comprises, more preferably consists of the nucleic acid sequence of SEQ ID NO: 55. In still another preferred embodiment, the nucleic acid molecule comprises the nucleic acid sequence of SEQ ID NO: 65 (SyCP1-PHY), more preferably the nucleic acid molecule comprises the nucleic acid sequence of SEQ ID NO: 68 (redOpto-mFGFR1) or SEQ ID NO: 69 (redOpto-rtrkB). In a most preferred embodiment, the nucleic acid molecule consists of the nucleic acid sequence of SEQ ID NO: 68 (redOpto-mFGFR1) or SEQ ID NO: 69 (redOpto-rtrkB).

In this context, the present disclosure further provides a cell, such as an isolated cell, or a cell within an isolated tissue, which expresses the chimeric fusion protein as disclosed herein and/or which comprises the nucleic acid molecule as described herein. The host cell may be a prokaryotic or eukaryotic cell, comprising the nucleic acid molecule or the vector or a cell derived from such a cell and containing the nucleic acid molecule or the vector as disclosed herein. In a preferred embodiment, the host cell comprises, i.e. is genetically modified with, the nucleic acid molecule or the vector in such a way that it contains the nucleic acid molecule integrated into the genome. The host cell may be a bacterial, yeast, a fungus or a eukaryotic cell such as a mammalian cell or an insect cell. Transformation or genetically engineering of the host cell with a nucleic acid molecule or vector as disclosed herein can be carried out by standard methods known in the art.

Moreover, a non-human transgenic animal is disclosed, which expresses the chimeric fusion protein as disclosed herein and/or encoded by the nucleic acid molecule as described herein. The “transgenic non-human animal” may be any animal other than a human. In a preferred embodiment, the transgenic non-human animal is a vertebrate, preferably a mammal, more preferably a rodent, such as a mouse or a rat; or a non-human primate, i.e. a primate that is not a member of the genus Homo, for example rhesus macaque, chimpanzee, baboon, marmoset, and green monkey. The term “non-human transgenic animal” includes well-known model organisms, comprising, but not limited to guinea pig (Cavia porcellus), hamster, mouse (Mus musculus), and rat (Rattus norvegicus), Sigmodon hispidus, dog (Canis lupus familiaris), cat (Felis cattus), chicken (Gallus gallus domesticus), zebra finch (Taeniopygia guttata), african clawed frog (Xenopus laevis), Japanese ricefish (Oryzias latipes), pufferfish (Takifugu rubripres), Lamprey, zebrafish (Danio rerio), Caenorhabditis elegans, Arbacia punctulata, Ciona intestinalis, Drosophila, e.g. Drosophila melanogaster, Euprymna scolopes, Hydra, Loligo pealei, Pristionchus pacificus, Strongylocentrotus purpuratus, Symsagittifera roscoffensis, and Tribolium castaneum. The transgenic non-human animal can be heterozygous for the nucleic acid molecule, but in a preferred embodiment, the transgenic non-human animal is homozygous for the nucleic acid molecule. It is noted that those animals are excluded, which are not likely to yield in substantial benefit to man or animal and which are therefore not subject to patentability under the respective patent law or jurisdiction. The skilled person will take appropriate measures, as e.g. laid down in international guidelines of animal welfare, to ensure that the substantial benefit to man or animal will outweigh any animal suffering.

The chimeric fusion proteins as described herein are particularly useful in a screening method. For example, they allow the characterization of new receptors, abolish the need for expensive ligands, and allow a spatial and temporal control of receptor signalling.

Therefore, the present disclosure also provides a screening method, comprising the steps of
a) providing a cell which expresses a chimeric fusion protein, comprising
   a LOV domain having an amino acid sequence with at least 70% sequence identity over the whole length of an amino acid sequence selected from SEQ ID NO: 12 (NgPA1-LOV), SEQ ID NO: 14 (OdPA1-LOV) and SEQ ID NO: 10 (VfAU1-LOV), and the intracellular part of a cell surface receptor,
   wherein the chimeric fusion protein is capable of dimerizing upon excitation of the LOV domain with light of a suitable wavelength, thereby triggering a cell response via said intracellular part of said cell surface receptor;
b) contacting said cell with a candidate agent;
c) exposing said cell with said light of a suitable wavelength; and
d) determining whether said candidate agent is capable of affecting said cell response triggered in step c).

Likewise, provided is a screening method, comprising the steps of
a) providing a cell which expresses a chimeric fusion protein, comprising
   a light sensing domain having an amino acid sequence with at least 70% sequence identity over the whole length to the amino acid sequence of SEQ ID NO: 64 (SyCP1-PHY), in functional linkage with a chromophore, and
   the intracellular part of a cell surface receptor,
   wherein the chimeric fusion protein is capable of dimerizing upon excitation of the light sensing domain with light of a suitable wavelength, thereby triggering a cell response via said intracellular part of said cell surface receptor;
b) contacting said cell with a candidate agent;
c) exposing said cell with said light of a suitable wavelength; and
d) determining whether said candidate agent is capable of affecting said cell response triggered in step c).

The chimeric fusion protein of the above screening method may be further defined as described previously. Hence, in a preferred embodiment, the chimeric fusion protein comprises a LOV domain having an amino acid sequence with at least 70% sequence identity over the whole length to the amino acid sequence of SEQ ID NO: 12 (NgPA1-LOV). In a more preferred embodiment, the LOV domain has an amino acid sequence with at least 73%, preferably at least 75%, more preferably 80%, more preferably at least 85%, more preferably at least 90%, more preferably at least 95%, more preferably at least 96%, more preferably at least 97%, more preferably at least 98%, more preferably at least 99%, and most preferably 100% sequence identity over the whole length of the amino acid sequence of SEQ ID NO: 12 (NgPA1-LOV).

In another preferred embodiment, the chimeric fusion protein comprises a LOV domain having an amino acid sequence with at least 70% sequence identity over the whole length to the amino acid sequence of SEQ ID NO: 14 (OdPA1-LOV).

In a more preferred embodiment, the LOV domain has an amino acid sequence with at least 73%, preferably at least 75%, more preferably 80%, more preferably at least 85%, more preferably at least 90%, more preferably at least 95%, more preferably at least 96%, more preferably at least 97%, more preferably at least 98%, more preferably at least 99%, and most preferably 100% sequence identity over the whole length of the amino acid sequence of SEQ ID NO: 14 (OdPA1-LOV).

In still another preferred embodiment, the chimeric fusion protein comprises a LOV domain having an amino acid sequence with at least 70% sequence identity over the whole length to the amino acid sequence of SEQ ID NO: 10 (VfAU1-LOV). In a more preferred embodiment, the LOV domain has an amino acid sequence with at least 73%, preferably at least 75%, more preferably 80%, more preferably at least 85%, more preferably at least 90%, more preferably at least 95%, more preferably at least 96%, more preferably at least 97%, more preferably at least 98%, more preferably at least 99%, and most preferably 100% sequence identity over the whole length of the amino acid sequence of SEQ ID NO: 10 (VfAU1-LOV).

In another preferred embodiment, the light sensing domain has an amino acid sequence with at least 73%, preferably at least 75%, more preferably 80%, more preferably at least 85%, more preferably at least 90%, more preferably at least 95%, more preferably at least 96%, more preferably at least 97%, more preferably at least 98%, more preferably at least 99%, and most preferably 100% sequence identity over the whole length to the amino acid sequence of SEQ ID NO: 64 (SyCP1-PHY), and/or the chromophore is a linear tetrapyrrole, preferably selected from phycocyanonbilin, phycoerythrobilin, phycourobilin, phycoviolobilin, phytochromobilin, biliverdin, bilirubin, mesobiliverdin, mesobilirubin, bilane, bilin, urobilin, stercobilin, and urobilinogen, preferably wherein the chromophore is phycocyanonbilin.

In a preferred embodiment, the chimeric fusion protein homodimerizes, when the LOV domain is excited with light of a suitable wavelength. However, it is also contemplated to provide two non-identical fusion proteins as disclosed herein, which heterodimerize via their (preferably identical) LOV domains upon excitation with light of a suitable wavelength.

Preferably, the LOV domain is excitable by blue light, i.e. by light having a wavelength in the range of 350-500 nm, preferably in the range of 400-500 nm, more preferably in the range of about 420 nm to about 490 nm. However, the present disclosure may also encompass LOV domains, which have been mutated in order to shift the wavelength of the light necessary for excitation of said domain. Furthermore, the LOV domain is preferably capable of being activated at 5 μW/mm² of light, preferably 4 μW/mm² of light, more preferably 3 μW/mm² of light, and most preferably 2.5 μW/mm² of light. It could be further demonstrated that the LOV domains disclosed herein are also capable of being activated at 2.0 μW/mm², 1.5 μW/mm², 1.0 μW/mm², 0.5 μW/mm², and 0.3 μW/mm² of light.

As noted above, the PHY domains are excitable by red light, i.e. by light having a wavelength in the range of 600-690 nm, preferably 610-680 nm, more preferably in the range of 620-670 nm, and most preferably in the range of 630-660 nm, such as by light having a wavelength of about 650 nm. In addition, the light sensing PHY domain can be inactivated by light with a wavelength in the range of 700-750 nm, preferably 710-740 nm, more preferably 720-730 nm. The light sensing PHY domain is capable of being activated at 0.5 μW/mm² of light, preferably 0.4 μW/mm² of light, more preferably 0.3 μW/mm² of light, and most preferably 0.25 μW/mm² of light, such as at 0.2 μW/mm², 0.15 μW/mm², 0.1 μW/mm², 0.05 μW/mm², and 0.03 μW/mm² of light.

In a preferred embodiment, the LOV domain or PHY domain is positioned C-terminally from its fusion partner(s). In another preferred embodiment, the LOV domain or PHY domain is located at the C-terminus or the N-terminus of the fusion protein. In a most preferred embodiment, the LOV domain or PHY domain is located at the C-terminus of the chimeric fusion protein.

In a preferred embodiment of the screening method, said intracellular part of a receptor is the intracellular part of a receptor tyrosine kinase (RTK), as further described above. Said fusion protein may preferably further comprise the transmembrane domain of said RTK. As disclosed above with regard to LOV domains, examples of suitable RTKs are EGF receptors (such as EGFR/ErbB1, ErbB2, ErbB3 or ErbB4), FGF receptors, insulin receptors, PDGF receptors, VEGF receptors, HGF receptors, Trk receptors, Eph receptors, AXL receptors, LTK receptors, TIE receptors, ROR receptors, DDR receptors, RET receptors, KLG receptors, RYK receptors, and MuSK receptors. In a preferred embodiment, the chimeric fusion protein comprises the intracellular part of an EGF receptor, an FGF receptor or an RET receptor. In a more preferred embodiment, the chimeric fusion protein comprises the intracellular part of EGFR, FGFR1 or RET. In a most preferred embodiment, the chimeric fusion protein comprises SEQ ID NO: 58 (mFGFR1-VfAU1-LOV), SEQ ID NO: 59 (p75-hEGFR-VfAU1-LOV), or SEQ ID NO: 60 (hRET-VfAU1-LOV). In still a most preferred embodiment, the chimeric fusion protein consists of SEQ ID NO: 58 (mFGFR1-VfAU1-LOV), SEQ ID NO: 59 (p75-hEGFR-VfAU1-LOV), or SEQ ID NO: 60 (hRET-VfAU1-LOV).

If the fusion protein comprises a PHY domain, the tyrosine kinase is preferably a RTK selected from the group consisting of FGF receptors, Trk receptors, EGF receptors (such as EGFR/ErbB1, ErbB2, ErbB3 or ErbB4), RET receptors, insulin receptors, PDGF receptors, VEGF receptors, HGF receptors, Eph receptors, AXL receptors, LTK receptors, TIE receptors, ROR receptors, DDR receptors, KLG receptors, RYK receptors, and MuSK receptors, more preferably from FGF receptors, Trk receptors, even more preferably from FGFR1 and TrkB. In a most preferred embodiment, the fusion protein has at least 70%, more preferably at least 73%, preferably at least 75%, more preferably 80%, more preferably at least 85%, more preferably at least 90%, more preferably at least 95%, more preferably at least 96%, more preferably at least 97%, more preferably at least 98%, more preferably at least 99%, and most preferably 100% sequence identity over the whole length of the amino acid sequence of redOpto-mFGFR1 (SEQ ID NO: 66). In another most preferred embodiment, the fusion protein has at least 70%, more preferably at least 73%, preferably at least 75%, more preferably 80%, more preferably at least 85%, more preferably at least 90%, more preferably at least 95%, more preferably at least 96%, more preferably at least 97%, more preferably at least 98%, more preferably at least 99%, and most preferably 100% sequence identity over the whole length of the amino acid sequence of redOpto-rtrkB (SEQ ID NO: 67). In still a most preferred embodiment, the fusion protein consists of redOpto-mFGFR1 (SEQ ID NO: 66). In still another most preferred embodiment, the fusion protein consists of redOpto-rtrkB (SEQ ID NO: 67).

Alternatively, the chimeric fusion protein may also further comprise the intracellular part of an orphan receptor, as disclosed in further detail above.

Non limiting examples of “candidate agents” are small molecules, peptides, polypeptides, peptidomimetics, antibody molecules, as well as saccharide-, lipid-, and nucleic acid-based compounds. Small molecules may be derived from natural sources or may have been developed synthetically, e.g., by combinatorial chemistry. However, it will be understood that the precise source of the candidate agents is not decisive. Generally, the small molecule will have a molecular weight in the range of 250-800 Da, more preferably in the range of 300 to 750 Da, such as 350 to 700 Da, or 400 to 650 Da. Synthetic compound libraries and libraries of natural compounds in the form of bacterial, fungal, plant, and animal extracts are commercially available. Alternatively, such libraries may be generated, according to methods well known in the art.

Step d) is the step of determining whether said candidate agent is capable of affecting the cell response triggered in step c). Generally, any appropriate method or technique may be used in step d). More specifically, the choice of the method or technique will depend on the cell response triggered in step c). However, it is particularly preferred that step d) uses light as the read-out of the change in the cell response, i.e. that the cell response can be determined using optical sensors, which can be suitably applied in determining the strength and distribution of fluorescence signals.

For example, step d) may comprise determination of the gene transcriptional profile of the cell. Methods for determining the gene transcriptional profile of cells are known in the art. In a preferred embodiment, the gene is switched on or off in response to the cell response. In a more preferred embodiment, the gene transcription profile may be determined using a reporter gene, which is under the control of a regulatory sequence, which initiates transcription upon dimerization of the chimeric fusion protein. Examples of such reporter assays are described in the materials and methods section below and comprise the commercial Cignal Reporter Assay and the Path Detect Elk1 trans Reporting System, which use a luciferase reporter gene. Another example are reporter gene assays using other enzymes such as a galactosidases or esterases. Alternatively, one may subject the cell to an RNA- or cDNA microarray assay, semi-quantitative PCR, quantitative PCR or realtime PCR using primer and probes which are specific and/or characteristic for the cell response. To that end, the materials and methods section also describes a Western Blot, which may be applied likewise.

Step d) may also comprise determination of the cell cycle distribution. Methods for determining cell cycle distribution are known in the art. A method for determining cell cycle distribution is described in more detail in the methods and material section under the heading “Cell cycle distribution”. Additional methods for determining cell cycle distribution include analysis of the expression profile, as described below.

Step d) may also comprise determination of the localization of proteins in the cell. For example, one may measure the internalization of activated receptor proteins, or localization of characteristic nucleic proteins, e.g. transcription factors. Methods for determining localization of proteins in the cell are known in the art. This can be done by (i) fusing these proteins to fluorescent proteins or (ii) by labelling these proteins with fluorescent markers. Instead of fluorescence, particles made of gold or other metals can be used for detection. Fusion proteins or labelled proteins are localized with a wide range of microscopy techniques, e.g. fluorescence microscopy or electron microscopy. In addition, fluorescent proteins and fluorescent molecules can be used that respond to changes in pH with changes in optical properties and thereby allow for detection of the cellular compartment that the protein is in. Labelling can be achieved through reactions with antibodies, enzymes (e.g. “SNAP-tag”) or chemical-reactive groups.

In still another embodiment, step d) may comprise determination of the functional state of proteins in the cell, such as by determining the phosphorylation state of signaling molecules characteristic for the cell response triggered in step c). Methods for determining functional state of proteins are known in the art. Determining the functional state of proteins can be accomplished by extraction of the specific or all proteins from cells followed by labeling of protein specifically in one but not the other functional state. Labelling can be achieved using antibodies specific for functional protein states. In a different method, functional state can be identified by analyzing protein localization, as described above. In a different method, proteins can be fused to one or more fluorescent proteins, e.g. in FRET part, that respond to a change in functional state with a change in optical properties. In a different method, association of the protein with other proteins can be detected and used as a measure for functional state.

Likewise, step d) may comprise determination of the shape of cells. Methods for determining cell shape are known in the art. This can be accomplished by light or fluorescence microscopy. For the latter, cells may be stained appropriately, or they may either express a fluorescence protein or they may be labeled with a suitable fluorescent molecule or protein. An assay for determining cell morphology is further described in the materials and methods section below under the heading “Cell morphology”.

Alternatively, step d) may also be carried out by determining the distribution or the migratory behaviour of cells on a 2D surface or in 3D structure. Methods for determining distribution or the migratory behaviour of cells are known in the art. Distribution or migratory behaviour can be determined using light or fluorescence microscopy. Cells may be placed on a 2D surface or in a 3D structure. The position of each cell will be recorded, also as a function of time. From the position of each cell, parameters describing cell distribution (e.g. distance to nearest neighbour, number of neighbours within a certain area) or migratory behaviour (e.g. velocity of cell motion or distance traveled during a certain time) can be extracted.

Another embodiment of step d) comprises determination of the metabolic activity of the cells, or determination of the composition of metabolites of the cells. Methods for determining metabolic activity of cells are known in the art. Metabolic activity may, for example, be determined in terms of cell proliferation, as described in the materials and methods section below under the heading “Cell proliferation”. Metabolic activity may also be determined by analysing of cellular chemical composition, e.g. using mass spectrometry or methods of chromatography. Metabolic activity may also be determined using chemical agents that are processed by cells and for which processing depends on metabolic activity (e.g. tetrazolium dyes).

In still another embodiment, step d) comprises determination of the survival or death of the cells. Methods for determining survival or death of cells are known in the art, and may involve detection of a pro-apoptotic marker (e.g. annexin V or of caspases), incorporation of a dye into apoptotic cells (e.g. of propidium iodide), or determination of utilization of a substrate (e.g. [³H]-thymidin incorporation). Kits for determining percentage viable cells and/or apoptotic cells in a cell culture or sample are commercially available.

Step d) may alternatively comprise determination of the differentiation state of cells. Methods for determining the differentiation state of cells are known in the art. Determination of the differentiation state of the cells may involve determination of differentiation state specific cell markers, e.g. by flow cytometry, fluorescence microscopy or immunohistochemistry. Dependent on the type of differentiation, the cells may also undergo a change in cell morphology and/or in the expression profile, as described above.

In still another embodiment, step d) may comprise determining the incorporation of a nucleotide analogue by the cell. Methods for determining the incorporation of a nucleotide analogue by the cell are known in the art. The nucleotide analogue may be any suitable nucleotide analogue, which is capable of monitoring a cell response. For example, the nucleotide analogue may be 5-ethynyl-2′-deoxyuridine or bromodeoxyuridine. Detection of these analogues may be achieved using commercially available antibodies, or by fluorescence labelling, e.g. by labelling with a fluorescent molecule that features azide groups.

As demonstrated above, the chimeric fusion proteins disclosed herein can be advantageously incorporated into various applications. For example, the chimeric fusion proteins disclosed herein can be used as a research tool, preferably for characterizing an orphan receptor.

As described above, the chimeric fusion protein as disclosed herein can be used in a screening method. As a consequence, a screening method is provided, which may use light as an activator of said chimeric fusion protein and for the read-out of said screening method. This abolishes the need for adding a costly ligand, and thus allows advantageously applying the screening method in automated high-throughput screenings.

Moreover, the chimeric fusion protein a disclosed herein can be used in non-therapeutic applications for controlling cell growth, preferably wherein said chimeric fusion protein is used in vitro. For example, the chimeric fusion protein as disclosed herein can be used non-therapeutically for controlling growth factor pathways, preferably wherein said chimeric fusion protein is used in vitro.

Another application of the chimeric fusion protein as disclosed herein lies in the production of patterned cell cultures—or even patterned cell tissues. Patterned cell cultures are characterized in that certain cells of said cell cultures are stimulated, whereas others are not. The production of patterned cell cultures requires a high spatial control of activation, which is usually difficult to achieve when using the same culture medium for all cells. Due to its controllable excitation by light, one can use the chimeric fusion protein as disclosed herein for producing patterned cell cultures, as also demonstrated in the examples herein.

Besides the high spatial control, the chimeric fusion proteins disclosed herein also allow a high temporal control. High temporal control of receptor signalling pathways is for example required in the differentiation of stem cells, in which specific growth factor signalling pathways have to be applied at particular time points of differentiation to the cell. Hence, the chimeric fusion protein disclosed herein can be advantageously used in a non-therapeutic manner in the differentiation of stem cells, preferably wherein said chimeric fusion protein is used in vitro. Such stem cells can be obtained without using a process which involves modifying the germ line genetic identity of human beings or which involves use of a human embryo for industrial or commercial purposes.

The high spatial and temporal control of receptor activation makes the chimeric fusion protein as disclosed herein particularly useful in controlling the production of a biologic product of interest.

The above (non-therapeutic) uses are not limited to the chimeric fusion proteins disclosed herein as such. Similarly, the use of the nucleic acid molecule as disclosed herein, or of the non-human transgenic animal as disclosed herein, as a research tool is contemplated, preferably for characterizing an orphan receptor. Moreover, the nucleic acid molecule as disclosed herein, can be used in a screening method, preferably wherein the screening method uses light as an activator of said chimeric fusion protein encoded by the nucleic acid molecule and for the read-out of said screening method. Finally, it is also contemplated that the non-human transgenic animal as described herein is used in a screening method.

The method is further described by the following embodiments:
1. A screening method, comprising the steps of
   a) providing a cell which expresses a chimeric fusion protein, comprising
      a LOV domain having an amino acid sequence with at least 70% sequence identity over the whole length of an amino acid sequence selected from SEQ ID NO: 12 (NgPA1-LOV), SEQ ID NO: 14 (OdPA1-LOV) and SEQ ID NO: 10 (VfAU1-LOV), and
      the intracellular part of a cell surface receptor,
      wherein the chimeric fusion protein is capable of dimerizing upon excitation of the LOV domain with light of a suitable wavelength, thereby triggering a cell response via said intracellular part of said cell surface receptor;
   b) contacting said cell with a candidate agent;
   c) exposing said cell with said light of a suitable wavelength; and
   d) determining whether said candidate agent is capable of affecting said cell response triggered in step c).
2. The method of embodiment 1, wherein the LOV domain has an amino acid sequence with at least 73%, preferably at least 75%, more preferably 80%, more preferably at least 85%, more preferably at least 90%, more preferably at least 95%, more preferably at least 96%, more preferably at least 97%, more preferably at least 98%, more preferably at least 99%, and most preferably 100% sequence identity over the whole length of the amino acid sequence of SEQ ID NO: 12 (NgPA1-LOV).
3. The method of embodiment 1 or 2, wherein the LOV domain has an amino acid sequence with at least 73%, preferably at least 75%, more preferably 80%, more preferably at least 85%, more preferably at least 90%, more preferably at least 95%, more preferably at least 96%, more preferably at least 97%, more preferably at least 98%, more preferably at least 99%, and most preferably 100% sequence identity over the whole length of the amino acid sequence of SEQ ID NO: 14 (OdPA1-LOV).
4. The method of any one of embodiments 1-3, wherein the LOV domain has an amino acid sequence with at least 73%, preferably at least 75%, more preferably 80%, more preferably at least 85%, more preferably at least 90%, more preferably at least 95%, more preferably at least 96%, more preferably at least 97%, more preferably at least 98%, more preferably at least 99%, and most preferably 100% sequence identity over the whole length of the amino acid sequence of SEQ ID NO: 10 (VfAU1-LOV).
5. The method of any one of embodiments 1 to 4, wherein the chimeric fusion protein homodimerizes, when the LOV domain is excited with said light of a suitable wavelength.
6. The method of any one of embodiments 1 to 5, wherein the light for activating the LOV domain has a wavelength in the range of 350-500 nm.
7. The method of any one of embodiments 1 to 6, wherein the LOV domain is located at the C-terminus of the chimeric fusion protein.
8. The method of any one of embodiments 1 to 7, wherein the LOV domain is capable of being activated at 5 μW/mm² of light, preferably 4 μW/mm² of light, more preferably 3 μW/mm² of light, and most preferably 2.5 μW/mm² of light, such as at 2.0 μW/mm², 1.5 μW/mm², 1.0 μW/mm², 0.5 μW/mm², and 0.3 μW/mm² of light.
9. The method of any one of embodiments 1 to 8, wherein said intracellular part of a receptor is the intracellular part of a receptor tyrosine kinase (RTK).
10. The method of embodiment 9, wherein said fusion protein further comprises the transmembrane domain of said RTK.
11. The method of embodiment 9 or 10, wherein the tyrosine kinase is a RTK selected from the group consisting of EGF receptors (such as EGFR/ErbB1, ErbB2, ErbB3 or ErbB4), FGF receptors, RET receptors, insulin receptors, PDGF receptors, VEGF receptors, HGF receptors, Trk receptors, Eph receptors, AXL receptors, LTK receptors, TIE receptors, ROR receptors, DDR receptors, KLG receptors, RYK receptors, and MuSK receptors, preferably from EGF receptors, FGF receptors and RET receptors, more preferably from EGFR, FGFR1 and RET, and most preferably the fusion protein is selected from SEQ ID NO: 58 (mFGFR1-VfAU1-LOV), SEQ ID NO: 59 (p75-hEGFR-VfAU1-LOV), and SEQ ID NO: 60 (hRET-VfAU1-LOV).
12. The method of any one of embodiments 1 to 10, wherein the chimeric fusion protein further comprises the intracellular part of an orphan receptor.
13. The method of any one of embodiments 1 to 12, wherein step d) uses light as the read-out of the change in the cell response.
14. The method of any one of embodiments 1 to 13, wherein step d) comprises
   (i) determination of the cell cycle distribution, and/or
   (ii) determination of the gene transcriptional profile of the cell, and/or
   (iii) determination of the localization of proteins in the cell, and/or
   (iv) determination of the functional state of proteins in the cell, and/or
   (v) determination of the shape of cells, and/or
   (vi) determination of the distribution of cells on a surface or in 3D structure, and/or
   (vii) determination of the migratory behavior of cells on a surface or in 3D structure, and/or
   (viii) determination of the metabolic activity of cells, and/or
   (ix) determination of the survival or death of cells, and/or
   (x) determination of the differentiation state of cells, and/or
   (xi) determination of the composition of metabolites of cells, and/or
   (xii) determining the incorporation of a nucleotide analogue by the cell, preferably wherein the nucleotide analogue is 5-ethynyl-2′-deoxyuridine or bromodeoxyuridine, more preferably wherein the nucleotide analogue is fluorescent labelled or wherein the nucleotide analogues are detected by an antibody, most preferable wherein the fluorescent molecule are fluorescent azides.
15. The method of any one of embodiments 1 to 14, wherein step d) comprises determining the incorporation of a fluorescent nucleotide analogue by the cell, preferably wherein the fluorescent nucleotide analogue is 5-ethynyl-2′-deoxyuridine.
16. A chimeric fusion protein, comprising a LOV domain having an amino acid sequence with at least 76% sequence identity to SEQ ID NO: 12 (NgPA1-LOV), wherein the chimeric fusion protein is capable of dimerizing, when the LOV domain is excited with light of a suitable wavelength.
17. The chimeric fusion protein of embodiment 16, wherein the LOV domain has an amino acid sequence with at least 78%, more preferably 80%, more preferably at least 85%, more preferably at least 90%, more preferably at least 95%, more preferably at least 96%, more preferably at least 97%, more preferably at least 98%, more preferably at least 99%, and most preferably 100% sequence identity over the whole length of the amino acid sequence of SEQ ID NO: 12 (NgPA1-LOV).
18. A chimeric fusion protein, comprising a LOV domain having an amino acid sequence with at least 74% sequence identity to SEQ ID NO: 14 (OdPA1-LOV), wherein the chimeric fusion protein is capable of dimerizing, when the LOV domain is excited with light of a suitable wavelength.
19. The chimeric fusion protein of embodiment 18, wherein the LOV domain has an amino acid sequence with at least 75%, preferably at least 76%, more preferably 78%, more preferably 80%, more preferably at least 85%, more preferably at least 90%, more preferably at least 95%, more preferably at least 96%, more preferably at least 97%, more preferably at least 98%, more preferably at least 99%, and most preferably 100% sequence identity over the whole length of the amino acid sequence of SEQ ID NO: 14 (OdPA1-LOV).
20. The chimeric fusion protein of any one of embodiments 16-19, wherein the chimeric fusion protein homodimerizes, when the LOV domain is excited with light of a suitable wavelength.
21. The chimeric fusion protein of any one of embodiments 16 to 20, wherein the LOV domain is capable of being activated at 5 μW/mm² of light, preferably 4 μW/mm² of light, more preferably 3 μW/mm² of light, and most preferably 2.5 μW/mm² of light, such as at 2.0 μW/mm², 1.5 μW/mm², 1.0 μW/mm², 0.5 μW/mm², and 0.3 μW/mm² of light.
22. The chimeric fusion protein of any one of embodiments 16 to 21, wherein the light for activating the LOV domain has a wavelength in the range of 350-500 nm.
23. The chimeric fusion protein of any one of embodiments 16 to 22, wherein the LOV domain is located at the C-terminus of the chimeric fusion protein.
24. The chimeric fusion protein of any one of embodiments 16 to 23, wherein the chimeric fusion protein further comprises the intracellular part of a receptor tyrosine kinase (RTK).
25. The chimeric fusion protein of embodiment 24, wherein said fusion protein further comprises the transmembrane domain of said RTK.
26. The chimeric protein of embodiment 23 or 24, wherein the tyrosine kinase is a RTK selected from the group consisting of EGF receptors (such as EGFR/ErbB1, ErbB2, ErbB3 or ErbB4), FGF receptors, RET receptors, insulin receptors, PDGF receptors, VEGF receptors, HGF receptors, Trk receptors, Eph receptors, AXL receptors, LTK receptors, TIE receptors, ROR receptors, DDR receptors, KLG receptors, RYK receptors, and MuSK receptors, more preferably from EGF receptors, FGF receptors and RET receptors, and most preferably from EGFR, FGFR1 and RET.
27. The chimeric fusion protein of any one of embodiments 16 to 25, wherein the chimeric fusion protein further comprises the intracellular part of an orphan receptor.
28. The chimeric fusion protein of any one of embodiments 16 to 23, wherein the chimeric fusion protein is a transcription factor, further comprising a DNA-binding domain and a transcription regulating domain, which transcription factor in dimerized form is capable of promoting or repressing the transcription of a target gene comprising in functional linkage the recognition sequence of said DNA-binding domain.
29. The chimeric fusion protein of any one of embodiments 16 to 28, wherein the chimeric fusion protein comprises a fluorescence protein, preferably GFP, EGFP, mCherry, or mVenus.
30. A nucleic acid molecule encoding the chimeric fusion protein as defined in any one of embodiments 16 to 29.
31. The nucleic acid molecule of embodiment 30, comprising the nucleic acid sequence of SEQ ID NO: 54.
32. The nucleic acid molecule of embodiment 30, comprising the nucleic acid sequence of SEQ ID NO: 55.
33. A non-human transgenic animal, which expresses the chimeric fusion protein encoded by the nucleic acid molecule according to any one of embodiments 30-32.
34. Use of the chimeric fusion protein according to any one of embodiments 16 to 29 as a research tool, preferably for characterizing an orphan receptor.
35. Use of the chimeric fusion protein according to any one of embodiments 16 to 29 in a screening method, preferably wherein the screening method uses light as an activator of said chimeric fusion protein and for the read-out of said screening method.
36. Non therapeutic use of the chimeric fusion protein according to any one of embodiments 16 to 29 for controlling cell growth, preferably wherein said chimeric fusion protein is used in vitro.
37. Use of the chimeric fusion protein according to any one of embodiments 16 to 29 for producing patterned cell cultures.
38. Non-therapeutic use of the chimeric fusion protein according to any one of embodiments 16 to 29 for controlling growth factor pathways, preferably wherein said chimeric fusion protein is used in vitro.
39. Use of the chimeric fusion protein according to any one of embodiments 16 to 29 for controlling the production of a biologic product of interest.
40. Non-therapeutic use of the chimeric fusion protein according to any one of embodiments 16 to 29 in the differentiation of stem cells, wherein the stem cell is not produced using a process which involves modifying the germ line genetic identity of human beings or which involves use of a human embryo for industrial or commercial purposes, preferably wherein said chimeric fusion protein is used in vitro.
41. Use of the nucleic acid molecule according to any one of embodiments 30-32 as a research tool, preferably for characterizing an orphan receptor.
42. Use of the nucleic acid molecule according to embodiment 29 in a screening method, preferably wherein the screening method uses light as an activator of said chimeric fusion protein and for the read-out of said screening method.
43. Use of the non-human transgenic animal according to any one of embodiments 30-32 as a research tool, preferably for characterizing an orphan receptor.
44. Use of the non-human transgenic animal according to any one of embodiments 30-32 in a screening method.
45. A screening method, comprising the steps of
   a) providing a cell which expresses a chimeric fusion protein, comprising
      a light sensing domain having an amino acid sequence with at least 70% sequence identity over the whole length to the amino acid sequence of SEQ ID NO: 64 (SyCP1-PHY), in functional linkage with a chromophore, and
      the intracellular part of a cell surface receptor,
      wherein the chimeric fusion protein is capable of dimerizing upon excitation of the light sensing domain with light of a suitable wavelength, thereby triggering a cell response via said intracellular part of said cell surface receptor;
   b) contacting said cell with a candidate agent;
   c) exposing said cell with said light of a suitable wavelength; and
   d) determining whether said candidate agent is capable of affecting said cell response triggered in step c).
46. The method of embodiment 45, wherein the light sensing domain has an amino acid sequence with at least 73%, preferably at least 75%, more preferably 80%, more preferably at least 85%, more preferably at least 90%, more preferably at least 95%, more preferably at least 96%, more preferably at least 97%, more preferably at least 98%, more preferably at least 99%, and most preferably 100% sequence identity over the whole length to the amino acid sequence of SEQ ID NO: 64 (SyCP1-PHY).
47. The method of embodiment 45 or 46, wherein the chromophore is a linear tetrapyrrole, preferably selected from phycocyanonbilin, phycoerythrobilin, phycourobilin, phycoviolobilin, phytochromobilin, biliverdin, bilirubin, mesobiliverdin, mesobilirubin, bilane, bilin, urobilin, stercobilin, and urobilinogen, preferably wherein the chromophore is phycocyanonbilin.
48. The method of any one of embodiments 45 to 47, wherein the chimeric fusion protein homodimerizes when the light sensing domain is excited with said light of a suitable wavelength.
49. The method of any one of embodiments 45 to 48, wherein the light for activating the light sensing domain has a wavelength in the range of 600-690 nm.
50. The method of any one of embodiments 45 to 49, wherein the light for inactivating the light sensing domain has a wavelength in the range of 700-750 nm.
51. The method of any one of embodiments 45 to 50, wherein the light sensing domain is located at the C-terminus of the chimeric fusion protein.
52. The method of any one of embodiments 45 to 51, wherein the light sensing domain is capable of being activated at 0.5 μW/mm² of light, preferably 0.4 μW/mm² of light, more preferably 0.3 μW/mm² of light, and most preferably 0.25 μW/mm² of light, such as at 0.2 μW/mm², 0.15 μW/mm², 0.1 μW/mm², 0.05 μW/mm², and 0.03 μW/mm² of light.
53. The method of any one of embodiments 45 to 52, wherein said intracellular part of a receptor is the intracellular part of a receptor tyrosine kinase (RTK).
54. The method of embodiment 53, wherein said fusion protein further comprises the transmembrane domain of said RTK.
55. The method of embodiment 53 or 54 wherein the tyrosine kinase is a RTK selected from the group consisting of FGF receptors, Trk receptors, EGF receptors (such as EGFR/ErbB1, ErbB2, ErbB3 or ErbB4), RET receptors, insulin receptors, PDGF receptors, VEGF receptors, HGF receptors, Eph receptors, AXL receptors, LTK receptors, TIE receptors, ROR receptors, DDR receptors, KLG receptors, RYK receptors, and MuSK receptors, preferably from FGF receptors, Trk receptors, more preferably from FGFR1 and TrkB, and most preferably the fusion protein is redOpto-mFGFR1 (SEQ ID NO: 66) or redOpto-rtrkB (SEQ ID NO: 67).
56. The method of any one of embodiments 45 to 54, wherein the chimeric fusion protein further comprises the intracellular part of an orphan receptor.
57. The method of any one of embodiments 45 to 56, wherein step d) uses light as the read-out of the change in the cell response.
58. The method of any one of embodiments 45 to 57, wherein step d) comprises
   (i) determination of the cell cycle distribution, and/or
   (ii) determination of the gene transcriptional profile of the cell, and/or
   (iii) determination of the localization of proteins within the cell, and/or
   (iv) determination of the functional state of proteins in the cell, and/or
   (v) determination of the shape of cells, and/or
   (vi) determination of the distribution of cells on a surface or in 3D structure, and/or
   (vii) determination of the migratory behavior of cells on a surface or in 3D structure, and/or
   (viii) determination of the metabolic activity of cells, and/or
   (ix) determination of the survival or death of cells, and/or
   (x) determination of the differentiation state of cells, and/or
   (xi) determination of the composition of metabolites of cells, and/or
   (xii) determining the incorporation of a nucleotide analogue by the cell, preferably wherein the nucleotide analogue is 5-ethynyl-2′-deoxyuridine or bromodeoxyuridine, more preferably wherein the nucleotide analogue is fluorescent labelled or wherein the nucleotide analogues are detected by an antibody, most preferable wherein the fluorescent molecule are fluorescent azides.
59. The method of any one of embodiments 45 to 58, wherein step d) comprises determination of the gene transcriptional profile of the cell, more preferably using a reporter gene assay, most preferably using a luciferase reporter gene assay.
60. The method of any one of embodiments 45 to 58, wherein step d) comprises determining the incorporation of a fluorescent nucleotide analogue by the cell, preferably wherein the fluorescent nucleotide analogue is 5-ethynyl-2′-deoxyuridine.
61. A chimeric fusion protein, comprising a light sensing domain having an amino acid sequence with at least 70% sequence identity over the whole length to SEQ ID NO: 64 (SyCP1-PHY), in functional linkage with a chromophore, wherein the chimeric fusion protein is capable of dimerizing, when the light sensing domain is excited with light of a suitable wavelength.
62. The chimeric fusion protein of embodiment 61, wherein the light sensing domain has an amino acid sequence with at least 78%, more preferably 80%, more preferably at least 85%, more preferably at least 90%, more preferably at least 95%, more preferably at least 96%, more preferably at least 97%, more preferably at least 98%, more preferably at least 99%, and most preferably 100% sequence identity over the whole length to the amino acid sequence of SEQ ID NO: 64 (SyCP1-PHY).
63. The chimeric fusion protein of embodiment 61 or 62, wherein the chromophore is a linear tetrapyrrole, preferably selected from phycocyanonbilin, phycoerythrobilin, phycourobilin, phycoviolobilin, phytochromobilin, biliverdin, bilirubin, mesobiliverdin, mesobilirubin, bilane, bilin, urobilin, stercobilin, and urobilinogen, most preferably wherein the chromophore is phycocyanonbilin.
64. The chimeric fusion protein of any one of embodiments 61 to 63, wherein the chimeric fusion protein homodimerizes, when the light sensing domain is excited with light of a suitable wavelength.
65. The chimeric fusion protein of any one of embodiments 61 to 64, wherein the light sensing domain is capable of being activated at 0.5 μW/mm² of light, preferably 0.4 μW/mm² of light, more preferably 0.3 μW/mm² of light, and most preferably 0.25 μW/mm² of light, such as at 0.2 μW/mm², 0.15 μW/mm², 0.1 μW/mm², 0.05 μW/mm², and 0.03 μW/mm² of light.
66. The chimeric fusion protein of any one of embodiments 61 to 65, wherein the light for activating the light sensing domain has a wavelength in the range of 600-690 nm.
67. The chimeric fusion protein of any one of embodiments 61 to 66, wherein the light for inactivating the light sensing domain has a wavelength in the range of 700-750 nm
68. The chimeric fusion protein of any one of embodiments 61 to 67, wherein the light sensing domain is located at the C-terminus of the chimeric fusion protein.
69. The chimeric fusion protein of any one of embodiments 61 to 68, wherein the chimeric fusion protein further comprises the intracellular part of a receptor tyrosine kinase (RTK).
70. The chimeric fusion protein of embodiment 69, wherein said fusion protein further comprises the transmembrane domain of said RTK.
71. The chimeric protein of embodiment 69 or 70, wherein the tyrosine kinase is a RTK selected from the group consisting of FGF receptors, Trk receptors, EGF receptors (such as EGFR/ErbB1, ErbB2, ErbB3 or ErbB4), RET receptors, insulin receptors, PDGF receptors, VEGF receptors, HGF receptors, Eph receptors, AXL receptors, LTK receptors, TIE receptors, ROR receptors, DDR receptors, KLG receptors, RYK receptors, and MuSK receptors, more preferably from FGF receptors, Trk receptors, even more preferably from FGFR1 and TrkB, and most preferably the fusion protein is redOpto-mFGFR1 (SEQ ID NO: 66) or redOpto-rtrkB (SEQ ID NO: 67).
72. The chimeric fusion protein of any one of embodiments 61 to 70, wherein the chimeric fusion protein further comprises the intracellular part of an orphan receptor.
73. The chimeric fusion protein of any one of embodiments 61 to 68, wherein the chimeric fusion protein is a transcription factor, further comprising a DNA-binding domain and a transcription regulating domain, which transcription factor in dimerized form is capable of promoting or repressing the transcription of a target gene comprising in functional linkage the recognition sequence of said DNA-binding domain.
74. The chimeric fusion protein of any one of embodiments 61 to 73, wherein the chimeric fusion protein comprises a fluorescence protein, preferably GFP, EGFP, mCherry, or mVenus.
75. A nucleic acid molecule encoding the chimeric fusion protein as defined in any one of embodiments 61 to 74.
76. The nucleic acid molecule of embodiment 75, comprising the nucleic acid sequence of SEQ ID NO: 65 (SyCP1-PHY).
77. The nucleic acid molecule of embodiment 76, comprising the nucleic acid sequence of SEQ ID NO: 68 (redOpto-mFGFR1) or SEQ ID NO: 69 (redOpto-rtrkB).
78. A non-human transgenic animal, which expresses the chimeric fusion protein encoded by the nucleic acid molecule according to any one of embodiments 74-77.
79. Use of the chimeric fusion protein according to any one of embodiments 61 to 74 as a research tool, preferably for characterizing an orphan receptor.
80. Use of the chimeric fusion protein according to any one of embodiments 61 to 74 in a screening method, preferably wherein the screening method uses light as an activator of said chimeric fusion protein and for the read-out of said screening method.
81. Non-therapeutic use of the chimeric fusion protein according to any one of embodiments 61 to 74 for controlling cell growth, preferably wherein said chimeric fusion protein is used in vitro.
82. Use of the chimeric fusion protein according to any one of embodiments 61 to 74 for producing patterned cell cultures.
83. Non-therapeutic use of the chimeric fusion protein according to any one of embodiments 61 to 74 for controlling growth factor pathways, preferably wherein said chimeric fusion protein is used in vitro.
84. Non-therapeutic use of the chimeric fusion protein according to any one of embodiments 61 to 74 for controlling the production of a biologic product of interest.
85. Non-therapeutic use of the chimeric fusion protein according to any one of embodiments 61 to 74 in the differentiation of stem cells, wherein the stem cell is not produced using a process which involves modifying the germ line genetic identity of human beings or which involves use of a human embryo for industrial or commercial purposes, preferably wherein said chimeric fusion protein is used in vitro.
86. Use of the nucleic acid molecule according to any one of embodiments 75-77 as a research tool, preferably for characterizing an orphan receptor.
87. Use of the nucleic acid molecule according to embodiment 74 in a screening method, preferably wherein the screening method uses light as an activator of said chimeric fusion protein and for the read-out of said screening method.
88. Use of the non-human transgenic animal according to any one of embodiments 75-77 as a research tool, preferably for characterizing an orphan receptor.
89. Use of the non-human transgenic animal according to any one of embodiments 75-77 in a screening method.

In the following, the present invention is further illustrated by figures and examples, which are not intended to limit the scope of the present invention. All references cited herein are explicitly incorporated by reference.

### DESCRIPTION OF THE FIGURES

FIG. 1. Selection of LOV domains and expression in mammalian cells.

(a) Domain structure of light-sensing proteins from which LOV domains (highlighted with asterisk) were excised (AtPH1 and AtPH2: A. thaliana phototropin 1 and 2, CrPH: C. rheinhardtii phototropin, NcVV: N. crassa vivid, VfAU1: V. frigida aureochrome1). In these proteins, LOV domains regulate a variety of effector domains (STK: Ser/Thr kinase, DB: DNA-binding domain). To test for expression and influence on cell viability in mammalian cells, LOV domains were fused to the fluorescent protein mVenus (mV).

(b and d) Fluorescence intensity measurements of human embryonic kidney 293 (HEK293) cells (b) and chinese hamster ovary (CHO) K1 cells (d) transfected with mVenus-LOV domain fusions.

(c and e) Viability of HEK293 cells (c) and CHO K1 cells (e) transfected with mVenus-LOV domain fusions.

In b to e, data were normalized to mV fused to the small, robustly folding FK506 binding protein (FKBP).

FIG. 2. Design and function of mFGFR1-LOV domain fusion proteins in HEK293 cells.

(a) RTKs such as mFGFR1 consist of the extracellular ligand-binding domain (LBD), single-span transmembrane domain (TMD) and intracellular domain (ICD) (kinase domain (KD) and a C-terminal tail domain (CTD)). In mFGFR1-LOV domain fusion proteins, only the ICD is retained to render the protein insensitive to endogenous ligand. The ICD is attached to the membrane using a myristoylation domain (MYR) and LOV domains are incorporated at the ICD C-terminus.

(b) MAPK pathway activation in response to blue light for cells that express chimeric proteins of mFGFR1-ICD and LOV domains. imFGFR1 (see main text) is activated by the small molecule dimerizer AP20187.

(c) MAPK pathway activation in response to blue, green and red light for cells that express imFGFR1, Opto-mFGFR1 (mFGFR1-VfAU1-LOV) or kinase dead Opto-mFGFR1 (Y271F, Y272F).

FIG. 3. Pathway activation by Opto-mFGFR1 in HEK293 cells in response to blue light. Activation is expressed as induction of luciferase reporter gene (RLU of illuminated cells divided by RLU of cells kept in the dark). Light intensity was ˜3 μW/mm².

FIG. 4. Dimerization of Opto-mFGFR1 and VfAU1-LOV.

(a) Dimerization is required for Opto-mFGFR1 activation as introduction of the R195E mutation abolishes activation of MAPK pathway for imFGFR1 and Opto-mFGFR1. (b) VfAU1-LOV dimerizes in mammalian cells. Incorporation of VfAU1-LOV into a transcription factor that requires dimerization for activity (GA-VfAU1-LOV-P) yields light activated transcriptional responses. Gene design and positive control (pGAVPO) are described in the section headed “Materials and Methods”. Light intensity was ˜3 μW/mm².

FIG. 5. Fusion proteins of hEGFR, hRET and alternative LOV domains.

(a) Chimeric proteins of mFGFR1 and NgPA1-LOV or OdPA1-LOV respond to blue light with MAPK pathway activation.

(b) Chimeric proteins of hEGFR1-ICD or hRET-ICD and VfAU1-LOV respond to blue light with MAPK pathway activation.

(c) Reduced activation of Opto-mFGFR1 by blue light after reduction of VfAU1-LOV excited state lifetime. In a to c, light intensity was ˜3 μW/mm².

FIG. 6. Optical control of cancer cell behavior.

(a) Opto-mFGFR1 and ERK1/2 phosphorylation in human malignant pleural mesothelioma cells (M38K, SPC212) in response to blue light.

(b) AKT and PLCy1 phosphorylation in SPC212 cells in response to blue light.

(c) M38K cells respond to blue light with increased proliferation.

(d) M38K cells respond to blue light with increased percentage of cells in S-phase.

(e) M38K cells respond to blue light and FGF2 with elongated morphology.

(f) Representative images for (e).

(g) M38K cells respond to blue light with reduction of cortical actin and the emergence of long actin-rich filopodia in M38K cells.

(h) M38K cells respond to blue light with reduced expression of the epithelial marker E-cadherin and elevated expression of the mesenchymal marker vimentin and the EMT-associated transcription factors SNAIL1 and ZEB1. In a to h, light intensity was ˜3 μW/mm².

FIG. 7. Optical control of blood epithelial cell behavior.

(a) mFGFR1-VfAU1-LOV and ERK1/2 phosphorylation in response to blue light.

(b) hBE cell spheroids respond to blue light with sprouting. Control cells express mCherry.

(c) Representative images for (b). In a to c, light intensity was ˜3 μW/mm².

FIG. 8. Patterned illumination.

(a) Spatially-confined ERK1/2 phosphorylation in SPC212 cells. Scale bar is 2 mm.

(b) Spatially-confined ERK1/2 phosphorylation in hBE cells. Scale bar is 5 mm.

(c) Spatially-confined MAPK-dependent gene transcription in HEK293 cells. Scale bar is 10 mm. For a to c, all images are unprocessed raw images. One circular area is marked in a and b. In a to c, light intensity was ˜3 μW/mm².

FIG. 9. All-optical evaluation of pharmacological compounds in M38K cells. Evaluated compounds are PD166866 (PD), AZD6244/Selumetinib (SEL), BIBF1120 (BIBF), UO126 (UO), AP24534/Ponatinib (PON), MK2206 (MK), and LY294002 (LY). Light intensity was ˜3 μW/mm².

FIG. 10. Design and function of mFGFR1-PHY domain chimeric receptor.

(a) RTKs such as mFGFR1 consist of the extracellular ligand-binding domain (LBD), single-span transmembrane domain (TMD) and intracellular domain (ICD) (kinase domain (KD) and a C-terminal tail domain (CTD)). In the mFGFR1-PHY domain fusion protein, only the ICD is retained to render the protein insensitive to endogenous ligand. The ICD is attached to the membrane using a myristoylation domain (MYR) and the PHY domains is incorporated at the ICD C-terminus.

(b) MAPK pathway activation in response to red light for HEK293 cells that were transfected with mFGFR1-SyCP1-PHY, kinase dead mFGFR1-SyCP1-PHY (Y271F, Y272F) or dimerization incompetent mFGFR1-SyCP1-PHY (R195E). Activation is expressed as induction of a luciferase reporter gene. Light intensity was ˜0.05 μW/mm².

FIG. 11. Fusion protein of rtrkB. The chimeric protein of rtrkB-ICD and SyCP1-PHY responds to red light with MAPK pathway activation. Light intensity was ˜0.05 μW/mm².

FIG. 12. All-optical evaluation of pharmacological compounds in HEK293 cells expressing Opto-rtrkB. Evaluated compounds are UO126 (UO), AZD6244/Selumetinib (SEL), PD166866 (PD), Imatinib (IMA), and Vemurafenib/PLX4032 (VEM). For compounds and control (CON), MAPK pathway activation in response to red light was measured and expressed as induction. Light intensity was ˜0.05 μW/mm².

### DESCRIPTION OF THE SEQUENCES

Protein sequences of full length photoreceptors and LOV domains. Uniprot and sequence identifiers are given in parentheses.

AtPH1

(O48963; SEQ ID NO: 1)

MEPTEKPSTKPSSRTLPRDTRGSLEVFNPSTQLTRPDNPVFRPEPPAWQN
LSDPRGTSPQPRPQQEPAPSNPVRSDQEIAVTTSWMALKDPSPETISKKT
ITAEKPQKSAVAAEQRAAEWGLVLKTDTKTGKPQGVGVRNSGGTENDPNG
KKTTSQRNSQNSCRSSGEMSDGDVPGGRSGIPRVSEDLKDALSTFQQTFV
VSDATKPDYPIMYASAGFFNMTGYTSKEVVGRNCRFLQGSGTDADELAKI
RETLAAGNNYCGRILNYKKDGTSFWNLLTIAPIKDESGKVLKFIGMQVEV
SKHTEGAKEKALRPNGLPESLIRYDARQKDMATNSVTELVEAVKRPRALS
ESTNLHPFMTKSESDELPKKPARRMSENWPSGRRNSGGGRRNSMQRINEI
PEKKSRKSSLSFMGIKKKSESLDESIDDGFIEYGEEDDEISDRDERPESV
DDKVRQKEMRKGIDLATTLERIEKNFVITDPRLPDNPIIFASDSFLELTE
YSREEILGRNCRFLQGPETDLTTVKKIRNAIDNQTEVTVQLINYTKSGKK
FWNIFHLQPMRDQKGEVQYFIGVQLDGSKHVEPVRNVIEETAVKEGEDLV
KKTAVNIDEAVRELPDANMTPEDLWANHSKWHCKPHRKDSPPWIAIQKVL
ESGEPIGLKHFKPVKPLGSGDTGSVHLVELVGTDQLFAMKAMDKAVMLNR
NKVHRARAEREILDLLDHPFLPALYASFQTKTHICLITDYYPGGELFMLL
DRQPRKVLKEDAVRFYAAQVVVALEYLHCQGIIYRDLKPENVLIQGNGDI
SLSDFDLSCLTSCKPQLLIPSIDEKKKKKQQKSQQTPIFMAEPMRASNSF
VGTEEYIAPEIISGAGHTSAVDWWALGILMYEMLYGYTPFRGKTRQKTFT
NVLQKDLKFPASIPASLQVKQLIFRLLQRDPKKRLGCFEGANEVKQHSFF
KGINWALIRCTNPPELETPIFSGEAENGEKVVDPELEDLQTNVF
AtPH1-LOV2

(SEQ ID NO: 2)

ESVDDKVRQKEMRKGIDLATTLERIEKNFVITDPRLPDNPIIFASDSFLE
LTEYSREEILGRNCRFLQGPETDLTTVKKIRNAIDNQTEVTVQLINYTKS
GKKFWNIFHLQPMRDQKGEVQYFIGVQLDGSKHVEPVR
AtPH2

(P93025; SEQ ID NO: 3)

MERPRAPPSPLNDAESLSERRSLEIFNPSSGKETHGSTSSSSKPPLDGNN
KGSSSKWMEFQDSAKITERTAEWGLSAVKPDSGDDGISFKLSSEVERSKN
MSRRSSEESTSSESGAFPRVSQELKTALSTLQQTFVVSDATQPHCPIVYA
SSGFFTMTGYSSKEIVGRNCRFLQGPDTDKNEVAKIRDCVKNGKSYCGRL
LNYKKDGTPFWNLLTVTPIKDDQGNTIKFIGMQVEVSKYTEGVNDKALRP
NGLSKSLIRYDARQKEKALDSITEVVQTIRHRKSQVQESVSNDTMVKPDS
STTPTPGRQTRQSDEASKSFRTPGRVSTPTGSKLKSSNNRHEDLLRMEPE
ELMLSTEVIGQRDSWDLSDRERDIRQGIDLATTLERIEKNFVISDPRLPD
NPIIFASDSFLELTEYSREEILGRNCRFLQGPETDQATVQKIRDAIRDQR
EITVQLINYTKSGKKFWNLFHLQPMRDQKGELQYFIGVQLDGSDHVEPLQ
NRLSERTEMQSSKLVKATATNVDEAVRELPDANTRPEDLWAAHSKPVYPL
PHNKESTSWKAIKKIQASGETVGLHHFKPIKPLGSGDTGSVHLVELKGTG
ELYAMKAMEKTMMLNRNKAHRACIEREIISLLDHPFLPTLYASFQTSTHV
CLITDFCPGGELFALLDRQPMKILTEDSARFYAAEVVIGLEYLHCLGIVY
RDLKPENILLKKDGHIVLADFDLSFMTTCTPQLIIPAAPSKRRRSKSQPL
PTFVAEPSTQSNSFVGTEEYIAPEIITGAGHTSAIDWWALGILLYEMLYG
RTPFRGKNRQKTFANILHKDLTFPSSIPVSLVGRQLINTLLNRDPSSRLG
SKGGANEIKQHAFFRGINWPLIRGMSPPPLDAPLSIIEKDPNAKDIKWED
DGVLVNSTDLDIDLF
AtPH2-LOV2

(SEQ ID NO: 4)

DSWDLSDRERDIRQGIDLATTLERIEKNFVISDPRLPDNPIIFASDSFLE
LTEYSREEILGRNCRFLQGPETDQATVQKIRDAIRDQREITVQLINYTKS
GKKFWNLFHLQPMRDQKGELQYFIGVQLDGSDHVEPLQ
CrPH

(A8IXU7; SEQ ID NO: 5)

MAGVPAPASQLTKVLAGLRHTFVVADATLPDCPLVYASEGFYAMTGYGPD
EVLGHNCRFLQGEGTDPKEVQKIRDAIKKGEACSVRLLNYRKDGTPFWNL
LTVTPIKTPDGRVSKFVGVQVDVTSKTEGKALADNSGVPLLVKYDHRLRD
NVARTIVDDVTIAVEKAEGVEPGQASAVAAAAPLGAKGPRGTAPKSFPRV
ALDLATTVERIQQNFCISDPTLPDCPIVFASDAFLELTGYSREEVLGRNC
RFLQGAGTDRGTVDQIRAAIKEGSELTVRILNYTKAGKAFWNMFTLAPMR
DQDGHARFFVGVQVDVTAQSTSPDKAPVWNKTPEEEVAKAKMGAEAASLI
SSALQGMAAPTTANPWAAISGVIMRRKPHKADDKAYQALLQLQERDGKMK
LMHFRRVKQLGAGDVGLVDLVQLQGSELKFAMKTLDKFEMQERNKVARVL
TESAILAAVDHPFLATLYCTIQTDTHLHFVMEYCDGGELYGLLNSQPKKR
LKEEHVRFYASEVLTALQYLHLLGYVYRDLKPENILLHHTGHVLLTDFDL
SYSKGSTTPRIEKIGGAGAAGGSAPKSPKKSSSKSGGSSSGSALQLENYL
LLAEPSARANSFVGTEEYLAPEVINAAGHGPAAVDWWSLGILIFELLYGT
TPFRGARRDETFENIIKSPLKFPSKPAVSEECRDLIEKLLVKDVGARLGS
RTGANEIKSHPWFKGINWALLRHQQPPYVPRRASKAAGGSSTGGAAFDNY
CrPH-LOV1

(SEQ ID NO: 6)

AGLRHTFWADATLPDCPLVYASEGFYAMTGYGPDEVLGHNCRFLQGEGTD
PKEVQKIRDAIKKGEACSVRLLNYRKDGTPFWNLLTVTPIKTPDGRVSKF
VGVQVDVTSKTEGKALA
NcVV

(Q9C3Y6; SEQ ID NO: 7)

MSHTVNSSTMNPWEVEAYQQYHYDPRTAPTANPLFFHTLYAPGGYDIMGY
LIQIMNRPNPQVELGPVDTSCALILCDLKQKDTPIVYASEAFLYMTGYSN
AEVLGRNCRFLQSPDGMVKPKSTRKYVDSNTINTMRKAIDRNAEVQVEVV
NFKKNGQRFVNFLTMIPVRDETGEYRYSMGFQCETE
NcVV-LOV

(SEQ ID NO: 8)

HTLYAPGGYDIMGWLIQIMNRPNPQVELGPVDTSCALILCDLKQKDTPIV
YASEAFLYMTGYSNAEVLGRNCRFLQSPDGMVKPKSTRKYVDSNTINTMR
KAIDRNAEVQVEWNFKKNGQRFVNFLTMIPVRDETGEYRYSMGFQCETE
VfAU1

(A8QW55; SEQ ID NO: 9)

MNGLTPPLMFCSRSDDPSSTSNINLDDVFADVFFNSNGELLDIDEIDDFG
DNTCPKSSMSVDDDASSQVFQGHLFGNALSSIALSDSGDLSTGIYESQGN
ASRGKSLRTKSSGSISSELTEAQKVERRERNREHAKRSRVRKKFLLESLQ
QSVNELNHENNCLKESIREHLGPRGDSLIAQCSPEADTLLTDNPSKANRI
LEDPDYSLVKALQMAQQNFVITDASLPDNPIVYASRGFLTLTGYSLDQIL
GRNCRFLQGPETDPRAVDKIRNAITKGVDTSVCLLNYRQDGTTFWNLFFV
AGLRDSKGNIVNYVGVQSKVSEDYAKLLVNEQNIEYKGVRTSNMLRRK
VfAU1-LOV

(SEQ ID NO: 10)

PDYSLVKALQMAQQNFVITDASLPDNPIVYASRGFLTLTGYSLDQILGRN
CRFLQGPETDPRAVDKIRNAITKGVDTSVCLLNYRQDGTTFWNLFFVAGL
RDSKGNIVNYVGVQSKVSEDYAKLLVNEQNIEYKGVRTSNMLRRK
NgPA1

(K8Z861; SEQ ID NO: 11)

MTEEQKVERRERNREHAKRSRVRKKFLLESLQKSVNALQEENDKLRGAIR
SHLKEGADDLLKTCEVEVDESILASDPCSATKILDDPDYTLVKALQTAQQ
NFVITDPTLPDNPIVYASGGFLSLTGYQMDQILGRNCRFLQGPDTDPAAV
DKIRRAIEDGTDGSVCLLNYRADGSTFWNQFFIAALRGADGNIVNYVGVQ
CKVSEEYASEVLKKEATSSTVAEASSKR
NgPA1-LOV

(SEQ ID NO: 12)

PDYTLVKALQTAQQNFVITDPTLPDNPIVYASGGFLSLTGYQMDQILGRN
CRFLQGPDTDPAAVDKIRRAIEDGTDGSVCLLNYRADGSTFWNQFFIAAL
RGADGNIVNYVGVQCKVSEEYASEVLKKEATSSTVAEASSKR
OdPA1

(C5NSW6; SEQ ID NO: 13)

MTSKQQLPPPPIFGVLGDEKQVARNGIISLVDIFDDFLFSGDRNQPSNTA
SSSSHAQESESVGKDEENDYDSNDDEGDSDDGKRRKRSRTLPRNMTEEQK
IERRERNREHAKRSRVRKKFLLESLQHSVRALEEENEKLRNAIRENLQGE
AEQLLTRCSCGGPSVIASDPNTATRTLDDPDYSLVKALQTAQQNFVISDP
SIPDNPIVYASQGFLTLTGYALSEVLGRNCRFLQGPETDPKAVEKVRKGL
ERGEDTTVVLLNYRKDGSTFWNQLFIAALRDGEGNWNYLGVQCKVSEDYA
KAFLKNEENEK
OdPA1-LOV

(SEQ ID NO: 14)

PDYSLVKALQTAQQNFVISDPSIPDNPIVYASQGFLTLTGYALSEVLGRN
CRFLQGPETDPKAVEKVRKGLERGEDTTVVLLNYRKDGSTFWNQLFIAAL
RDGEGNWNYLGVQCKVSEDYAKAFLKNEENEK
Oligonucleotides utilized in gene construction.
Restriction sites are underlined.
(1) imFGFR1_XhoI_Kozak_F

(SEQ ID NO: 15)

CAGAGCTCGAGACCATGTGGAGCTGGAAGTGCCTCC
(2) imFGFR1_BamHI_R

(SEQ ID NO: 16)

CAGAAGGATCCTCAGCGGCGTTTGAGTCCGCC
(3) imFGFR1_inverse_R

(SEQ ID NO: 17)

TGAGACCGGTCTCGACGCGCCGTTTGAG
(4) imFGFR1_inverse1_F

(SEQ ID NO: 18)

CAAGACCGGTGGATCCGGAGTCGACTATC
(5) imFGFR1_inverse2_F

(SEQ ID NO: 19)

CAAGACCGGTAAACTGGAAGTCGAGGGAGTGC
(6) FKBP_AgeI_F

(SEQ ID NO: 20)

GATCACCGGTAAACTGGAAGTCGAGGGAGTGC
(7) FKBP_XmaI_R

(SEQ ID NO: 21)

GATCCCCGGGACCGCCAGATTCCAGTTTTAGAAG
(8) AtPH1-LOV2_AgeI_F

(SEQ ID NO: 22)

GATCACCGGTGAAAGCGTTGATGATAAGGTCAGACAGAAGG
(9) AtPH1-LOV2_XmaI_R

(SEQ ID NO: 23)

GATCCCCGGGCCGCACGGGCTCAACGTGCT
(10) AtPH2-LOV2_AgeI_F

(SEQ ID NO: 24)

GATCACCGGTGATTCTTGGGATCTGAGTGATAGGGAAAGG
(11) AtPH2-LOV2_XmaI_R

(SEQ ID NO: 25)

GATCCCCGGGCTGGAGTGGCTCGACATGATCTGAC
(12) CrPH1-LOV1_AgeI_F

(SEQ ID NO: 26)

GATCACCGGTGCAGGACTCAGACATACATTTGTGGTGG
(13) CrPH1-LOV1_XmaI_R

(SEQ ID NO: 27)

GATCCCCGGGGGCCAGGGCTTTCCCTTCAGTC
(14) NcVV-LOV_XmaI_F

(SEQ ID NO: 28)

GATCCCCGGGCACACTCTCTACGCCCCAGGCG
(15) NcVV-LOV_XmaI_R

(SEQ ID NO: 29)

GATCCCCGGGTTCGGTTTCGCACTGAAAACCCATGCT
(16) VfAU1-LOV_AgeI_F

(SEQ ID NO: 30)

GATCACCGGTCCTGACTACAGTCTCGTGAAGG
(17) VfAU1-LOV_XmaI_R

(SEQ ID NO: 31)

GATCCCCGGGCTTTCTGCGCAGCATGTTACTGG
(18) Opto-mFGFR1_YY271/2FF_F

(SEQ ID NO: 32)

GAGACATTCATCATATCGACTTCTTCAAGAAAACCACCAACGGCC
(19) Opto-mFGFR1_YY271/2FF_R

(SEQ ID NO: 33)

GGCCGTTGGTGGTTTTCTTGAAGAAGTCGATATGATGAATGTCTC
(20) Opto-mFGFR1_R195E_F

(SEQ ID NO: 34)

TACAGGCCCGGGAGCCTCCTGGGCTGGAGTACTGCTATAA
(21) Opto-mFGFR1_R195E_R

(SEQ ID NO: 35)

TTATAGCAGTACTCCAGCCCAGGAGGCTCCCGGGCCTGTA
(22) Opto-mFGFR1_I472V_F

(SEQ ID NO: 36)

CTCCCAGACAACCCTGTCGTCTACGCCAGTAG
(23) Opto-mFGFR1_I472V_R

(SEQ ID NO: 37)

CTACTGGCGTAGACGACAGGGTTGTCTGGGAG
(24) VfAU1-LOV_BgIII_F

(SEQ ID NO: 38)

CTTTAGATCTCCTGACTACAGTCTCGTGAAGG
(25) VfAU1-LOV_EcoRI_R

(SEQ ID NO: 39)

CTTTGAATTCCTTTCTGCGCAGCATGTTACTG
(26) mFGFR1_inverse_R

(SEQ ID NO: 40)

GATCCACCGGTGACGTCGAGGCGCTGGCTGG
(27) Opto-mFGFR1_inverse_F

(SEQ ID NO: 41)

GATCCACCGGTGGACCTGACTACAGTCTCGTGAAG
(28) imFGFR1_inverse_F

(SEQ ID NO: 42)

GATCCACCGGTGGAAAACTGGAAGTCGAGGGAGTG
(29) hEGFR_AgeI_AscI_F

(SEQ ID NO: 43)

GATCACCGGTGGCGCGCCCGAAGGCGCCACATCGTTC
(30) hEGFR_ICD_BspEI_R

(SEQ ID NO: 44)

GATCTCCGGATGCTCCAATAAATTCACTGCTTTG
(31) hRET_ICD_AgeI_F

(SEQ ID NO: 45)

GATCACCGGTCACTGCTACCACAAGTTTGCC
(32) hRET_ICD_AgeI_R

(SEQ ID NO: 46)

GATCACCGGTGAATCTAGTAAATGCATG
(33) LNGFR_ECD_NotI_F

(SEQ ID NO: 47)

GATCGCGGCCGCACCATGGGGGCAGGTGCCACC
(34) LNGFR_ECD_AscI_R

(SEQ ID NO: 48)

GATCGGCGCGCCC CCTCTTGAAGGCTATGTAGGCC
(35) SyCP1-PHY_F_XmaI

(SEQ ID NO: 61)

GATCCCCGGGGCAACTACTGTTCAACTGTCTGATCAATCTCTG
(36) SyCP1-PHY_R_XmaI

(SEQ ID NO: 62)

GATCCCCGGGTTCTTCAGCTTGGCGCAGAATCAGGTT
(37) redOpto-mFGFR1_inverse_F

(SEQ ID NO: 70)

GATCCACCGGTGGAGCAACTACTGTTCAACTGTCTG
(38) rtrkB_ICD_BspEI_F

(SEQ ID NO: 71)

GATCTCCGGAAAGTTTGGCATGAAAG
(39) rtrkB_ICD_AgeI_R

(SEQ ID NO: 72)

CAGAAACCGGTGCCTAGGATGTCCAG

DNA sequences of codon-optimized LOV domains.

AtPH1-LOV2

(SEQ ID NO: 49)

GAAAGCGTTGATGATAAGGTCAGACAGAAGGAAATGAGAAAGGGAATCGA
TCTCGCAACAACACTCGAAAGAATAGAAAAGAACTTTGTGATTACTGACC
CTAGGCTCCCCGATAATCCCATAATCTTCGCTTCAGACAGTTTCCTGGAG
CTGACAGAGTATAGCCGGGAAGAGATCCTGGGTAGAAATTGCAGATTCCT
GCAGGGACCCGAGACAGACCTGACCACCGTGAAGAAGATTCGCAATGCTA
TCGATAATCAAACCGAGGTTACCGTGCAACTGATAAACTACACTAAAAGC
GGCAAGAAGTTCTGGAACATTTTCCACCTGCAGCCTATGCGGGACCAGAA
GGGTGAGGTCCAATATTTCATCGGGGTGCAGCTGGATGGCAGCAAGCACG
TTGAGCCCGTGCGG
AtPH2-LOV2

(SEQ ID NO: 50)

GATTCTTGGGATCTGAGTGATAGGGAAAGGGATATTAGACAGGGAATAGA
CCTCGCCACCACCCTGGAAAGAATTGAAAAGAATTTCGTGATCAGCGACC
CTAGACTGCCCGACAATCCAATCATTTTCGCCTCTGACTCTTTTCTGGAG
CTGACCGAATACTCACGCGAAGAAATCCTGGGAAGGAACTGTAGGTTCCT
GCAAGGACCCGAAACCGACCAGGCCACTGTCCAGAAGATTCGCGATGCCA
TCCGCGACCAGCGGGAAATTACCGTTCAACTGATCAACTATACCAAATCT
GGTAAGAAGTTTTGGAACCTGTTCCACCTCCAGCCTATGCGGGACCAAAA
GGGCGAACTGCAATATTTCATCGGGGTGCAGCTGGACGGGTCAGATCATG
TCGAGCCACTCCAG
CrPH-LOV1

(SEQ ID NO: 51)

GCAGGACTCAGACATACATTTGTGGTGGCTGATGCAACACTCCCTGATTG
CCCACTGGTCTATGCAAGTGAGGGCTTCTACGCAATGACCGGATATGGAC
CTGACGAAGTGCTGGGTCACAACTGTAGGTTTCTGCAGGGTGAGGGAACT
GACCCCAAGGAAGTGCAGAAAATTCGCGACGCCATCAAGAAGGGTGAGGC
TTGTAGTGTGCGCCTCCTGAACTATCGGAAGGACGGCACTCCCTTCTGGA
ACCTGCTGACAGTCACCCCAATTAAAACCCCTGATGGCCGCGTGTCCAAG
TTTGTCGGCGTGCAGGTGGATGTTACCTCCAAGACTGAAGGGAAAGCCCT
GGCC
NcVV-LOV

(SEQ ID NO: 52)

CACACTCTCTACGCCCCAGGCGGGTACGATATTATGGGCTGGCTGATCCA
GATCATGAACAGGCCCAATCCCCAGGTCGAGCTGGGACCCGTGGATACTT
CATGTGCACTGATACTGTGCGACCTGAAGCAGAAGGATACACCTATAGTT
TACGCTTCAGAAGCCTTTCTGTACATGACAGGGTATTCTAACGCCGAGGT
GCTGGGGAGGAACTGTAGGTTCCTCCAGAGTCCCGATGGTATGGTGAAAC
CTAAGAGTACTCGCAAATATGTGGATAGCAATACTATTAACACCATGAGG
AAAGCCATCGACAGAAACGCAGAAGTTCAGGTGGAAGTGGTGAACTTTAA
GAAGAACGGCCAGCGGTTCGTGAACTTTCTCACAATGATTCCAGTGCGGG
ACGAAACCGGGGAGTACCGGTACAGCATGGGTTTTCAGTGCGAAACCGAA
VfAU1-LOV

(SEQ ID NO: 53)

CCTGACTACAGTCTCGTGAAGGCTCTGCAAATGGCACAACAGAATTTTGT
CATTACAGACGCCTCCCTCCCAGACAACCCTATCGTCTACGCCAGTAGAG
GGTTTCTGACACTGACAGGCTATTCTCTCGACCAGATCCTGGGCAGGAAC
TGCAGGTTTCTGCAAGGGCCAGAAACAGACCCAAGAGCTGTGGATAAGAT
CAGGAATGCCATCACCAAAGGCGTTGATACCAGTGTCTGTCTGCTGAATT
ATAGACAGGATGGCACAACCTTCTGGAATCTCTTCTTCGTGGCTGGACTC
AGAGATTCTAAGGGCAATATTGTCAACTACGTCGGAGTGCAGTCAAAGGT
GAGCGAAGATTATGCCAAGCTGCTGGTCAACGAGCAGAACATTGAGTACA
AAGGTGTGCGCACCAGTAACATGCTGCGCAGAAAG
NgPA1-LOV

(SEQ ID NO: 54)

CCAGATTATACACTCGTTAAAGCACTGCAAACTGCTCAGCAGAATTTTGT
GATCACCGACCCTACTCTGCCAGACAACCCCATTGTCTATGCTTCAGGAG
GATTTCTCAGTCTCACAGGTTACCAGATGGATCAGATCCTGGGAAGAAAT
TGCAGATTTCTGCAAGGACCTGATACTGACCCAGCTGCCGTGGACAAGAT
CAGAAGGGCTATCGAAGATGGTACAGACGGCAGTGTCTGTCTGCTGAACT
ACAGAGCAGATGGATCTACCTTTTGGAATCAATTCTTCATTGCTGCTCTC
AGAGGCGCTGACGGAAATATCGTCAACTATGTCGGAGTGCAGTGTAAAGT
GTCAGAGGAGTATGCTTCAGAAGTCCTCAAGAAGGAGGCTACTTCATCCA
CTGTGGCTGAAGCAAGTAGCAAAAGA
OdPA1-LOV

(SEQ ID NO: 55)

CCTGACTACAGTCTGGTTAAAGCACTCCAAACAGCACAGCAGAATTTCGT
TATCTCTGACCCTAGCATTCCTGATAATCCCATTGTGTATGCTAGTCAGG
GATTTCTGACACTCACCGGATACGCACTGAGCGAGGTTCTCGGACGGAAC
TGCCGGTTCCTCCAAGGACCAGAAACAGACCCTAAAGCCGTCGAGAAAGT
GAGAAAGGGTCTGGAGAGAGGTGAAGATACCACCGTGGTGCTCCTGAATT
ATAGGAAAGATGGAAGCACCTTCTGGAACCAACTGTTCATTGCTGCCCTG
CGGGATGGTGAGGGCAATGTGGTTAACTACCTCGGAGTTCAGTGCAAAGT
CTCCGAGGACTACGCCAAAGCCTTTCTGAAGAATGAAGAGAACGAGAAA

Protein sequences of mFGFR1 variants.

miFGFR1

(SEQ ID NO: 56)

MGSSKSKPKDPSQRLDMKSGTKKSDFHSQMAVHKLAKSIPLRRQVTVSAD
SSASMNSGVLLVRPSRLSSSGTPMLAGVSEYELPEDPRWELPRDRLVLGK
PLGEGCFGQWLAEAIGLDKDKPNRVTKVAVKMLKSDATEKDLSDLISEME
MMKMIGKHKNIINLLGACTQDGPLYVIVEYASKGNLREYLQARRPPGLEY
CYNPSHNPEEQLSSKDLVSCAYQVARGMEYLASKKCIHRDLAARNVLVTE
DNVMKIADFGLARDIHHIDYYKKTTNGRLPVKWMAPEALFDRIYTHQSDV
WSFGVLLWEIFTLGGSPYPGVPVEELFKLLKEGHRMDKPSNCTNELYMMM
RDCWHAVPSQRPTFKQLVEDLDRIVALTSNQEYLDLSIPLDQYSPSFPDT
RSSTCSSGEDSVFSHEPLPEEPCLPRHPTQLANSGLKRRVETGKLEVEGV
QVETISPGDGRTFPKRGQTCWHYTGMLEDGKKVDSSRDRNKPFKFMLGKQ
EVIRGWEEGVAQMSVGQRAKLTISPDYAYGATGHPGIIPPHATLVFDVEL
LKLESGGGSGVDYPYDVPDYALD
miFGFR1-ΔFKBP

(SEQ ID NO: 57)

MGSSKSKPKDPSQRLDMKSGTKKSDFHSQMAVHKLAKSIPLRRQVTVSAD
SSASMNSGVLLVRPSRLSSSGTPMLAGVSEYELPEDPRWELPRDRLVLGK
PLGEGCFGQWLAEAIGLDKDKPNRVTKVAVKMLKSDATEKDLSDLISEME
MMKMIGKHKNIINLLGACTQDGPLYVIVEYASKGNLREYLQARRPPGLEY
CYNPSHNPEEQLSSKDLVSCAYQVARGMEYLASKKCIHRDLAARNVLVTE
DNVMKIADFGLARDIHHIDYYKKTTNGRLPVKWMAPEALFDRIYTHQSDV
WSFGVLLWEIFTLGGSPYPGVPVEELFKLLKEGHRMDKPSNCTNELYMMM
RDCWHAVPSQRPTFKQLVEDLDRIVALTSNQEYLDLSIPLDQYSPSFPDT
RSSTCSSGEDSVFSHEPLPEEPCLPRHPTQLANSGLKRRVETGGSGVDYP
YDVPDYALD
mFGFR1-VfAU1-LOV

(SEQ ID NO: 58)

MGSSKSKPKDPSQRLDMKSGTKKSDFHSQMAVHKLAKSIPLRRQVTVSAD
SSASMNSGVLLVRPSRLSSSGTPMLAGVSEYELPEDPRWELPRDRLVLGK
PLGEGCFGQWLAEAIGLDKDKPNRVTKVAVKMLKSDATEKDLSDLISEME
MMKMIGKHKNIINLLGACTQDGPLYVIVEYASKGNLREYLQARRPPGLEY
CYNPSHNPEEQLSSKDLVSCAYQVARGMEYLASKKCIHRDLAARNVLVTE
DNVMKIADFGLARDIHHIDYYKKTTNGRLPVKWMAPEALFDRIYTHQSDV
WSFGVLLWEIFTLGGSPYPGVPVEELFKLLKEGHRMDKPSNCTNELYMMM
RDCWHAVPSQRPTFKQLVEDLDRIVALTSNQEYLDLSIPLDQYSPSFPDT
RSSTCSSGEDSVFSHEPLPEEPCLPRHPTQLANSGLKRRVETGPDYSLVK
ALQMAQQNFVITDASLPDNPIVYASRGFLTLTGYSLDQILGRNCRFLQGP
ETDPRAVDKIRNAITKGVDTSVCLLNYRQDGTTFWNLFFVAGLRDSKGNI
VNYVGVQSKVSEDYAKLLVNEQNIEYKGVRTSNMLRRKTGGSGVDYPYDV
PDYALD
p75-hEGFR-VfAU1-LOV

(SEQ ID NO: 59)

MGAGATGRAMDGPRLLLLLLLGVSLGGAKEACPTGLYTHSGECCKACNLG
EGVAQPCGANQTVCEPCLDSVTFSDWSATEPCKPCTECVGLQSMSAPCVE
ADDAVCRCAYGYYQDETTGRCEACRVCEAGSGLVFSCQDKQNTVCEECPD
GTYSDEANHVDPCLPCTVCEDTERQLRECTRWADAECEEIPGRWITRSTP
PEGSDSTAPSTQEPEAPPEQDLIASTVAGVVTTVMGSSQPVVTRGTTDNL
IPVYCSILAAVVVGLVAYIAFKRGRARRRHIVRKRTLRRLLQERELVEPL
TPSGEAPNQALLRILKETEFKKIKVLGSGAFGTVYKGLWIPEGEKVKIPV
AIKELREATSPKANKEILDEAYVMASVDNPHVCRLLGICLTSTVQLITQL
MPFGCLLDYVREHKDNIGSQYLLNWCVQIAKGMNYLEDRRLVHRDLAARN
VLVKTPQHVKITDFGLAKLLGAEEKEYHAEGGKVPIKWMALESILHRIYT
HQSDVWSYGVTVWELMTFGSKPYDGIPASEISSILEKGERLPQPPICTID
VYMIMVKCWMIDADSRPKFRELIIEFSKMARDPQRYLVIQGDERMHLPSP
TDSNFYRALMDEEDMDDWDADEYLIPQQGFFSSPSTSRTPLLSSLSATSN
NSTVACIDRNGLQSCPIKEDSFLQRYSSDPTGALTEDSIDDTFLPVPEYI
NQSVPKRPAGSVQNPVYHNQPLNPAPSRDPHYQDPHSTAVGNPEYLNTVQ
PTCVNSTFDSPAHWAQKGSHQISLDNPDYQQDFFPKEAKPNGIFKGSTAE
NAEYLRVAPQSSEFIGASGGPDYSLVKALQMAQQNFVITDASLPDNPIVY
ASRGFLTLTGYSLDQILGRNCRFLQGPETDPRAVDKIRNAITKGVDTSVC
LLNYRQDGTTFWNLFFVAGLRDSKGNIVNYVGVQSKVSEDYAKLLVNEQN
IEYKGVRTSNMLRRKPGGSGVDYPYDVPDYALD
hRET-VfAU1-LOV

(SEQ ID NO: 60)

MGSSKSKPKDPSQRLDVTGHCYHKFAHKPPISSAEMTFRRPAQAFPVSYS
SSSARRPSLDSMENQVSVDAFKILEDPKWEFPRKNLVLGKTLGEGEFGKV
VKATAFHLKGRAGYTTVAVKMLKENASPSELRDLLSEFNVLKQVNHPHVI
KLYGACSQDGPLLLIVEYAKYGSLRGFLRESRKVGPGYLGSGGSRNSSSL
DHPDERALTMGDLISFAWQISQGMQYLAEMKLVHRDLAARNILVAEGRKM
KISDFGLSRDVYEEDSYVKRSQGRIPVKWMAIESLFDHIYTTQSDVWSFG
VLLWEIVTLGGNPYPGIPPERLFNLLKTGHRMERPDNCSEEMYRLMLQCW
KQEPDKRPVFADISKDLEKMMVKRRDYLDLAASTPSDSLIYDDGLSEEET
PLVDCNNAPLPRALPSTWIENKLYGRISHAFTRFTGGPDYSLVKALQMAQ
QNFVITDASLPDNPIVYASRGFLTLTGYSLDQILGRNCRFLQGPETDPRA
VDKIRNAITKGVDTSVCLLNYRQDGTTFWNLFFVAGLRDSKGNIVNYVGV
QSKVSEDYAKLLVNEQNIEYKGVRTSNMLRRKPGGSGVDYPYDVPDYALD

### Percent Identity Tables

| | VfAU1-LOV | NgPA1-LOV | OdPA1-LOV |
|---|---|---|---|
| VfAU1-LOV | 100% | 75% | 73% |
| NgPA1-LOV | 75% | 100% | 73% |
| OdPA1-LOV | 73% | 73% | 100% |

Protein sequences of full length proteins and PHY domain. Uniprot identifiers are given in parentheses.

SyCP1 (Q55168) (SEQ ID NO: 63):
MATTVQLSDQSLRQLETLAIHTAHLIQPHGLVWLQEPDLTISQISANCTG
ILGRSPEDLLGRTLGEVFDSFQIDPIQSRLTAGQISSLNPSKLWARVMGD
DFVIFDGVFHRNSDGLLVCELEPAYTSDNLPFLGFYHMANAALNRLRQQA
NLRDFYDVIVEEVRRMTGFDRVMLYRFDENNHGDVIAEDKRDDMEPYLGL
HYPESDIPQPARRLFIHNPIRVIPDVYGVAVPLTPAVNPSTNRAVDLTES
ILRSAYHCHLTYLKNMGVGASLTISLIKDGHLWGLIACHHQTPKVIPFEL
RKACEFFGRWFSNISAQEDTETFDYRVQLAEHEAVLLDKMTTAADFVEGL
TNHPDRLLGLTGSQGAAICFGEKLILVGETPDEKAVQYLLQWLENREVQD
VFFTSSLSQIYPDAVNFKSVASGLLAIPIARHNFLLWFRPEVLQTVNWGG
DPNHAYEATQEDGKIELHPRQSFDLWKEIVRLQSLPWQSVEIQSALALKK
AIVNLILRQAEELAQLARNLERSNADLKKFAYIASHDLQEPLNQVSNYVQ
LLEMRYSEALDEDAKDFIDFAVTGVSLMQTLIDDILTYAKVDTQYAQLTF
TDVQEVVDKALANLKQRIEESGAEIEVGSMPAVMADQIQLMQVFQNLIAN
GIKFAGDKSPKIKIWGDRQEDAWVFAVQDNGIGIDPQFFERIFVIFQRLH
TRDEYKGTGMGLAICKKIIEGHQGQIWLESNPGEGSTFYFSIPIGN
SyCP1-PHY (SEQ ID NO: 64):
ATTVQLSDQSLRQLETLAIHTAHLIQPHGLVWLQEPDLTISQISANCTGI
LGRSPEDLLGRTLGEVFDSFQIDPIQSRLTAGQISSLNPSKLWARVMGDD
FVIFDGVFHRNSDGLLVCELEPAYTSDNLPFLGFYHMANAALNRLRQQAN
LRDFYDVIVEEVRRMTGFDRVMLYRFDENNHGDVIAEDKRDDMEPYLGLH
YPESDIPQPARRLFIHNPIRVIPDVYGVAVPLTPAVNPSTNRAVDLTESI
LRSAYHCHLTYLKNMGVGASLTISLIKDGHLWGLIACHHQTPKVIPFELR
KACEFFGRWFSNISAQEDTETFDYRVQLAEHEAVLLDKMTTAADFVEGLT
NHPDRLLGLTGSQGAAICFGEKLILVGETPDEKAVQYLLQWLENREVQDV
FFTSSLSQIYPDAVNFKSVASGLLAIPIARHNFLLWFRPEVLQTVNWGGD
PNHAYEATQEDGKIELHPRQSFDLWKEIVRLQSLPWQSVEIQSALALKKA
IVNLILRQAEE

DNA sequences of codon-optimized PHY domain.

SyCP1-PHY (SEQ ID NO: 65):
GCAACTACTGTTCAACTGTCTGATCAATCTCTGCGTCAACTGGAAACTCT
GGCTATCCACACCGCGCATCTGATCCAGCCGCACGGTCTGGTAGTCGTCC
TGCAAGAACCGGACCTGACCATCAGCCAGATCTCTGCGAACTGTACCGGT
ATCCTGGGCCGTAGCCCGGAAGATCTGCTGGGTCGTACTCTGGGCGAGGT
ATTCGATTCTTTTCAGATTGATCCGATCCAGTCTCGTCTGACCGCAGGTC
AGATTTCCAGCCTGAACCCGTCCAAGCTGTGGGCGCGTGTTATGGGTGAC
GACTTTGTTATTTTCGACGGCGTATTTCATCGTAACTCTGATGGCCTGCT
GGTTTGCGAGCTGGAGCCGGCCTACACTAGCGACAACCTGCCTTTCCTGG
GTTTCTACCATATGGCAAACGCGGCACTGAACCGTCTGCGTCAGCAAGCT
AACCTGCGCGACTTCTACGACGTTATCGTTGAGGAAGTGCGCCGCATGAC
GGGTTTCGACCGCGTCATGCTGTACCGTTTTGATGAAAACAACCACGGTG
ACGTAATCGCGGAGGATAAGCGTGACGACATGGAGCCGTATCTGGGTCTG
CACTACCCGGAAAGCGACATTCCTCAGCCGGCACGTCGCCTGTTCATTCA
CAACCCGATCCGTGTTATTCCGGACGTTTACGGCGTTGCTGTTCCGCTGA
CTCCGGCCGTTAATCCGTCTACTAACCGTGCAGTTGACCTGACCGAATCC
ATCCTGCGTTCCGCATACCATTGCCACCTGACCTATCTGAAGAACATGGG
CGTTGGTGCTAGCCTGACGATCTCTCTGATTAAAGATGGTCACCTGTGGG
GTCTGATCGCTTGCCATCACCAGACCCCGAAAGTAATCCCTTTCGAACTG
CGTAAAGCCTGCGAATTCTTCGGTCGTGTGGTGTTCTCTAATATCTCCGC
GCAAGAAGACACCGAGACTTTTGACTACCGCGTACAGCTGGCGGAGCATG
AAGCGGTTCTGCTGGACAAAATGACCACCGCGGCAGACTTCGTGGAGGGC
CTGACTAACCACCCAGACCGTCTGCTGGGCCTGACCGGCAGCCAAGGCGC
TGCGATTTGTTTCGGCGAGAAACTGATTCTGGTGGGCGAAACCCCAGACG
AAAAGGCGGTGCAATACCTGCTGCAATGGCTGGAGAATCGCGAAGTGCAG
GACGTTTTCTTCACTAGCTCTCTGTCTCAGATCTATCCGGATGCGGTTAA
CTTCAAAAGCGTGGCGTCCGGCCTGCTGGCTATCCCGATCGCCCGTCATA
ACTTTCTGCTGTGGTTCCGCCCGGAGGTTCTGCAGACCGTTAATTGGGGT
GGTGATCCGAATCACGCATACGAAGCAACCCAAGAAGATGGTAAGATCGA
ACTGCATCCGCGTCAGTCCTTCGATCTGTGGAAAGAAATTGTTCGCCTGC
AGAGCCTGCCGTGGCAGAGCGTTGAGATCCAGTCTGCCCTGGCTCTGAAG
AAAGCAATCGTGAACCTGATTCTGCGCCAAGCTGAAGAA

Protein sequences of full length fusion proteins.

redOpto-mFGFR1

(SEQ ID NO: 66)

MGSSKSKPKDPSQRLDMKSGTKKSDFHSQMAVHKLAKSIPLRRQVTVSAD
SSASMNSGVLLVRPSRLSSSGTPMLAGVSEYELPEDPRWELPRDRLVLGK
PLGEGCFGQWLAEAIGLDKDKPNRVTKVAVKMLKSDATEKDLSDLISEME
MMKMIGKHKNIINLLGACTQDGPLYVIVEYASKGNLREYLQARRPPGLEY
CYNPSHNPEEQLSSKDLVSCAYQVARGMEYLASKKCIHRDLAARNVLVTE
DNVMKIADFGLARDIHHIDYYKKTTNGRLPVKWMAPEALFDRIYTHQSDV
WSFGVLLWEIFTLGGSPYPGVPVEELFKLLKEGHRMDKPSNCTNELYMMM
RDCWHAVPSQRPTFKQLVEDLDRIVALTSNQEYLDLSIPLDQYSPSFPDT
RSSTCSSGEDSVFSHEPLPEEPCLPRHPTQLANSGLKRRVETGATTVQLS
DQSLRQLETLAIHTAHLIQPHGLVVVLQEPDLTISQISANCTGILGRSPE
DLLGRTLGEVFDSFQIDPIQSRLTAGQISSLNPSKLWARVMGDDFVIFDG
VFHRNSDGLLVCELEPAYTSDNLPFLGFYHMANAALNRLRQQANLRDFYD
VIVEEVRRMTGFDRVMLYRFDENNHGDVIAEDKRDDMEPYLGLHYPESDI
PQPARRLFIHNPIRVIPDVYGVAVPLTPAVNPSTNRAVDLTESILRSAYH
CHLTYLKNMGVGASLTISLIKDGHLWGLIACHHQTPKVIPFELRKACEFF
GRWFSNISAQEDTETFDYRVQLAEHEAVLLDKMTTAADFVEGLTNHPDRL
LGLTGSQGAAICFGEKLILVGETPDEKAVQYLLQWLENREVQDVFFTSSL
SQIYPDAVNFKSVASGLLAIPIARHNFLLWFRPEVLQTVNWGGDPNHAYE
ATQEDGKIELHPRQSFDLWKEIVRLQSLPWQSVEIQSALALKKAIVNLIL
RQAEETGGSGVDYPYDVPDYALD
redOpto-rtrkB

(SEQ ID NO: 67)

MGSSKSKPKDPSQRLDVTGKLARHSKFGMKGPASVISNDDDSASPLHHIS
NGSNTPSSSEGGPDAVIIGMTKIPVIENPQYFGITNSQLKPDTFVQHIKR
HNIVLKRELGEGAFGKVFLAECYNLCPEQDKILVAVKTLKDASDNARKDF
HREAELLTNLQHEHIVKFYGVCVEGDPLIMVFEYMKHGDLNKFLRAHGPD
AVLMAEGNPPTELTQSQMLHIAQQIAAGMVYLASQHFVHRDLATRNCLVG
ENLLVKIGDFGMSRDVYSTDYYRVGGHTMLPIRWMPPESIMYRKFTTESD
VWSLGVVLWEIFTYGKQPWYQLSNNEVIECITQGRVLQRPRTCPQEVYEL
MLGCWQREPHTRKNIKNIHTLLQNLAKASPVYLDILGTGGATTVQLSDQS
LRQLETLAIHTAHLIQPHGLVVVLQEPDLTISQISANCTGILGRSPEDLL
GRTLGEVFDSFQIDPIQSRLTAGQISSLNPSKLWARVMGDDFVIFDGVFH
RNSDGLLVCELEPAYTSDNLPFLGFYHMANAALNRLRQQANLRDFYDVIV
EEVRRMTGFDRVMLYRFDENNHGDVIAEDKRDDMEPYLGLHYPESDIPQP
ARRLFIHNPIRVIPDVYGVAVPLTPAVNPSTNRAVDLTESILRSAYHCHL
TYLKNMGVGASLTISLIKDGHLWGLIACHHQTPKVIPFELRKACEFFGRV
VFSNISAQEDTETFDYRVQLAEHEAVLLDKMTTAADFVEGLTNHPDRLLG
LTGSQGAAICFGEKLILVGETPDEKAVQYLLQWLENREVQDVFFTSSLSQ
IYPDAVNFKSVASGLLAIPIARHNFLLWFRPEVLQTVNWGGDPNHAYEAT
QEDGKIELHPRQSFDLWKEIVRLQSLPWQSVEIQSALALKKAIVNLILRQ
AEEPGGSGVDYPYDVPDYALD

DNA sequences of full length fusion proteins.

redOpto-mFGFR1

(SEQ ID NO: 68)

ATGGGGAGTAGCAAGAGCAAGCCTAAGGACCCCAGCCAGCGCCTCGACAT
GAAGAGCGGCACCAAGAAGAGCGACTTCCATAGCCAGATGGCTGTGCACA
AGCTGGCCAAGAGCATCCCTCTGCGCAGACAGGTAACAGTGTCAGCTGAC
TCCAGTGCATCCATGAACTCTGGGGTTCTCCTGGTTCGGCCCTCACGGCT
CTCCTCCAGCGGGACCCCCATGCTGGCTGGAGTCTCCGAATATGAGCTCC
CTGAGGATCCCCGCTGGGAGCTGCCACGAGACAGACTGGTCTTAGGCAAA
CCACTTGGCGAGGGCTGCTTCGGGCAGGTGGTGTTGGCTGAGGCCATCGG
GCTGGATAAGGACAAACCCAACCGTGTGACCAAAGTGGCCGTGAAGATGT
TGAAGTCCGACGCAACGGAGAAGGACCTGTCGGATCTGATCTCGGAGATG
GAGATGATGAAAATGATTGGGAAGCACAAGAATATCATCAACCTTCTGGG
AGCGTGCACACAGGATGGTCCTCTTTATGTCATTGTGGAGTACGCCTCCA
AAGGCAATCTCCGGGAGTATCTACAGGCCCGGAGGCCTCCTGGGCTGGAG
TACTGCTATAACCCCAGCCACAACCCCGAGGAACAGCTGTCTTCCAAAGA
TCTGGTATCCTGTGCCTATCAGGTGGCTCGGGGCATGGAGTATCTTGCCT
CTAAGAAGTGTATACACCGAGACCTGGCTGCTAGGAACGTCCTGGTGACC
GAGGATAACGTAATGAAGATCGCAGACTTTGGCTTAGCTCGAGACATTCA
TCATATCGACTACTACAAGAAAACCACCAACGGCCGGCTGCCTGTGAAGT
GGATGGCCCCTGAGGCGTTGTTTGACCGGATCTACACACACCAGAGCGAT
GTGTGGTCTTTTGGAGTGCTCTTGTGGGAGATCTTCACTCTGGGTGGCTC
CCCATACCCCGGTGTGCCTGTGGAGGAACTTTTCAAGCTGCTGAAGGAGG
GTCATCGAATGGACAAGCCCAGTAACTGTACCAATGAGCTGTACATGATG
ATGCGGGACTGCTGGCATGCAGTGCCCTCTCAGAGACCTACGTTCAAGCA
GTTGGTGGAAGACCTGGACCGCATTGTGGCCTTGACCTCCAACCAGGAGT
ATCTGGACCTGTCCATACCGCTGGACCAGTACTCACCCAGCTTTCCCGAC
ACACGGAGCTCCACCTGCTCCTCAGGGGAGGACTCTGTCTTCTCTCATGA
GCCGTTACCTGAGGAGCCCTGTCTGCCTCGACACCCCACCCAGCTTGCCA
ACAGTGGACTCAAACGGCGCGTCGAGACCGGgGCAACTACTGTTCAACTG
TCTGATCAATCTCTGCGTCAACTGGAAACTCTGGCTATCCACACCGCGCA
TCTGATCCAGCCGCACGGTCTGGTAGTCGTCCTGCAAGAACCGGACCTGA
CCATCAGCCAGATCTCTGCGAACTGTACCGGTATCCTGGGCCGTAGCCCG
GAAGATCTGCTGGGTCGTACTCTGGGCGAGGTATTCGATTCTTTTCAGAT
TGATCCGATCCAGTCTCGTCTGACCGCAGGTCAGATTTCCAGCCTGAACC
CGTCCAAGCTGTGGGCGCGTGTTATGGGTGACGACTTTGTTATTTTCGAC
GGCGTATTTCATCGTAACTCTGATGGCCTGCTGGTTTGCGAGCTGGAGCC
GGCCTACACTAGCGACAACCTGCCTTTCCTGGGTTTCTACCATATGGCAA
ACGCGGCACTGAACCGTCTGCGTCAGCAAGCTAACCTGCGCGACTTCTAC
GACGTTATCGTTGAGGAAGTGCGCCGCATGACGGGTTTCGACCGCGTCAT
GCTGTACCGTTTTGATGAAAACAACCACGGTGACGTAATCGCGGAGGATA
AGCGTGACGACATGGAGCCGTATCTGGGTCTGCACTACCCGGAAAGCGAC
ATTCCTCAGCCGGCACGTCGCCTGTTCATTCACAACCCGATCCGTGTTAT
TCCGGACGTTTACGGCGTTGCTGTTCCGCTGACTCCGGCCGTTAATCCGT
CTACTAACCGTGCAGTTGACCTGACCGAATCCATCCTGCGTTCCGCATAC
CATTGCCACCTGACCTATCTGAAGAACATGGGCGTTGGTGCTAGCCTGAC
GATCTCTCTGATTAAAGATGGTCACCTGTGGGGTCTGATCGCTTGCCATC
ACCAGACCCCGAAAGTAATCCCTTTCGAACTGCGTAAAGCCTGCGAATTC
TTCGGTCGTGTGGTGTTCTCTAATATCTCCGCGCAAGAAGACACCGAGAC
TTTTGACTACCGCGTACAGCTGGCGGAGCATGAAGCGGTTCTGCTGGACA
AAATGACCACCGCGGCAGACTTCGTGGAGGGCCTGACTAACCACCCAGAC
CGTCTGCTGGGCCTGACCGGCAGCCAAGGCGCTGCGATTTGTTTCGGCGA
GAAACTGATTCTGGTGGGCGAAACCCCAGACGAAAAGGCGGTGCAATACC
TGCTGCAATGGCTGGAGAATCGCGAAGTGCAGGACGTTTTCTTCACTAGC
TCTCTGTCTCAGATCTATCCGGATGCGGTTAACTTCAAAAGCGTGGCGTC
CGGCCTGCTGGCTATCCCGATCGCCCGTCATAACTTTCTGCTGTGGTTCC
GCCCGGAGGTTCTGCAGACCGTTAATTGGGGTGGTGATCCGAATCACGCA
TACGAAGCAACCCAAGAAGATGGTAAGATCGAACTGCATCCGCGTCAGTC
CTTCGATCTGTGGAAAGAAATTGTTCGCCTGCAGAGCCTGCCGTGGCAGA
GCGTTGAGATCCAGTCTGCCCTGGCTCTGAAGAAAGCAATCGTGAACCTG
ATTCTGCGCCAAGCTGAAGAAcCCGGTGGATCCGGAGTCGACTATCCGTA
CGACGTACCAGACTACGCACTCGACTAA
redOpto-rtrkB

(SEQ ID NO: 69)

ATGGGGAGTAGCAAGAGCAAGCCTAAGGACCCCAGCCAGCGCCTCGACGT
CACCGGAAAGTTGGCGAGACATTCCAAGTTTGGCATGAAAGGCCCAGCTT
CCGTCATCAGCAACGACGATGACTCTGCCAGCCCTCTCCACCACATCTCC
AACGGGAGCAACACTCCGTCTTCTTCGGAGGGCGGGCCCGATGCTGTCAT
CATTGGGATGACCAAGATCCCTGTCATTGAAAACCCCCAGTACTTCGGTA
TCACCAACAGCCAGCTCAAGCCGGACACATTTGTTCAGCACATCAAGAGA
CACAACATCGTTCTGAAGAGGGAGCTTGGAGAAGGAGCCTTTGGGAAAGT
TTTCCTAGCGGAGTGCTATAACCTCTGCCCCGAGCAGGATAAGATCCTGG
TGGCCGTGAAGACGCTGAAGGACGCCAGCGACAATGCTCGCAAGGACTTT
CATCGCGAAGCCGAGCTGCTGACCAACCTCCAGCACGAGCACATTGTCAA
GTTCTACGGTGTCTGTGTGGAGGGCGACCCACTCATCATGGTCTTTGAGT
ACATGAAGCACGGGGACCTCAACAAGTTCCTTAGGGCACACGGGCCAGAT
GCAGTGCTGATGGCAGAGGGTAACCCGCCCACCGAGCTGACGCAGTCGCA
GATGCTGCACATCGCTCAGCAAATCGCAGCAGGCATGGTCTACCTGGCAT
CCCAACACTTCGTGCACCGAGACCTGGCCACCCGGAACTGCTTGGTAGGA
GAGAACCTGCTGGTGAAAATTGGGGACTTCGGGATGTCCCGGGATGTATA
CAGCACCGACTACTACCGGGTTGGTGGCCACACAATGTTGCCCATCCGAT
GGATGCCTCCAGAGAGCATCATGTACAGGAAATTCACCACCGAGAGTGAC
GTCTGGAGCCTGGGAGTTGTGTTGTGGGAGATCTTCACCTACGGCAAGCA
GCCCTGGTATCAGCTATCAAACAACGAGGTGATAGAATGCATCACCCAGG
GCAGAGTCCTTCAGCGGCCTCGCACGTGTCCCCAGGAGGTGTACGAGCTG
ATGCTGGGATGCTGGCAGCGGGAACCACACACAAGGAAGAACATCAAGAA
CATCCACACACTCCTTCAGAACTTGGCGAAGGCGTCGCCCGTCTACCTGG
ACATCCTAGGCACCGGTGGAGCAACTACTGTTCAACTGTCTGATCAATCT
CTGCGTCAACTGGAAACTCTGGCTATCCACACCGCGCATCTGATCCAGCC
GCACGGTCTGGTAGTCGTCCTGCAAGAACCGGACCTGACCATCAGCCAGA
TCTCTGCGAACTGTACCGGTATCCTGGGCCGTAGCCCGGAAGATCTGCTG
GGTCGTACTCTGGGCGAGGTATTCGATTCTTTTCAGATTGATCCGATCCA
GTCTCGTCTGACCGCAGGTCAGATTTCCAGCCTGAACCCGTCCAAGCTGT
GGGCGCGTGTTATGGGTGACGACTTTGTTATTTTCGACGGCGTATTTCAT
CGTAACTCTGATGGCCTGCTGGTTTGCGAGCTGGAGCCGGCCTACACTAG
CGACAACCTGCCTTTCCTGGGTTTCTACCATATGGCAAACGCGGCACTGA
ACCGTCTGCGTCAGCAAGCTAACCTGCGCGACTTCTACGACGTTATCGTT
GAGGAAGTGCGCCGCATGACGGGTTTCGACCGCGTCATGCTGTACCGTTT
TGATGAAAACAACCACGGTGACGTAATCGCGGAGGATAAGCGTGACGACA
TGGAGCCGTATCTGGGTCTGCACTACCCGGAAAGCGACATTCCTCAGCCG
GCACGTCGCCTGTTCATTCACAACCCGATCCGTGTTATTCCGGACGTTTA
CGGCGTTGCTGTTCCGCTGACTCCGGCCGTTAATCCGTCTACTAACCGTG
CAGTTGACCTGACCGAATCCATCCTGCGTTCCGCATACCATTGCCACCTG
ACCTATCTGAAGAACATGGGCGTTGGTGCTAGCCTGACGATCTCTCTGAT
TAAAGATGGTCACCTGTGGGGTCTGATCGCTTGCCATCACCAGACCCCGA
AAGTAATCCCTTTCGAACTGCGTAAAGCCTGCGAATTCTTCGGTCGTGTG
GTGTTCTCTAATATCTCCGCGCAAGAAGACACCGAGACTTTTGACTACCG
CGTACAGCTGGCGGAGCATGAAGCGGTTCTGCTGGACAAAATGACCACCG
CGGCAGACTTCGTGGAGGGCCTGACTAACCACCCAGACCGTCTGCTGGGC
CTGACCGGCAGCCAAGGCGCTGCGATTTGTTTCGGCGAGAAACTGATTCT
GGTGGGCGAAACCCCAGACGAAAAGGCGGTGCAATACCTGCTGCAATGGC
TGGAGAATCGCGAAGTGCAGGACGTTTTCTTCACTAGCTCTCTGTCTCAG
ATCTATCCGGATGCGGTTAACTTCAAAAGCGTGGCGTCCGGCCTGCTGGC
TATCCCGATCGCCCGTCATAACTTTCTGCTGTGGTTCCGCCCGGAGGTTC
TGCAGACCGTTAATTGGGGTGGTGATCCGAATCACGCATACGAAGCAACC
CAAGAAGATGGTAAGATCGAACTGCATCCGCGTCAGTCCTTCGATCTGTG
GAAAGAAATTGTTCGCCTGCAGAGCCTGCCGTGGCAGAGCGTTGAGATCC
AGTCTGCCCTGGCTCTGAAGAAAGCAATCGTGAACCTGATTCTGCGCCAA
GCTGAAGAAcCCGGTGGATCCGGAGTCGACTATCCGTACGACGTACCAGA
CTACGCACTCGACTAA

### EXAMPLES

### Materials and Methods

mFGFR1 Receptor Constructs

pSH1/M-FGFR1-Fv-Fvls-E (D. M. Spencer, Baylor College of Medicine; (Welm, Freeman et al. 2002)) was obtained from Addgene (Cambridge, Mass.). The intracellular fragment of mFGFR1 flanked by a myristoylation domain, two FKBP domains and an hemagglutinin epitope was transferred from pSH1/M-FGFR1-Fv-Fvls-E to pcDNA3.1(−) (Invitrogen/LifeTech, Vienna, Austria) using PCR and XhoI and BamHI restriction enzymes (oligonucleotides (1) and (2), SEQ ID NOs: 15 and 16). Using inverse PCR, a single or both FKBP domains were deleted to yield constructs miFGFR1 and miFGFR1-ΔFKBP. In this reaction, amplification using oligonucleotides 3 and 4 or 3 and 5 (SEQ ID NOs: 17, 18, and 19, respectively) produced linear DNA fragments in which either both or one FKBP domain was replaced by terminal AgeI restriction sites. Linear products were digested with AgeI, ligated and directly transformed into E. coli bacteria for production. This reaction also introduced the AgeI restriction site in miFGFR1-ΔFKBP that was used for LOV domain insertion (see below). As an additional control, one FKBP domain was re-inserted in miFGFR1-ΔFKBP using PCR and AgeI and XmaI restriction enzymes (oligonucleotides 6 and 7; SEQ ID NOs: 20 and 21). miFGFR1 and miFGFR1-ΔFKBP-FKBP produced similar results in MAPK activation assays and were used interchangeably. All constructs were verified by DNA sequencing.

LOV Domains and Chimeric mFGFR1 Receptors

Genes coding for the LOV domains of A. thaliana phototropin 1 (AtPH1-LOV2, residue 449 to 586 of Uniprot sequence O48963), A. thaliana phototropin 2 (AtPH2-LOV2, residue 363 to 500 of Uniprot sequence P93025), C. reinhardtii phototropin (CrPH-LOV1, residue 16 to 133 of Uniprot sequence A8IXU7), N. crassa vivid (NcVV-LOV, residue 37 to 186 with Y50W mutation of Uniprot sequence Q9C3Y6), V. frigida aureochrome1 (VfAU1-LOV, residue 204 to 348 of Uniprot sequence A8QW55), N. gaditana hypothetical protein NGA_0015702 (NgPA1-LOV, residue 87 to 228 of Uniprot sequence K8Z861) and O. danica aureochrome1-like protein (OdPA1-LOV, residue 180 to 312 of Uniprot sequence C5NSW6) were synthesized with mammalian codon optimization according to the supplier's recommendation (Epoch Life Science, Inc., Missouri City, Tex., USA) (see also SEQ ID NOs: 49-55). NgPA1-LOV and OdPA1-LOV were identified using database searches for proteins with similarity to VfAU1 from the non-redundant protein database of the National Center for Biotechnology Information. LOV domains were inserted into miFGFR1-ΔFKBP using PCR and AgeI and XmaI restriction enzymes (oligonucleotides 8 to 17, SEQ ID NOs: 22-31; NgPA1-LOV and OdPA1-LOV were synthesized with restriction sites and inserted without PCR). All constructs were verified by DNA sequencing.

### Modified Opto-mFGFR1 Receptors

Point substitutions (YY271FF, R192E and I472V; numbered relative to start methionine of Opto-mFGFR1) were introduced in Opto-mFGFR1 or redOpto-mFGFR1 using site-directed mutagenesis (QuickChangeII Site-Directed Mutagenesis Kit, Agilent, Vienna, Austria; oligonucleotides 18 to 23) (SEQ ID NOs: 32-37). All constructs were verified by DNA sequencing.

### Light-Activated VfAU1-LOV Transcription Factor

The plasmid pGAVPO (Y. Yang, East China University of Science and Technology) contains a Gal4 DNA binding domain, NcVV-LOV and a transactivation domain (Wang et al. 2012) (FIG. 4b). Blue-light activation of pGAVPO was detected with the luciferase reporter plasmid applied in MAPK pathways assays (see above; this plasmid contains multiple UAS sequences). VfAU1-LOV was amplified by PCR and oligonucleotides 24 and 25 (SEQ ID NO: 38 and 39, respectively) and inserted in pGAVPO using BglII and EcoRI restriction enzymes. Constructs was verified by DNA sequencing. Luciferase activation experiments were performed as described above except that mFGFR1 plasmids were replaced with 50 ng pGAVPO per well.

### Opto-hEGFR1 and Opto-hRET

Using inverse PCR, expression plasmids were prepared based on imFGFR1-ΔFKBP-FKBP and mFGFR1-VfAU1-LOV in which the mFGFR1 ICD was replaced by a SgrAI-restriction site (oligonucleotides 26-28, SEQ ID NOs: 40-42). This single restriction site allows inserting ICDs of other RTKs. hEGFR ICD and hRET ICD were inserted into this plasmid using PCR and AgeI and BspEI restriction enzymes (oligonucleotides 29-32, SEQ ID NOs: 43-46). The EGFR construct was further modified by including the LBD and TMD of p75 using PCR and NotI and AscI restriction enzymes (oligonucleotides 33 and 34, SEQ ID NOs: 47 and 48). All constructs were verified by DNA sequencing.

PHY Domain and Chimeric mFGFR1 Receptors

A gene coding for the PHY domain of Synechocystis PCC6803 CPH1 (SyCP1-PHY, residue 2 to 514 of Uniprot sequence Q55168) were synthesized with mammalian codon optimization according to the supplier's recommendation (Epoch Life Science, Inc., Missouri City, Tex., USA) (SEQ ID NO: 63-65). PHY domain was inserted into imFGFR1-ΔFKBP using PCR the XmaI restriction enzymes (oligonucleotides 35 and 36; SEQ ID NO: 61 and 62). The construct was verified by DNA sequencing and termed redOpto-mFGFR1.

redOpto-rtrkB

Using inverse PCR, an expression plasmid was prepared based on redOpto-mFGFR1 in which the mFGFR1 ICD was replaced by a SgrAI-restriction site (oligonucleotides 26 and 37, SEQ ID NOs: 40 and 70). This single restriction site allows inserting ICDs of other RTKs. rtrkB ICD was inserted into this plasmid using PCR and AgeI and BspEI restriction enzymes (oligonucleotides 38 and 39, SEQ ID NOs: 71 and 72).

### Custom Incubator for Light Stimulation of Cells

For light stimulation of cells, an incubator (PT2499, ExoTerra/HAGEN, Holm, Germany) was equipped with 300 light emitting diodes (JS-FS505ORGB-W30 with JS-CON-004 controller, Komerci, Ebern, Germany; λₘₐₓ˜630 nm (red), λₘₐₓ˜530 nm (green), λₘₐₓ˜470 nm (blue), bandwidth ˜±5 nm). Light intensity was controlled with an analog dimmer and measured with a digital power meter (PM120VA, Thorlabs, Munich, Germany). Intensities at maximal output were 2.3 (red), 2.6 (green) and 3.3 (blue light) W/m². For stimulation over extended time periods (>8 h), an aluminium box was equipped with the same light-emitting diodes and controller and placed in an incubator with standard tissue culture conditions (see below).

### Cell Culture and Transfection (HEK293 and CHO-K1 Cells)

HEK293 cells and CHO-K1 (American Type Culture Collection (ATCC), Manassas, Va.) cells were maintained in DMEM supplemented with 10% FBS, 100 U/ml penicilin and 0.1 mg/ml streptomycin in a humidified incubator with 5% CO2 atmosphere. After trypsination, 5×10⁴ cells were seeded in each well of 96-well plates (three to four wells for each construct) coated with poly-L-ornithine (Sigma, Vienna, Austria). Either transparent plates or black clear bottom plates were used. Cells were transfected using Lipofectamine 2000 (Invitrogen/LifeTech).

### Phycocyanobilin Incubation

Phycocyanobilin (PCB; Livchem Logistics GmbH, Frankfurt a.M., Germany) was dissolved in a dark room to a stock concentration of 10 mM in DMSO. Aliquots were stored in the dark at −20° C. Prior to experiments, cells were incubated with 50 μM PCB overnight in reduced serum starve medium at 37° C. PCB could also be applied to applied to living animals as it is non-toxic even in high doses (0.17% of diet or 10 mg/kg) (McCarty (2007).

### LOV Domain Expression and Cell Proliferation Measurements (HEK293 and CHO-K1 Cells)

An expression plasmid based on pcDNA3.1(−) was prepared in which a BspEI-restriction site followed the fluorescent protein mVenus (Nagai et al. 2002) and an in-frame glycine- and serine-rich linker. LOV domains were inserted in this plasmid using PCR (see above). All constructs were verified by DNA sequencing. Cells were transfected with 100 ng expression in each well of 96-well plates (four wells for each construct). Expression was assessed by measuring mVenus fluorescence in a plate reader (BioTek Synergy H1, Bad Friedrichshall, Germany) 16 to 18 h after transfection. Transfection with pcDNA3.1(−) or a FKBP-mVenus fusion protein served as controls. Cytotoxicity measurements were conducted using a tetrazolium dye following the supplier's protocol (EZ4U Cell Proliferation and Cytotoxicity Assay, Biomedica, Vienna, Austria). Absorbance measurements at 450 nm with 620 nm reference were performed in the same plate reader as fluorescence measurements.

### Stimulation and Detection of MAPK Signaling (HEK293 Cells)

Activation of the MAPK pathway was assayed with the PathDetect Elk1 trans-Reporting System (Agilent) consisting of an Elk1 phosphorylation-dependent trans-activator and a luciferase-based trans-reporter. Cells were transfected with 213.3 ng total DNA per well (receptor, trans-activator and trans-reporter at ratio of 1:3:60 or 1:30:600) using Lipofectamine 2000. Six h after transfection medium was replaced with CO₂-independent reduced serum starve medium (Gibco/Life Technologies; supplemented with 0.5% FBS, 2 mM L-Glutamine, 100 U/ml penicilin and 0.1 mg/ml streptomycin) for 18 h at 37° C. Cells were transferred and were either kept under constant illumination for 8 h or were protected from light. Chemical stimulation of imFGFR1 followed the same procedure, except that 10 nM AP20187 ((Clackson 1998); ARIAD Pharmaceuticals, Cambridge, Mass.) were added before transfer to the stimulation incubator. After incubation, plates were washed once with PBS and luciferase was detected with standard, off-the-shelf reagents. These were either Luciferase 1000 Assay System (Promega, Mannheim, Germany) in combination with a microplate reader equipped with an injector (Tecan Infinite 200 Pro, Maennedorf, Switzerland), or ONE-Glo Assay System (Promega) in combination with a microplate reader not equipped with an injector (BioTek Synergy H1). These assay systems provide equivalent results.

### Detection of Additional Signaling Pathways (HEK293 Cells)

Activation of additional mFGFR1-related signaling pathways was assayed with Cignal Reporter assays (Qiagen, Hilden, Germany) consisting of mixtures of inducible pathway focused transcription factor-responsive firefly luciferase constructs and a constitutively expressing Renilla luciferase construct. Cells were transfected with 100.3 ng total DNA per well (receptor and reporter at ratio of 1:300) using Lipofectamine 2000 and thereafter treated as described above for detection of MAPK signaling. Cells were processed with the Dual-Glo® Luciferase Assay System (Promega) and signals detected with the microplate reader.

### Generation of Stable Opto-mFGFR1 Cell Lines and Virus Construction

M38K and SPC212, two malignant pleural mesothelioma cell lines, were maintained in RPMI1640 supplemented with 10% FBS. Telomerase-immortalized microvascular hBE cells were maintained in Clonetics EGM2 MV endothelial growth medium (Lonza, Wakersville, Md.) supplemented with 5% FBS. For retrovirus generation, Opto-mFGFR1 or mCherry as control was subcloned into pQCXIP (Clontech, Mountain View, Calif.) using EcoRI and NotI restriction enzymes. Viral particles were generated in HEK293 cells by co-transfection with the helper plasmids pVSV-G and p-gag-pol-gpt. Supernatants were used to transduce M38K, SPC212 or hBE cells grown to 50% confluency in 6-well plates. Cells were selected with 0.8 μg/ml puromycin for 10 d and transgene expression was verified by immunoblotting.

### Stimulation and Western Blot (M38K, SPC212 and hBE Cells)

For immunoblotting, 5×10⁵ cells were seeded in each well of 6-well plates. After 24 h, medium was replaced with reduced serum medium (M38K, SPC212). After additional 20 h, cells were transferred to the stimulation incubator and illuminated for 1, 5 or 15 min or shielded from light. Cells were then either immediately or after additional 5, 15 or 30 min in the dark washed and lysed in 50 μl lysis buffer per well on ice. Lysates were sonicated and centrifuged (12000 g, 5 min, 4° C.). Fifteen μg protein per lane were separated by SDS-PAGE and electro-blotted onto PVDF membranes. Blots were incubated with primary antibodies (FGFR1, #9740; Erk1/2, #9102; pERK, #9101; PLCγ1 #2822; pPLCγ1 #2821; Akt #9272; pAkt #4058S, Cell Signaling Technology, Danvers, Mass.; dilution 1:1000; FGFRpY653/654, Thermo Scientific, Vienna, Austria, dilution 1:1000; β-actin, Sigma, dilution 1:8000) in blocking solution (3% BSA or 5% skim milk in TBST) overnight at 4° C. Secondary antibodies (HRP-coupled α-rabbit or α-anti mouse IgG, Dako, Glostrup, Denmark) were applied at a dilution of 1:10000 for 2 h at room temperature. Chemiluminescence was developed with WesternC reagent (Biorad, Hercules, Calif.) and signals recorded on X-ray film (GE Healthcare).

### ERK Phosphorylation in Spatially-Confined Illumination Experiments (SPC212 and hBE Cells)

For detection of localized ERK phosphorylation in cell monolayers, SPC212 or hBE cells were grown to confluency in 6-cm petri dishes (SPC212) or 12-well plates (hBE cells). SPC212 were starved in medium without serum for 24 h before illumination. Templates with pinholes of 2 (SPC212) or 5 (hBE cells) mm diameter were used for localized illumination for 5 min. Afterwards cells were washed with cold PBS and fixed with Histofix (Lactan, Graz, Austria) for 10 min. After washing with PBS and permeabilization with Triton X100 (0.25% in PBST) and blocking in 1% BSA in PBST, dishes were incubated with pERK (#9101, Cell Signaling Technology, 1:500 for SPC212, 1:100 for hBE cells) for 1 h. Signal was developed using the UltraVision LP detection system (Thermo Scientific) and 3,30-diaminobenzidine as chromogen. Haematoxylin was used for counterstaining of cell nuclei.

### Live Cell Luminescence in Spatially-Confined Illumination Experiments (HEK293 Cells)

3×10⁶ cells were simultaneously seeded and transfected in a 10 cm dish. Cells were transfected using Lipofectamine 2000 and 24 μg total DNA per dish (receptor, trans-activator and trans-reporter at ratio of 1:3:60). After 16 h, cells were treated and illuminated as described for detection of MAPK signalling. Live cells were processed adding 0.15 mg/ml D-luciferin (PEQlab, Erlangen, Germany) in PBS and then incubated for 10 min at 37° C. Luminescence was detected with a PEQLab Fusion SL imaging system (PEQLab, Erlangen, Germany).

### Cell Proliferation (M38K Cells)

2×10⁴ M38K cells were seeded in each well of 96-well plates. After 24 h, cells were stimulated for 1 h or kept in the dark. FGF2 (Sigma, St. Louis, Mo.) was added as indicated. After 24 h, cells were incubated with 10 μM EdU for 2 h. Subsequently, newly synthesized DNA was stained with Click-iT EdU (Life Technologies) following the manufacturer's protocol and counterstained with 5 μg/ml Hoechst dye. Cells were photographed on a Nikon Ti300 inverted microscope. To determine the percentage of cells with newly synthesized DNA, Hoechst positive nuclei and EdU positive nuclei were counted.

### Cell Cycle (M38K Cells)

Cell cycle distribution was analyzed by flow cytometry. 5×10⁵ M38K cells were seeded in 25 cm² tissue culture flasks. After 24 h, cells were stimulated for 1 h or kept in the dark. After additional 24 h, cells were fixed in ethanol (70%), treated with 50 μg/ml RNAse A and 50 μg/ml propidium iodide (PI). Flow cytometry was performed on a FACSCalibur (BD Biosciences, Schwechat, Austria) and cell cycle distribution calculated with ModFit LT software (Verity Software House, Topsham, Me.).

### Cell Morphology (M38K Cells)

10⁵ M38K cells were seeded in each well of 6-well plates. After 24 h, cells were stimulated for 1 h or kept in the dark. FGF2 (Sigma) or PD166866 (Pfizer Global Research and Development, New London, Conn.) were added as indicated. After additional 24 h, cells were photographed on the Nikon Ti300 microscope. For quantification of cell morphology, all cell perimeters in randomly selected sections of phase contrast images were traced and aspect ratios (defined as length of major axis divided by length of minor axis of a fitted ellipse) calculated with ImageJ software (National Institute of Health). Greater than 50 individual values contributed to each average. Automated analysis yielded comparable results.

### Gene Expression Analysis (M38K Cells)

5×10⁴ M38K cells were seeded into each well of a 6-well plates. After 24 h, cells were illuminated with a cycle of 5 min light/15 min dark for 48 h. Control cells were kept in the dark. Total RNA was extracted with TRIZOL (LifeTechnologies) and reverse transcribed with MMLV reverse transcriptase (Thermo Scientific). cDNAs corresponding to 50 ng RNA per sample were subjected to SYBR green qPCR on an Abi Prism 7500 Sequence Detection System using published primers (cf. Sakuma, 2012; #2508). GAPDH was used for normalization and fold change of expression level was calculated as 2-ddCt of illuminated versus non-illuminated cells.

### Actin Staining (M38K Cells)

5×10⁵ M38K cells were seeded onto coverslips in 6-well plates. After 24 h, cells were illuminated with a cycle of 5 min light/15 min dark for 48 h. Control cells were kept in the dark. Cells were fixed (3.8% formaldehyde), permeabilized (0.5% Triton X100 in PBS) stained with TRITC-phalloidin (1:100, 1% BSA in PBS, overnight at 4° C.) and mounted in Vectashield mounting medium containing DAPI. Micrographs were taken on a Leica fluorescence microscope.

### In Vitro Angiogensis (Sprouting) Assay (hBE Cells)

For spheroid generation, hBE cells were suspended as hanging drops (450 cells in a 25 μl drop) in M199 medium (Sigma) supplemented with 10% FBS, L-glutamine, 2.2 g/I NaHCO₃ and 20% methylcellulose (Sigma) over night in a standard tissue culture incubator. The following day, spheroids were washed in PBS containing 10% FBS, centrifuged, resuspended in Methocel/20% FBS, mixed (1:1) with neutralized rat-tail collagen and seeded into non-adhesive 24-well plates (Greiner Bio-one, Kremsmünster, Austria). After solidification of the collagen, VEGFA (30 ng/ml) or PD166866 (10 μM) were added. Plates were stimulated with light for 5 min every 20 min for 10 h or kept in the dark. After stimulation, 1 ml of 8% paraformaldehyde was added to each well and spheroids were photographed on the Nikon microscope. Cumulative sprout lengths per sphere from at least 8 spheroids per group were measured (ImageJ).

All Optical Evaluation of Pharmacological Compounds (M38K and HEK293 cells)

Test compounds were obtained from the following sources and used at the indicated final concentrations: PD166866 (PD, 5 μM; Pfizer Global Research and Development, Groton Conn.), BIBF1120 (BIBF, 0.5 μM; Nintedanib, Vargatef, Selleck Chemicals, Houston, Tex.), AP24534 (PON, 1 μM; Ponatinib, Selleck Chemicals), AZD6244 (SEL, 0.5 μM; Selumetinib, Selleck Chemicals) UO126 (UO, 10 μM; LC Laboratories, Woburn, Mass.), MK2206 (MK, 10 μM; Selleck Chemicals), LY294002 (LY, 20 μM; LC Laboratories), Imatinib (IMA, 0.5 μM; Selleck Chemicals), Vemurafenib (VEM, 0.5 μM; Selleck Chemicals). Concentrations were adjusted according to published reports and were not cytotoxic the incubation time used.

For M38K cells, cell morphology was evaluated as described above but followed by automated image analysis. Phase contrast images (typically three images from two wells) were automatically analyzed. Images were converted to greyscale, resized and divided into segments for local threshold correction (threshold was defined as most probably intensity multiplied by a constant factor of 0.85; Igor Pro, Wavemetrics, Lake Oswego, Oreg.). Cells were identified and measured in FIJI/ImageJ (Max Planck Society/National Institutes of Health; size limit: 40 to 600 pixel̂2, circularity limit: 0.01 to 1.00). Occasionally, outliers were removed manually. Typically 200 to 1800 individual values contributed to the averages of FIG. 9. For HEK293 cells, MAPK pathway activation was measured using luciferase as described above.

### Example 1

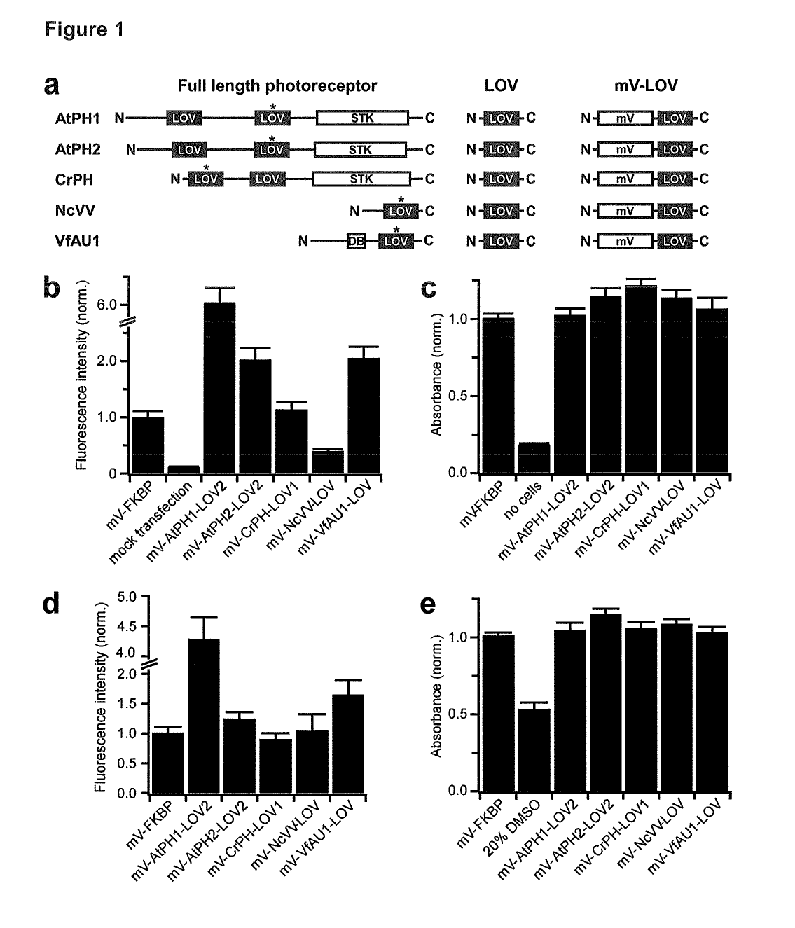

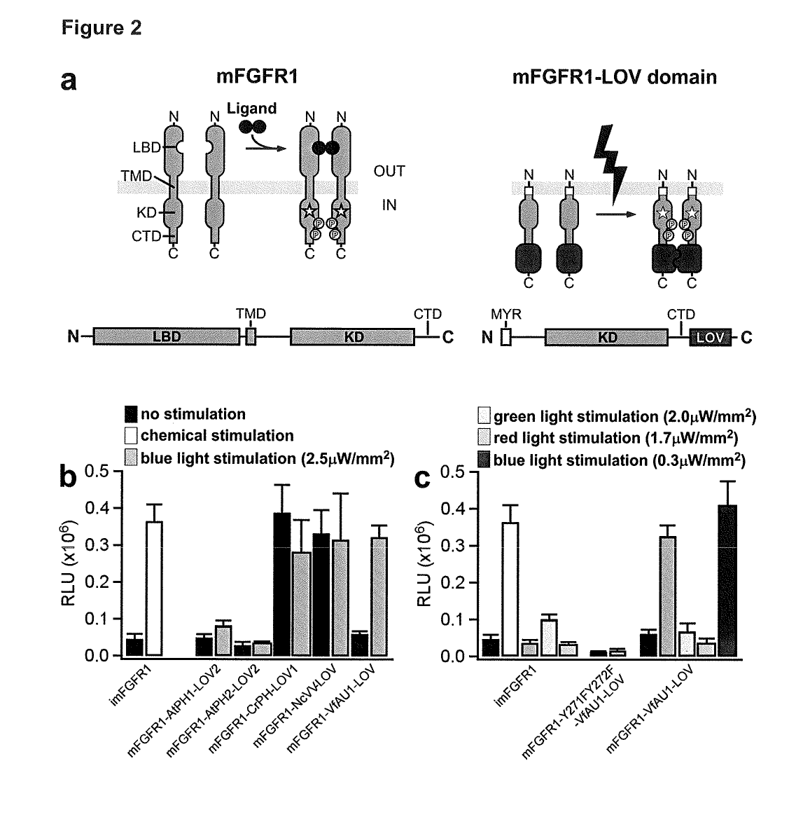

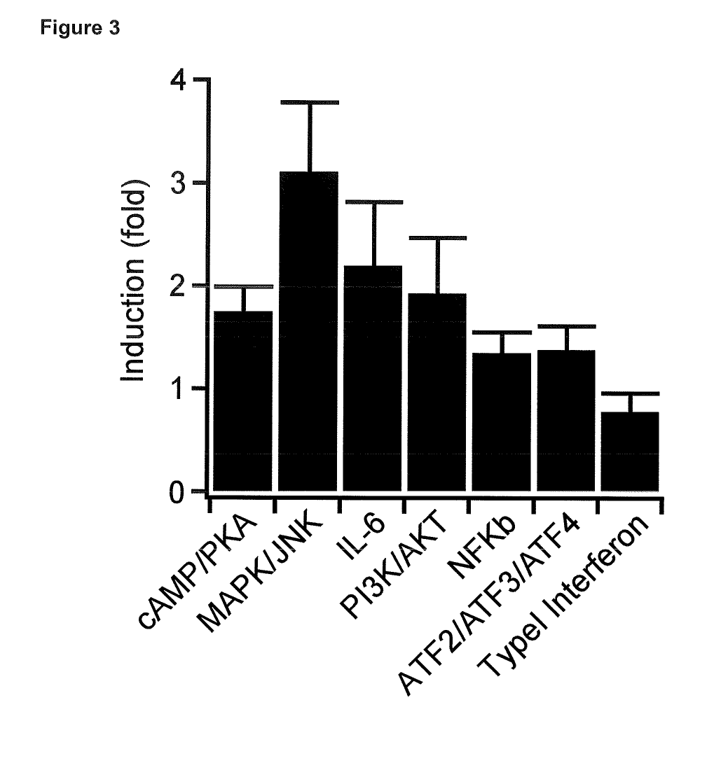

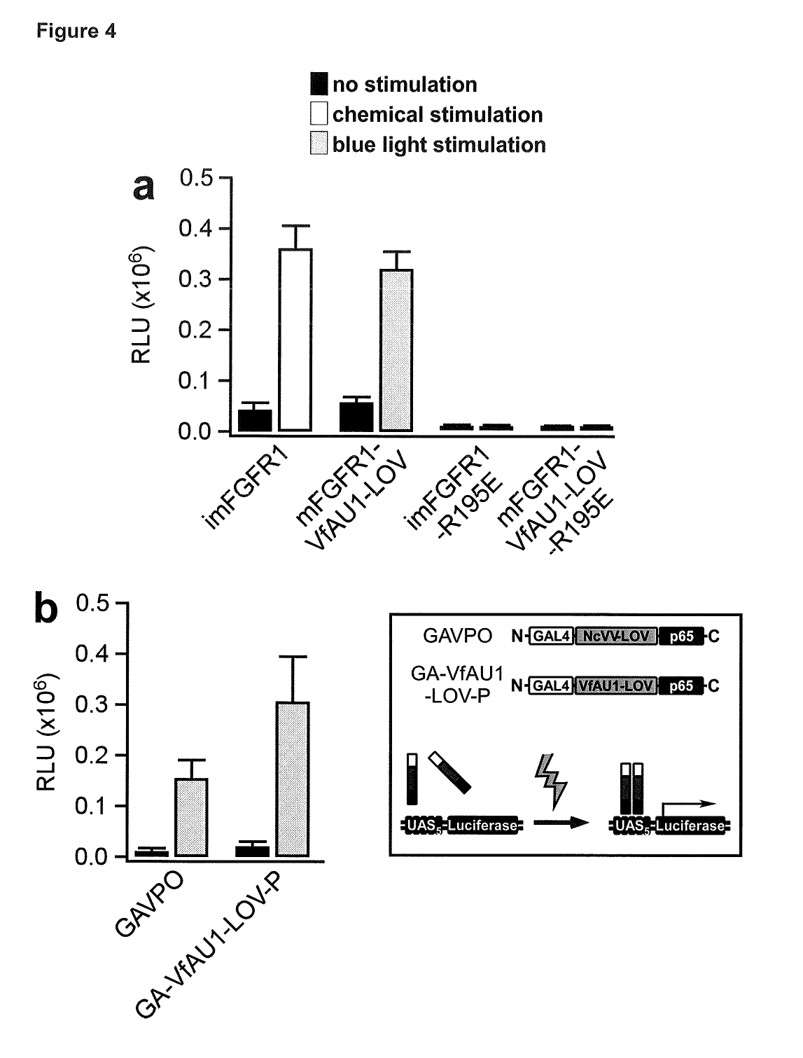

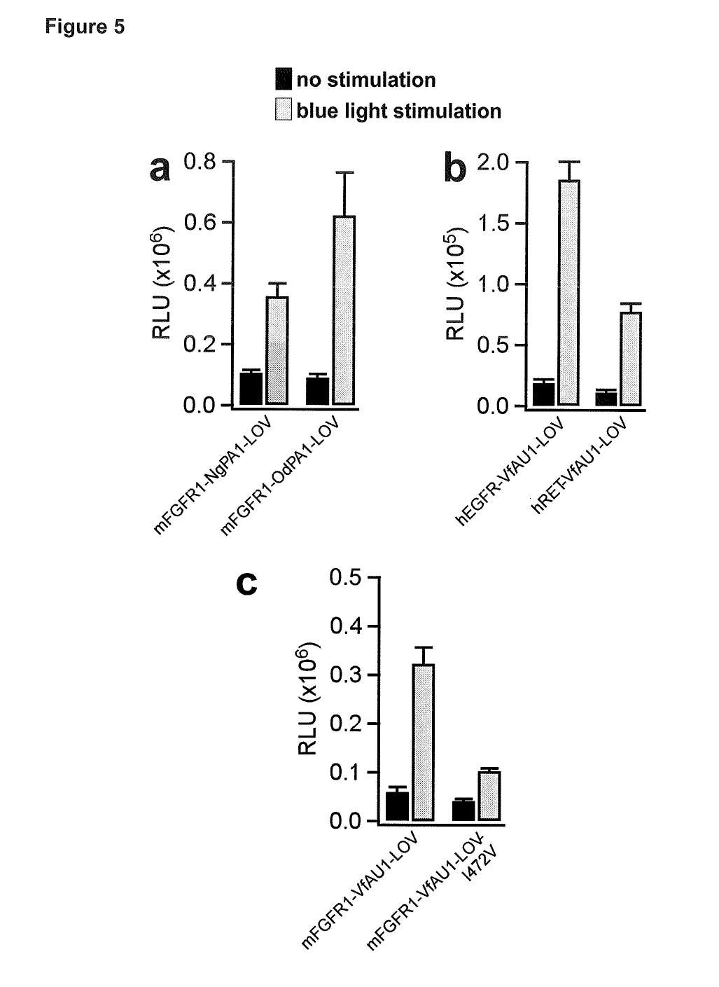

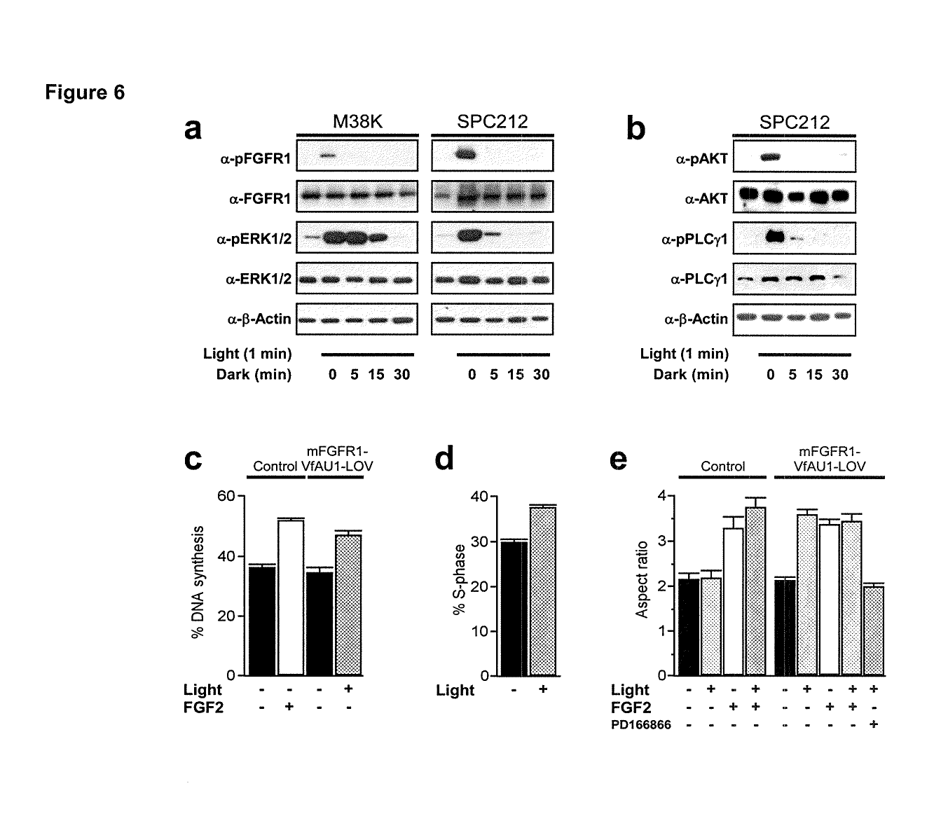

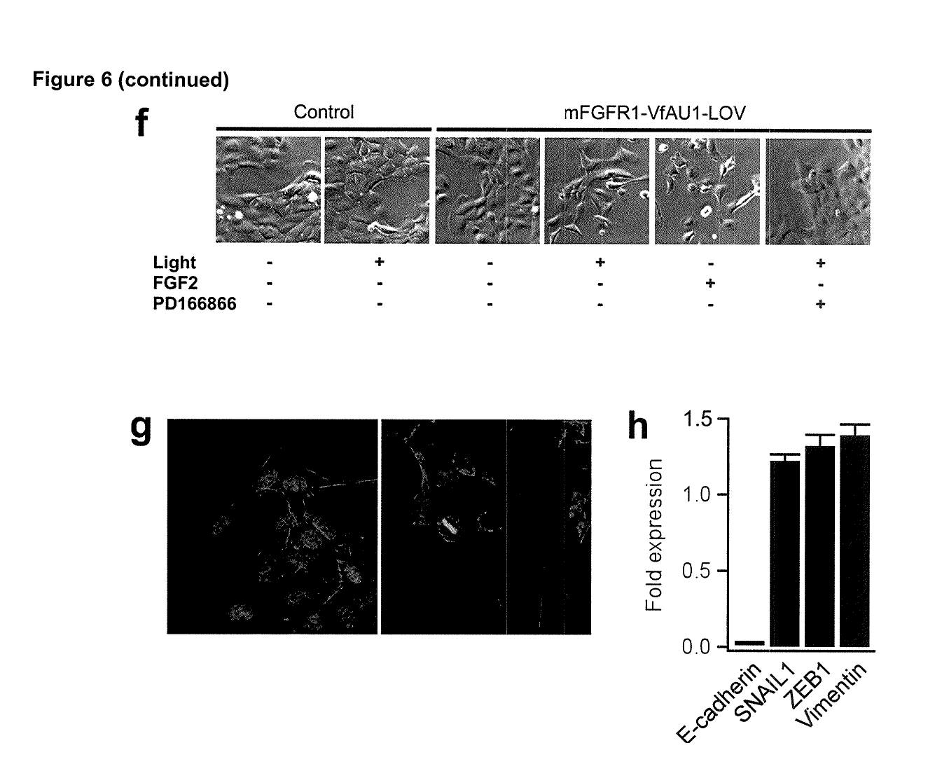

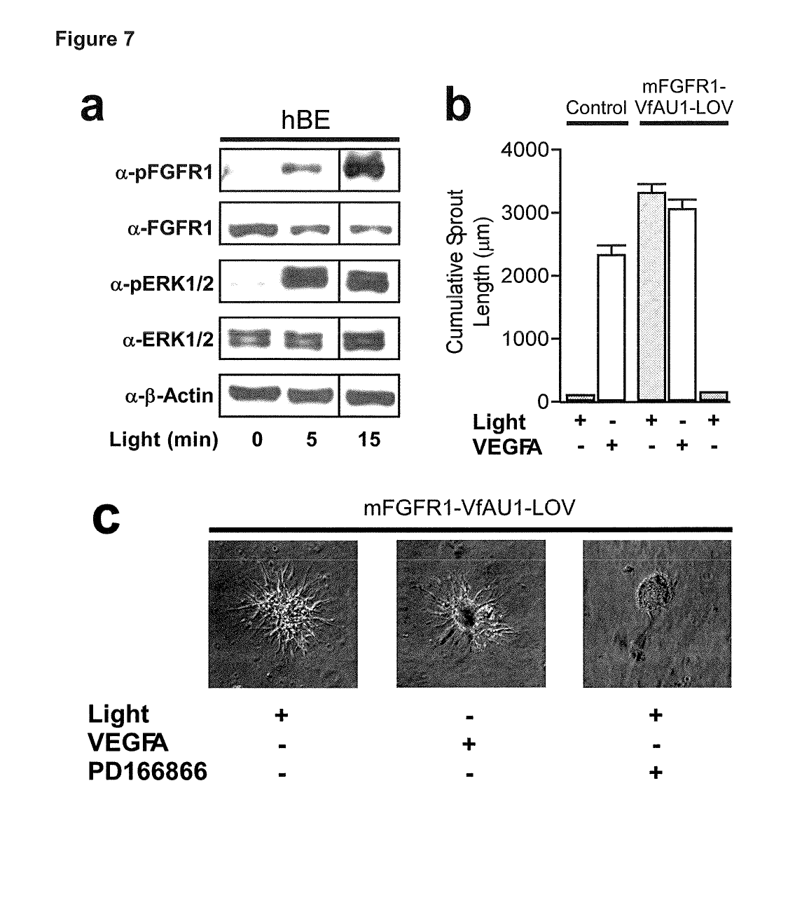

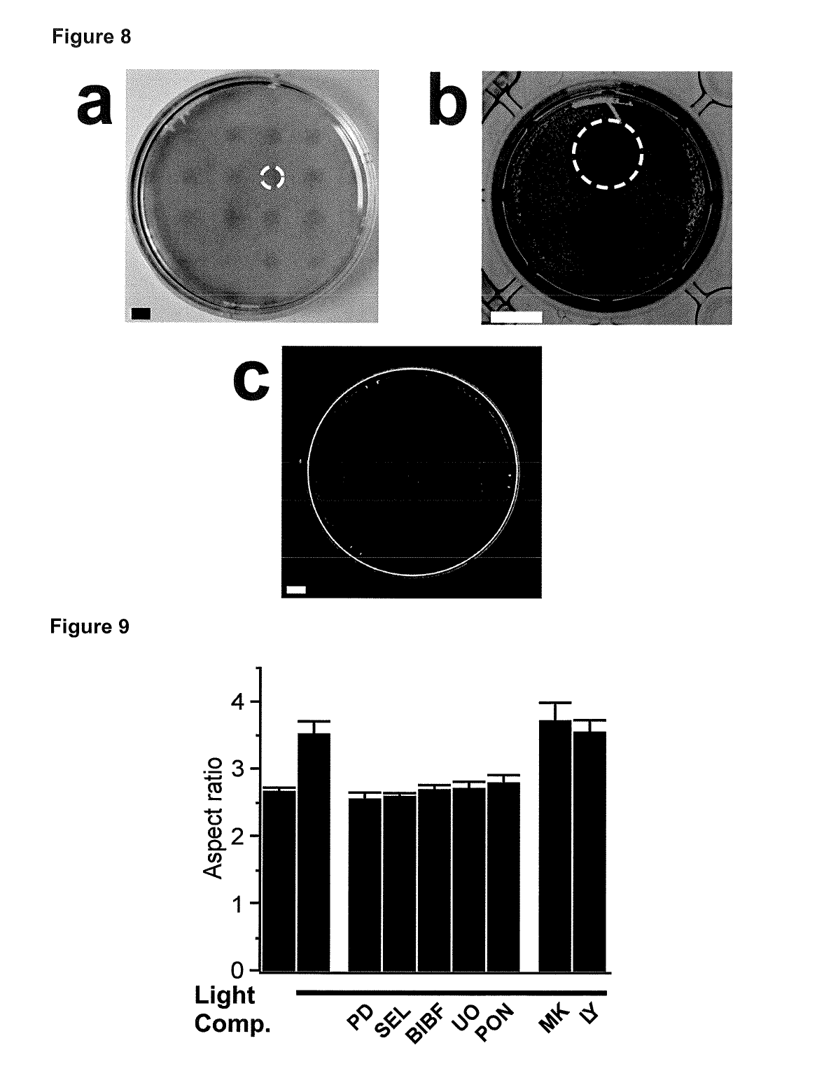

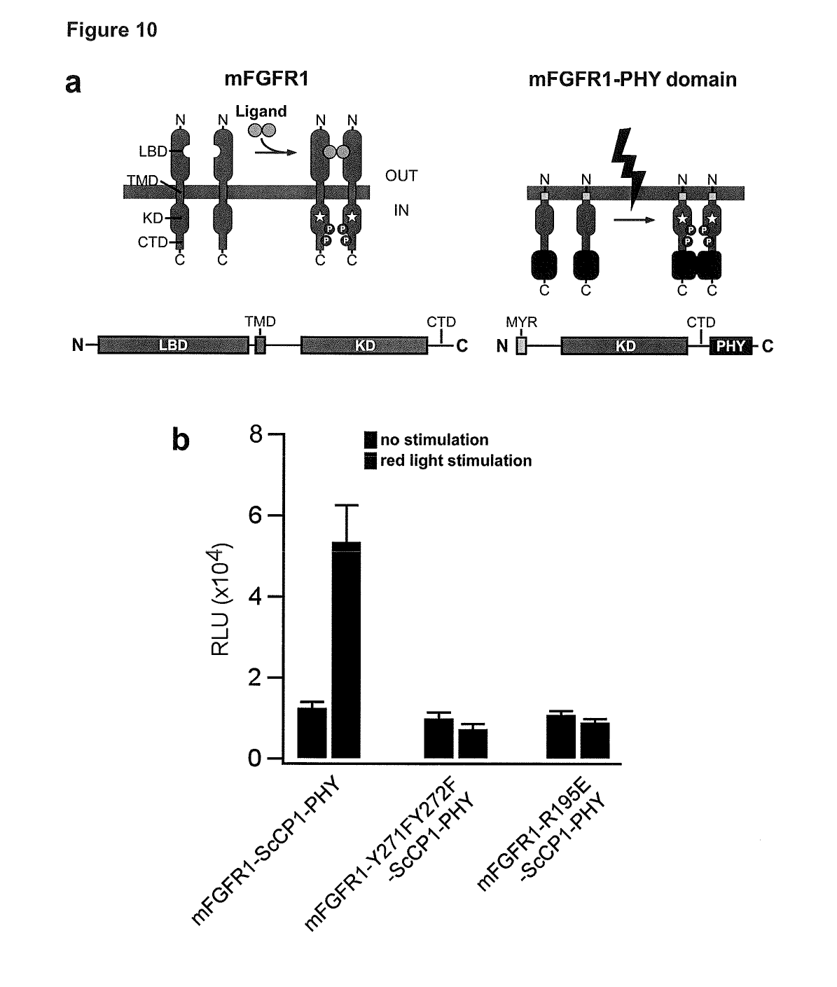

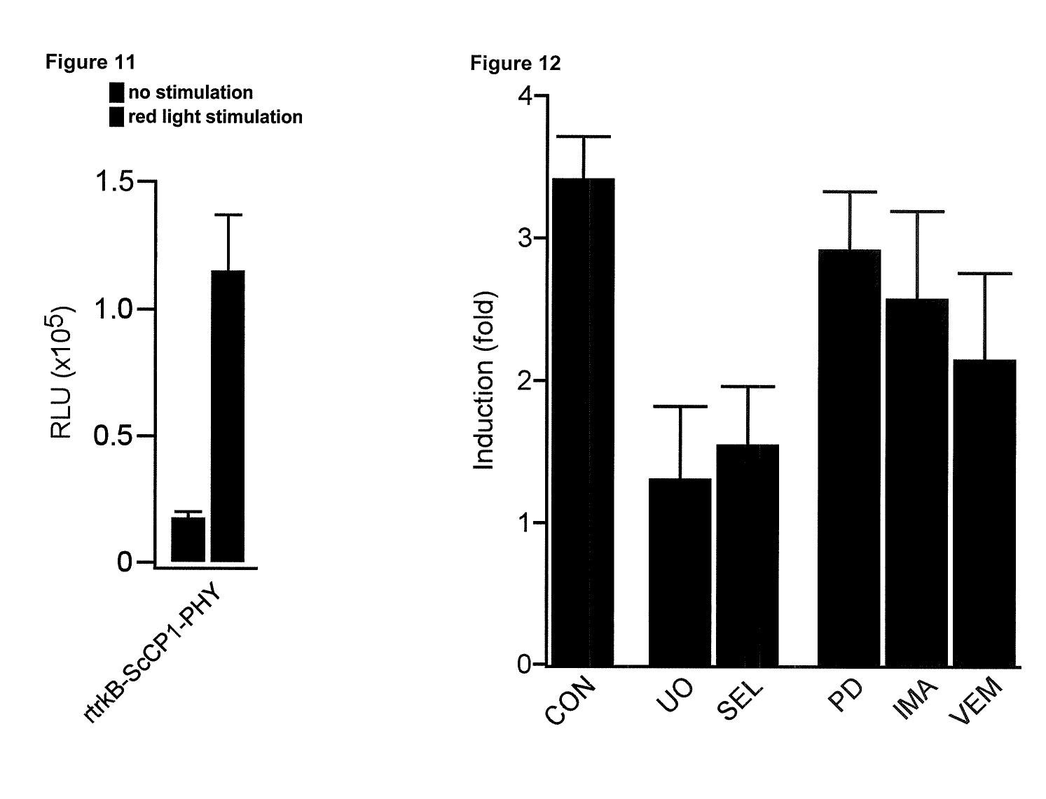

As it was initially unclear which LOV domain will be suited for activation of a mammalian RTK, the inventors compiled an unbiased panel of diverse candidate LOV domains (one from fungi, two from algae and two from plants) (FIG. 1a and SEQ ID NO: 1-14).

TABLE 1
Photophysical and equilibrium binding
parameters of LOV domains.

| | Estimated | Estimated excited |
|---|---|---|
| Name | K_{D} (μM) | state lifetime (s)³ |
| AtPH1-LOV2 | Dark: <25 | ~40 |
| | Light: <25 | |
| AtPH2-LOV2 | Dark: <25 | ~7 |
| | Light: <25 | |
| CrPH-LOV1 | Dark: <55 | ~200¹ |
| | Light: <55 | |
| NcVV-LOV | Dark: <5 | >10'000 |
| | Light: <0.5 | |
| VfAU1-LOV | Dark: >300 | — |
| | Light: <100 | |
| VfAU1-LOV² | — | ~300 |
| VfAU1-LOV² | — | WT: 480 |
| | | I28V (I472V): 60 |

¹A triple exponential decay with lifetimes ranging from 20 to 800 s was observed.
²LOV domains included C- and N-terminal extensions compared to VfAU1-LOV of this study.
³Where necessary, published half life values (t½) were converted to lifetimes (τ = t½/ln(2)) assuming a first order reaction.

For these domains, light-dependent changes in oligomerization state were previously reported (Katsura, 2009; Kaiserli, 2009; Kutta, 2008; Zoltowski, 2008; Toyooka, 2011). As the majority of these domains has never been studied in mammalian cells, the inventors first explored whether these candidate LOV domains can be heterologously expressed from codon-optimized genes in two mammalian cell lines (chinese hamster ovary cells and human embryonic kidney 293 (HEK293) cells, and whether expression causes cytotoxicity. It was found that LOV domains were produced efficiently by both cell lines (as assessed by detection of a fluorescent protein tag), and with no detectable cytotoxicity (as assessed by cellular reduction of a tetrazolium dye) (FIG. 1). Furthermore, no protein aggregates were observed in these cells (data not shown), further supporting proper expression.

The fibroblast growth factor (FGF) receptor 1 (FGFR1) is an evolutionarily conserved RTK and a critical regulator of cellular behavior in embryonic development, adult neurogenesis and tumor formation (Deng et al. 1994, Zhao et al. 2007, Yang et al. 2013). The inventors constructed chimeric receptors where LOV domains are linked to the intracellular domain of murine fibroblast growth factor receptor 1 (mFGFR1). The extracellular ligand-binding modules of mFGFR1 were omitted in the fusion proteins to obtain proteins that are not responsive to native ligands (FIG. 2a). Given the above reasoning, cells expressing fusion proteins should respond to blue light with an activation of signaling pathways characteristic for mFGFR1. The inventors performed cell signaling experiments in a custom-built incubator that allows illuminating mammalian cells with blue light of defined intensity (see Materials and Methods). The inventors first examined the MAPK pathway, a central signaling pathway activated by FGFs via FGFR1 (Ma et al. 2009). As a positive control, the inventors used a modified, chemically-inducible mFGFR1 (imFGFR1; (Welm, Freeman et al. 2002)), which also lacks the ligand-binding modules and is activated by binding of the small chemical ligand AP20187 to a single, engineered FK506 binding domain (FKBP). These experiments revealed that the fusion protein incorporating the LOV domain of aureochrome1 from V. frigida (VfAU1-LOV-mFGFR1) activated the MAPK pathway similarly to imFGFR1. In particular, no augmented basal pathway activation in the absence of light was observed and pathway induction by light was of comparable magnitude to pathways activation by ligand (FIG. 2b). All other fusion proteins either exhibited no activity or constitutive activity (FIG. 2b). Control experiments showed that (i) blue light had no effect on cells that express imFGFR1, (ii) blue light had no effect on cells that express VfAU1-LOV-mFGFR1 after loss of kinase activity, and (iii) green light or red light had no effect on cells that express VfAU1-LOV-mFGFR1 (FIG. 2c). Collectively, these results indicate that VfAU1-LOV-mFGFR1, a chimeric receptor consisting of the catalytic domain of a mammalian RTK and an algal LOV domain, activates the canonical MAPK signaling pathway (FIG. 2) and additional pathways linked to mFGFR1 (FIG. 3) in response to blue light. The inventors termed this receptor “Opto-mFGFR1”

The inventors next investigated whether dimerization is underlying the activation of Opto-mFGFR1. A single charge inversion mutation (R557E in full length FGFR1; R195E in Opto-FGFR1 or miFGFR1) prevents formation of a functionally essential, asymmetric kinase domain dimer in FGFR1 (Bae et al. 2010) and inhibits MAPK activation by imFGFR1 (FIG. 4a). The inventors introduced this mutation into Opto-mFGFR1 as a probe for dimer formation during activation. In cells expressing Opto-mFGFR1-R195E, no MAPK pathway activation in response to blue light was detectable (FIG. 4a), indicating that receptor dimerization is required for receptor activation. This result, together with the observation that VfAU1-LOV dimerizes in response to blue light in mammalian cells (FIG. 4b), points to dimerization as the mechanism underlying of Opto-mFGFR1 activation.

The inventors further tested whether LOV domains that resemble VfAU1-LOV can activate mFGFR1. Using database searches, the inventors identified VfAU1-like proteins in the eustigmatophyte Nannochloropsis gaditana (N. gaditana hypothetical protein (NgPA1)) and in the golden algae Ochromonas danica (O. danica putative aureochrome1 (OdPA1)). For mFGFR1 fusion proteins incorporating LOV domains of NgPA1 and OdPA1 (NgPA1-LOV and OdPA1-LOV), the inventors also observed blue light-induced activation of MAPK signaling with amplitudes similar to that of the original Opto-mFGFR1 (FIG. 5a). Thus LOV domains of multiple aureochrome-like proteins are capable of mFGFR1 activation.

To test whether VfAU1-LOV is capable of activating other RTKs, the inventors combined it with the catalytic domain of the human epidermal growth factor receptor (hEGFR) and human RET (hRET). The inventors followed the design established in Opto-mFGFR1. In line with their known signalling capability, robust activation of the MAPK pathway by light was observed in cells expressing the hEGFR and hRET fusion proteins (FIG. 5b). These fusion proteins were termed “Opto-hEGFR” and “Opto-hRET”.

In the rational design of the first light-activated RTK, the inventors replaced ligand-induced dimerization by a light-activated protein-protein interaction. Because of the absence of precedence for light-controlled mammalian receptor dimerization, and because of the structural diversity of naturally-occurring photoreceptors (Moglich et al. 2010, Zoltowski and Gardner 2011), the inventors initially followed an unbiased approach and evaluated five LOV domains originating from four different non-animal species. The successful identification of VfAU1-LOV supports the notion that Nature offers a large repertoire of light-sensitive molecular functionalities that can be harvested in light-activated molecular tools (Chow et al. 2010). Incorporation of VfAU1-LOV endows Opto-mFGFR1 with several beneficial features. As the photosensory element, VfAU1-LOV incorporates flavin mononucleotide (FMN), a prosthetic group of oxidoreductases that are abundantly present in most if not all animal cells. Opto-mFGFR1 thus is expected to function in many cell types without the need for addition of an exogenous co-factor, a critical feature for optogenetic experiments in vivo, and the inventors demonstrated function in three cell types that were not supplemented with FMN. Second, Opto-mFGFR1 is efficiently activated by low intensity blue light (e.g. ˜3 μW/mm², FIG. 2), which is readily achieved in transparent animal models and transdermally in rodents (Janovjak et al. 2010, Ye et al. 2011).

A comparison of the five LOV domains initially evaluated allows proposing and experimentally validating those properties of VfAU1-LOV that contribute to its function in Opto-mFGFR1. First, only in VfAU1 but not in the other four photoreceptors is the LOV domain located C-terminal to the effector domain (FIG. 1). Furthermore, previously uncharacterized LOV domains that are also located C-terminal of effector domains in their full length photoreceptors can functionally replace VfAU1-LOV (OdPA1-LOV and NgPA1-LOV; FIG. 5a). Thus, preserving the domain order of the naturally-occurring photoreceptor appears to be beneficial for the function of the engineered protein. In turn, the inventors propose that the full length receptors OdPA1 and NgPA1 function by a similar mechanism as VfAU1, and thus corroborate the view that engineering of light-activated proteins may allow insights into the function and discovery of naturally-occurring proteins (Janovjak, Szobota et al. 2010, Janovjak et al. 2011).

Second, while dark state dimerization was observed for most if not all characterized LOV domains, it occurs for VfAU1-LOV at concentrations that are one to two orders of magnitude higher than for other domains (Table 1 above). Thus, incorporating a domain with little or no dark state dimerization at concentrations <100 μM appears to be beneficial for the function of the engineered protein.

Third, it is reasonable to assume that the photo-excited state of VfAU1-LOV must be sufficiently long-lived to allow for receptor dimerization and stabilization of receptor dimers. Ligands establish functional FGFR1 dimers for 30 to 100 s (Powell et al. 2002), and these values are shorter than the photo-excited state lifetime of VfAU1-LOV but not of some of the other domains (Table 1 above). In line with this model, reducing the lifetime of VfAU1-LOV ˜8-fold by mutation reduces Opto-mFGFR1 activation (FIG. 5c). A combination of the above mentioned properties, domain order, dark dimerization and photo-excited state lifetime, appears required for mFGFR1 activation by a LOV domain.

Collectively, these results indicate that fusion proteins consisting of LOV domains (NgPA1-LOV, OdPA1-LOV, and VfAU1-LOV) and the catalytic domain of mammalian RTKs (mFGFR1, hEGFR, and hRET) activates the cell signaling pathways linked to RTKs in response to blue light.

### Example 2

The development and progression of cancer is frequently linked to mutations in RTKs or RTK overexpression, and many cancer cells respond to growth factors with increased proliferation, migration and epithelial-mesenchymal transition (EMT) (Metzner et al. 2011, Sakuma et al. 2012). To establish a cellular model of human cancer relevant to FGF/FGFR signaling, the inventors tested cells from different tumor entities for effects of FGF2, a prominent FGFR ligand. The inventors found that M38K cells (Kahlos et al. 1998) derived from malignant pleural mesothelioma responded to FGF with characteristic changes in cell behavior. To investigate whether Opto-FGFR1 allows controlling the behavior of these human tumor cells with light, the inventors virally delivered Opto-mFGFR1 into these cells and propagated cells with stable Opto-mFGFR1 expression. Stimulation with blue light resulted in rapid phosphorylation of Opto-mFGFR1 and ERK1/2, which returned to pre-stimulation levels within minutes after cessation of light (FIG. 6a). Likewise, rapid phosphorylation of Opto-mFGFR1 and ERK1/2 as well as AKT and phospholipase Cy (PLCy), additional signaling molecules regulated by FGF (Ma, Ponnusamy et al. 2009, Coutu et al. 2011), was observed in a second FGF2-responsive cell line derived from malignant pleural mesothelioma (SPC212; (Schmitter et al. 1992)) (FIGS. 6a and b). Moreover, light stimulation triggered increased proliferation (assessed as % of nuclei incorporating 5-ethynyl-2′-deoxyuridine), a shift of cell cycle distribution towards the S-phase, EMT-like morphological alterations and EMT-like gene expression changes comparable to those of FGF2-treated cells (FIG. 6c to g). Light-induced changes in morphology were inhibited by pre-treatment with PD166866, a selective FGFR1 inhibitor (FIGS. 6e and f). In addition, in blood endothelial cells, a model system not related to cancer, stimulation with blue light also resulted in rapid phosphorylation of Opto-mFGFR1 and ERK1/2 (FIG. 7a). Also in this system, blue light illumination induced morphological alterations (FIGS. 7b and c).

For Opto-mFGFR1, temporally restricted optical stimulation demonstrated the ability to control receptor activation on time scales that are comparable to other widely used optogenetic tools (Kennedy et al. 2010) and more rapid than those relevant in physiology and development (FIGS. 6a and b, FIG. 7a), while spatially restricted optical stimulation demonstrates the ability of localized receptor activation (FIG. 8).

It was recently proposed that light-activated proteins may enable novel approaches for the evaluation of pharmacological agents (Prigge, Rosier et al. 2010, Entcheva 2013). These ideas build on using light both as the activator and read-out of cellular signals and thereby allow for simplification and cost reduction. Proof-of-concept for this approach with multiple molecules is currently not available. The inventors experimentally realized an “all optical” evaluation of small molecules based on the optical activation of disease related cellular signaling by Opto-mFGFR1 and based on morphology changes of M38K cells. The inventors focused on inhibitors for FGFR1 and other kinases with the expectation that inhibitors specific for FGFR1, and in turn also the downstream pathway responsible for morphology changes, can be identified using M38K cells as a model system. The inventors found that morphology changes could be abrogated by treatment with the FGFR inhibitors PD166866, BIBF1120 and Ponatinib as well as with the MEK inhibitors UO126 and Selumetinib. The PI3K inhibitor LY294002 and the Akt inhibitor MK2206 were not effective.

These results demonstrate (i) that in M38K cells the morphology changes depend on the MAPK pathway, whereas signals from the PI3K/Akt pathwayare dispensible, and (ii) an all optical pharmacological evaluation to identify inhibitors interfering with activation of specific receptors and pathways.

In contrast to cells of the nervous system, for which optogenetic tools are valuable established drivers of cellular activity and neural circuits, optical control of the behavior of cancer cells has not been realized to date. The inventors employ Opto-mFGFR1 to regulate behaviors characteristic for malignant cells, such as cell proliferation, cell morphology and cell migration in cellular models of malignant pleural mesothelioma. The activation of a single component, Opto-mFGFR1, is sufficient to produce these behavioral changes. As RTKs are key players in development and cell fate decisions, and the inventors expect that light-activated RTKs enable novel investigations of these processes, for instance in spatial and temporal activation patterns. FGFR1 specifically has been shown to control self-renewal and differentiation of mesenchymal and neuronal stem cells via distinct pathways (Ma, Ponnusamy et al. 2009, Coutu, Francois et al. 2011), all of which can be controlled by Opto-mFGFR1.

Despite a large potential, applications of optogenetics in biotechnology are rare. For ion channels, it was proposed that the non-invasive nature of optical control may be taken advantage of in a more rapid and contactless evaluation of molecules that affect membrane currents (Prigge, Rosier et al. 2010, Entcheva 2013). The inventors established an “all optical” screening method based on Opto-mFGFR1 and in a high-throughput compatible format. This method employs light both as a stimulus and as readout for activation and detection of cellular signaling. In these validation experiments, compounds that are inhibitors of FGFR1 as well as inhibitors of downstream targets could be identified and the pathway underlying changes in M38K cell behavior could be identified. The design of this experiment matches pharmacological scenarios that aim at inhibition of signaling pathways rather and inhibition of predefined components of pathways as some components might be easier to target or have higher specificity than others. Replacement of chemical activators by light may yield operational simplification and cost reduction while maintaining temporal control of activation and tuning of activation strength. Furthermore, optical activation of engineered receptors is specific for the incorporated receptor-type and avoids potential complications caused by the absence of subtype-specific ligands for receptor families or subtypes. However, the possibility of parallelization is maintained as a large variety of receptors/signaling pathways are activated using light as a single, universal input.

### Example 3

The inventors first identified a protein domain that undergoes homodimerization in response to red light (the light-sensing domain of the cyanobacterial phytochrome (PHY) CPH1 of Synechocystis PCC6803 (SyCP1-PHY)). The inventors then prepared fusion proteins where SyCP1-PHY was linked to the intracellular catalytic domain of murine FGFR1 (mFGFR1) or rat trkB (rtrkB) (FIGS. 10 and 11). The extracellular ligand-binding modules of mFGFR1/rtrkB were omitted to obtain fusion proteins that are not responsive to native ligands. Cells expressing the fusion proteins should respond to red light with activation of signalling pathways characteristic for mFGFR1/rtrkB. The inventors performed cell signalling experiments in a custom-built incubator that allows illumination of cells and tissues with light of defined intensity and colour (Materials and Methods). As in Example 1, the mitogen-activated protein kinase (MAPK) pathway was first examined.

It was found that the fusion proteins activated the MAPK pathway in response to low intensity red light illumination (FIGS. 10 and 11). Control experiments showed that red light had no effect on cells transfected with kinase-dead mFGFR1-SyCP1-PHY or mFGFR1-SyCP1-PHY that is dimerization incompetent (FIG. 10). The results demonstrate that mFGFR1-SyCP1-PHY and rtrkB-SyCP1-PHY, chimeric receptors consisting of the catalytic domain of a mammalian RTK and a cyanobacterial PHY domain, activate the canonical MAPK pathway in response to red light. The inventors termed these receptors “redOpto-mFGFR1” and “redOpto-rtrkB”.

### Example 4

It was recently proposed that light-activated proteins may enable novel approaches for the evaluation of pharmacological agents (Prigge, Rosier et al. 2010, Entcheva 2013). These ideas build on using light both as the activator and read-out of cellular signals and thereby allow for simplification and cost reduction. Proof-of-concept for this approach with multiple molecules is currently not available. The inventors experimentally realized an “all optical” evaluation of small molecules based on the optical activation of disease related cellular signaling by redOpto-rtrkB and based on luciferase signals of HEK293 cells. The inventors focused on inhibitors of the MAPK pathway and other kinases with the expectation that inhibitors specific for the MAPK pathway can be identified (FIG. 12). The inventors found that MAPK pathway induction in response to red light could be abrogated by treatment with the MEK inhibitors UO126 and Selumetinib. The FGFR inhibitor PD166866, the CKIT inhibitor Imatinib and the BRAF inhibitor Vemurafenib were not effective.

These results further demonstrate that an all optical pharmacological evaluation to identify inhibitors interfering with activation of specific receptors and pathways.

redOpto-mFGFR1 and redOpto-rtrkB exemplify a highly valuable class of optogenetic tools, since red light offers markedly improved tissue penetration compared to blue light. For instance, bone/skull of 5 mm thickness transmits ˜2% of blue (460 nm) but ˜10% of red (640 nm) light (Wan, Parrish et al. 1981). Or, muscle tissue of 1 cm thickness transmits ˜20% of blue but ˜80% of red light (Marquez, Wang et al. 1998).

In addition, the combination of PHY- and LOV domain-containing receptor families enables experiments with dual-color activation.

### LIST OF REFERENCES

WO 2013/133643
WO 2013/074911
WO 2013/003557
WO 2012/116621
WO 2010/006049
WO 2009/151948
WO 2009/148946
WO 2008/089003
WO 2008/086470
WO 2007/024391
WO 1999/036553
US 2013/0116165
US 2010/0234273
US 2009/0233364
US 2006/0110827
Bae, J. H., T. J. Boggon, F. Tome, V. Mandiyan, I. Lax and J. Schlessinger (2010). “Asymmetric receptor contact is required for tyrosine autophosphorylation of fibroblast growth factor receptor in living cells.” Proc Natl Acad Sci USA 107(7): 2866-2871.
Burke, C. L. and D. F. Stern (1998). “Activation of Neu (ErbB-2) mediated by disulfide bond-induced dimerization reveals a receptor tyrosine kinase dimer interface.” Mol Cell Biol 18(9): 5371-5379.
Casaletto, J. B. and A. I. McClatchey (2012). “Spatial regulation of receptor tyrosine kinases in development and cancer.” Nat Rev Cancer 12(6): 387-400.
Chow, B. Y. and E. S. Boyden (2013). “Optogenetics and translational medicine.” Sci Transl Med 5(177): 177ps175.
Chow, B. Y., X. Han, A. S. Dobry, X. Qian, A. S. Chuong, M. Li, M. A. Henninger, G. M. Belfort, Y. Lin, P. E. Monahan and E. S. Boyden (2010). “High-performance genetically targetable optical neural silencing by light-driven proton pumps.” Nature 463(7277): 98-102.
Clackson, T. (1998). “Redesigning small molecule-protein interfaces.” Curr Opin Struct Biol 8(4): 451-458.
Cochran, J. R., D. Aivazian, T. O. Cameron and L. J. Stern (2001). “Receptor clustering and transmembrane signaling in T cells.” Trends Biochem Sci 26(5): 304-310.
Coutu, D. L., M. Francois and J. Galipeau (2011). “Inhibition of cellular senescence by developmentally regulated FGF receptors in mesenchymal stem cells.” Blood 117(25): 6801-6812.
Deng, C. X., A. Wynshaw-Boris, M. M. Shen, C. Daugherty, D. M. Ornitz and P. Leder (1994). “Murine FGFR-1 is required for early postimplantation growth and axial organization.” Genes Dev 8(24): 3045-3057.
Entcheva, E. (2013). “Cardiac optogenetics.” Am J Physiol Heart Circ Physiol 304(9): H1179-1191.
Fenno, L., O. Yizhar and K. Deisseroth (2011). “The development and application of optogenetics.” Annu Rev Neurosci 34: 389-412.
Heintzen, C., J. J. Loros and J. C. Dunlap (2001). “The PAS protein VIVID defines a clock-associated feedback loop that represses light input, modulates gating, and regulates clock resetting.” Cell 104(3): 453-464.
Huang, K., T. Merkle and C. F. Beck (2002). “Isolation and characterization of a Chlamydomonas gene that encodes a putative blue-light photoreceptor of the phototropin family.” Physiol Plant 115(4): 613-622.
Huang, Y., L. Wang, M. Zheng, L. Zheng, Y. Tong and Y. Li (2013). “Overexpression of NgAUREO1, the gene coding for aureochrome 1 from Nannochloropsis gaditana, into Saccharomyces cerevisiae leads to a 1.6-fold increase in lipid accumulation.” Biotechnol Lett. DOI 10.10071s10529-013-1386-0.
Janovjak, H., G. Sandoz and E. Y. Isacoff (2011). “A modern ionotropic glutamate receptor with a K(+) selectivity signature sequence.” Nat Commun 2: 232.
Janovjak, H., S. Szobota, C. Wyart, D. Trauner and E. Y. Isacoff (2010). “A light-gated, potassiumselective glutamate receptor for the optical inhibition of neuronal firing.” Nat Neurosci 13(8): 1027-1032.
Kahlos, K., S. Anttila, T. Asikainen, K. Kinnula, K. O. Raivio, K. Mattson, K. Linnainmaa and V. L. Kinnula (1998). “Manganese superoxide dismutase in healthy human pleural mesothelium and in malignant pleural mesothelioma.” Am J Respir Cell Mol Biol 18(4): 570-580.
Kennedy, M. J., R. M. Hughes, L. A. Peteya, J. W. Schwartz, M. D. Ehlers and C. L. Tucker (2010). “Rapid blue-light-mediated induction of protein interactions in living cells.” Nat Methods 7(12): 973-975.
Kim, N., Kim J. M., Lee, M. Kim, C. Y., Chang, K. and Heo, W. D. (2014) “Spatiotemporal Control of Fibroblast Growth Factor Receptor Signals by Blue Light.” Chem Biol. 21: 903-912.
Kinoshita, T., M. Doi, N. Suetsugu, T. Kagawa, M. Wada and K. Shimazaki (2001). “Phot1 and phot2 mediate blue light regulation of stomatal opening.” Nature 414(6864): 656-660.
Lemmon, M. A. and J. Schlessinger (2010). “Cell signaling by receptor tyrosine kinases.” Cell 141(7): 1117-1134.
Ma, D. K., K. Ponnusamy, M. R. Song, G. L. Ming and H. Song (2009). “Molecular genetic analysis of FGFR1 signalling reveals distinct roles of MAPK and PLCgamma1 activation for self-renewal of adult neural stem cells.” Mol Brain 2: 16.
Marquez, G., L. V. Wang, S. Lin, J. A. Schwartz and S. L. Thomsen (1998). “Anisotropy in the absorption and scattering spectra of chicken breast tissue.” Appl Opt 37(4): 798-804.
McCarty, M. F. (2007). “Clinical potential of Spirulina as a source of phycocyanobilin.” J Med Food 10(4): 566-570.
Metzner, T., A. Bedeir, G. Held, B. Peter-Vorosmarty, S. Ghassemi, C. Heinzle, S. Spiegl-Kreinecker, B. Marian, K. Holzmann, B. Grasl-Kraupp, C. Pirker, M. Micksche, W. Berger, P. Heffeter and M. Grusch (2011). “Fibroblast growth factor receptors as therapeutic targets in human melanoma: synergism with BRAF inhibition.” J Invest Dermatol 131(10): 2087-2095.
Moglich, A., X. Yang, R. A. Ayers and K. Moffat (2010). “Structure and function of plant photoreceptors.” Annu Rev Plant Biol 61: 21-47.
Möglich et al. (2010) Photochem. Photobiol. Sci. 9: 1286-1300.
Müller & Weber (2013) Mol. Biosyst. 9: 596-608.
Muthuswamy, S. K., M. Gilman and J. S. Brugge (1999). “Controlled dimerization of ErbB receptors provides evidence for differential signaling by homo- and heterodimers.” Mol Cell Biol 19(10): 6845-6857.
Nagai, T., K. Ibata, E. S. Park, M. Kubota, K. Mikoshiba and A. Miyawaki (2002). “A variant of yellow fluorescent protein with fast and efficient maturation for cell-biological applications.” Nat Biotechnol 20(1): 87-90.
Pathak et al. (2013) Biol. Cell 105: 59-72.
Powell, A. K., D. G. Fernig and J. E. Turnbull (2002). “Fibroblast growth factor receptors 1 and 2 interact differently with heparin/heparan sulfate. Implications for dynamic assembly of a ternary signaling complex.” J Biol Chem 277(32): 28554-28563.
Prigge, M., A. Rosier and P. Hegemann (2010). “Fast, repetitive light-activation of CaV3.2 using channelrhodopsin 2.” Channels (Austin) 4(3): 241-247.
Robertson, S. C., J. A. Tynan and D. J. Donoghue (2000). “RTK mutations and human syndromes when good receptors turn bad.” Trends Genet 16(6): 265-271.
Sakuma, K., M. Aoki and R. Kannagi (2012). “Transcription factors c-Myc and CDX2 mediate E-selectin ligand expression in colon cancer cells undergoing EGF/bFGF-induced epithelial-mesenchymal transition.” Proc Natl Acad Sci USA 109(20): 7776-7781.
Schmidt et al. (August 2013) Nature Communications, DOI: 10.1038/ncomms4019.
Schmitter, D., B. Lauber, B. Fagg and R. A. Stahel (1992). “Hematopoietic growth factors secreted by seven human pleural mesothelioma cell lines: interleukin-6 production as a common feature.” Int J Cancer 51(2): 296-301.
Shilo, B. Z. (2005). “Regulating the dynamics of EGF receptor signaling in space and time.” Development 132(18): 4017-4027.
Simi, A. and C. F. Ibanez (2010). “Assembly and activation of neurotrophic factor receptor complexes.” Dev Neurobiol 70(5): 323-331.
Spaargaren, M., L. H. Defize, J. Boonstra and S. W. de Laat (1991). “Antibody-induced dimerization activates the epidermal growth factor receptor tyrosine kinase.” J Biol Chem 266(3): 1733-1739.
Strauss, H. M., P. Schmieder and J. Hughes (2005). “Light-dependent dimerization in the N-terminal sensory module of cyanobacterial phytochrome 1.” FEBS Lett 579(19): 3970-3974.
Szobota, S. and E. Y. Isacoff (2010). “Optical control of neuronal activity.” Annu Rev Biophys 39: 329-348.
Takahashi, F., D. Yamagata, M. Ishikawa, Y. Fukamatsu, Y. Ogura, M. Kasahara, T. Kiyosue, M. Kikuyama, M. Wada and H. Kataoka (2007). “AUREOCHROME, a photoreceptor required for photomorphogenesis in stramenopiles.” Proc Natl Acad Sci USA 104(49): 19625-19630.
Toyooka, T., O. Hisatomi, F. Takahashi, H. Kataoka and M. Terazima (2011). “Photoreactions of aureochrome-1.” Biophysical J 100(11): 2801-2809.
Tucker, C. L. (2012). “Manipulating cellular processes using optical control of protein-protein interactions.” Prog Brain Res 196: 95-117.
Wan, S., J. A. Parish, R. R. Anderson and M. Madden (1981). “Transmittance of nonionizing radiation in human tissues.” Photochem Photobiol B 34: 679 to 681.
Wang, X., X. Chen and Y. Yang (2012). “Spatiotemporal control of gene expression by a light switchable transgene system.” Nat Methods 9(3): 266-269.
Welm, B. E., K. W. Freeman, M. Chen, A. Contreras, D. M. Spencer and J. M. Rosen (2002). “Inducible dimerization of FGFR1: development of a mouse model to analyze progressive transformation of the mammary gland.” J Cell Biol 157(4): 703-714.
Wend, S., H. J. Wagner, K. Müller, M. D. Zurbriggen, W. Weber, and G. Radziwill (2013) “Optogenetic Control of Protein Kinase Activity in Mammalian Cells.” ACS Synth Biol Dx.doi.org/10.1021/sb400090s.
Yang, F., Y. Zhang, S. J. Ressler, M. M. Ittmann, G. E. Ayala, T. D. Dang, F. Wang and D. R. Rowley (2013). “FGFR1 is Essential for Prostate Cancer Progression and Metastasis.” Cancer Res. 73(12): 3716-24.
Ye, H., M. Daoud-El Baba, R. W. Peng and M. Fussenegger (2011). “A synthetic optogenetic transcription device enhances blood-glucose homeostasis in mice.” Science 332(6037): 1565-1568.
Zhao, M., D. Li, K. Shimazu, Y. X. Zhou, B. Lu and C. X. Deng (2007). “Fibroblast growth factor receptor-1 is required for long-term potentiation, memory consolidation, and neurogenesis.” Biol Psychiatry 62(5): 381-390.
Zoltowski, B. D. and K. H. Gardner (2011). “Tripping the light fantastic: blue-light photoreceptors as examples of environmentally modulated protein-protein interactions.” Biochemistry 50(1): 4-16.

1-45. (canceled)

46. A chimeric fusion protein, comprising a light sensing domain, wherein the chimeric fusion protein is capable of homodimerizing, when the light sensing domain is excited with light of a suitable wavelength; and wherein the chimeric fusion protein further comprises the intracellular part of a receptor tyrosine kinase (RTK),
wherein the light sensing domain is selected from
(i) a LOV domain with an amino acid sequence having at least 74% sequence identity to SEQ ID NO: 10 (VfAU1-LOV),
(ii) a LOV domain with an amino acid sequence having at least 76% sequence identity to SEQ ID NO: 12 (NgPA1-LOV),
(iii) a LOV domain with an amino acid sequence having at least 74% sequence identity to SEQ ID NO: 14 (OdPA1-LOV), or
(iv) an amino acid sequence with at least 70% sequence identity over the whole length to SEQ ID NO: 64 (SyCP1-PHY), in functional linkage with a chromophore; and
wherein the RTK is selected from the group consisting of FGF receptors, EGF receptors, RET receptors, and Trk receptors.

47. The chimeric fusion protein of claim 46 (ii), wherein the LOV domain has an amino acid sequence with at least 78% sequence identity over the whole length of the amino acid sequence of SEQ ID NO: 12 (NgPA1-LOV).

48. The chimeric fusion protein of claim 46 (iii), wherein the LOV domain has an amino acid sequence with at least 75% sequence identity over the whole length of the amino acid sequence of SEQ ID NO: 14 (OdPA1-LOV).

49. The chimeric fusion protein of claim 46 (i), wherein the LOV domain has an amino acid sequence with at least 73% sequence identity over the whole length of the amino acid sequence of SEQ ID NO: 10 (VfAU1-LOV).

50. The chimeric fusion protein of claim 46, wherein the light sensing domain is a LOV domain, capable of being activated at 5 μW/mm² of light.

51. The chimeric fusion protein of claim 46, wherein the light for activating the LOV domain has a wavelength in the range of 350-500 nm.

52. The chimeric fusion protein of claim 46 (iv), wherein the light sensing domain has an amino acid sequence with at least 78% sequence identity over the whole length to the amino acid sequence of SEQ ID NO: 64 (SyCP1-PHY).

53. The chimeric fusion protein of claim 46 (iv),
wherein the chimeric fusion protein has at least 70% sequence identity over the whole length of the amino acid sequence of SEQ ID NO: 66 (redOpto-mFGFR1), or
wherein the chimeric fusion protein has at least 70% sequence identity over the whole length of the amino acid sequence of SEQ ID NO: 67 (redOpto-rtrkB).

54. The chimeric fusion protein of claim 46 (iv), wherein the chromophore is a linear tetrapyrrole selected from phycocyanonbilin, phycoerythrobilin, phycourobilin, phycoviolobilin, phytochromobilin, biliverdin, bilirubin, mesobiliverdin, mesobilirubin, bilane, bilin, urobilin, stercobilin, and urobilinogen.

55. The chimeric fusion protein of claim 46 (iv), wherein the light sensing domain is capable of being activated at 0.5 μW/mm² of light.

56. The chimeric fusion protein of claim 46 (iv), wherein the light for activating the light sensing domain has a wavelength in the range of 600-690 nm.

57. The chimeric fusion protein of claim 46 (iv), wherein the light for inactivating the light sensing domain has a wavelength in the range of 700-750 nm.

58. The chimeric fusion protein of claim 46, wherein the light sensing domain is located at the C-terminus of the chimeric fusion protein.

59. The chimeric fusion protein of claim 46, wherein the light sensing domain is a LOV domain with an amino acid sequence having at least 74% sequence identity to SEQ ID NO: 10 (VfAU1-LOV), and wherein the RTK is selected from the group consisting of FGFR1, EGFR, and RET.

60. The chimeric fusion protein of claim 46, wherein the tyrosine kinase is a RTK selected from the group consisting of EGFR, FGFR1, RET, and TrkB receptors.

61. The chimeric fusion protein of claim 46, wherein the chimeric fusion protein further comprises a fluorescence protein.

62. A nucleic acid molecule encoding the chimeric fusion protein as defined in claim 46.

63. The nucleic acid molecule of claim 62, comprising the nucleic acid sequence of SEQ ID NO: 68 (redOpto-mFGFR1) or SEQ ID NO: 69 (redOpto-rtrkB).

64. A non-human transgenic animal, which expresses the chimeric fusion protein encoded by the nucleic acid molecule according to claim 62.

65. A research method, comprising the step of using a research tool selected from the chimeric fusion protein according to claim 46, the nucleic acid molecule according to claim 62, and the non-human transgenic animal according to claim 64.

66. A screening method comprising the step of providing a non-human transgenic animal according to claim 64, and using said animal in a screening method.

67. A non-therapeutic method for controlling cell growth, comprising the step of using the chimeric fusion protein according to claim 46 or the nucleic acid molecule according to claim 62 in a cell for controlling cell growth of said cell.

68. A method of producing patterned cell cultures, comprising the step of using the chimeric fusion protein according to claim 46 or the nucleic acid molecule according to claim 62 in cultured cells for producing patterned cell cultures.

69. A non-therapeutic method for controlling growth factor pathways, comprising the step of using the chimeric fusion protein according to claim 46 or the nucleic acid molecule according to claim 62 in a cell for controlling growth factor pathways in said cell.

70. A non-therapeutic method for controlling the production of a biologic product of interest, comprising the step of using the chimeric fusion protein according to claim 46 or the nucleic acid molecule according to claim 62 in a cell for controlling the production of a biologic product of interest in said cell.

71. A non-therapeutic method for differentiating stem cells, comprising the step of differentiating stem cells using the chimeric fusion protein according to claim 46 or the nucleic acid molecule according to claim 62, wherein the stem cell is not produced using a process which involves modifying the germ line genetic identity of human beings or which involves use of a human embryo for industrial or commercial purposes.

72. A screening method, comprising the steps of
a) providing a cell which expresses a chimeric fusion protein, comprising
a LOV domain having an amino acid sequence with at least 70% sequence identity over the whole length of an amino acid sequence selected from SEQ ID NO: 10 (VfAU1-LOV), SEQ ID NO: 12 (NgPA1-LOV), and SEQ ID NO: 14 (OdPA1-LOV), or a light sensing domain having an amino acid sequence with at least 70% sequence identity over the whole length to the amino acid sequence of SEQ ID NO: 64 (SyCP1-PHY), in functional linkage with a chromophore; and
the intracellular part of a receptor tyrosine kinase (RTK) selected from the group consisting of FGF receptors, EGF receptors, RET receptors, and Trk receptors;
wherein the chimeric fusion protein is capable of homodimerizing upon excitation of the LOV domain or light sensing domain with light of a suitable wavelength, thereby triggering a cell response via said intracellular part of said cell surface receptor;
b) contacting said cell with a candidate agent;
c) exposing said cell with said light of a suitable wavelength; and
d) determining whether said candidate agent is capable of affecting said cell response triggered in step c).

73. The method of claim 72, wherein the LOV domain has an amino acid sequence with at least 73% sequence identity over the whole length of the amino acid sequence of SEQ ID NO: 12 (NgPA1-LOV).

74. The method of claim 72, wherein the LOV domain has an amino acid sequence with at least 73% sequence identity over the whole length of the amino acid sequence of SEQ ID NO: 14 (OdPA1-LOV).

75. The method of claim 72, wherein the LOV domain has an amino acid sequence with at least 73% sequence identity over the whole length of the amino acid sequence of SEQ ID NO: 10 (VfAU1-LOV).

76. The method of claim 73, wherein the light for activating the LOV domain has a wavelength in the range of 350-500 nm.

77. The method of any one of claim 73, 74, or 75, wherein the LOV domain is capable of being activated at 5 μW/mm² of light.

78. The method of claim 72, wherein the light sensing domain has an amino acid sequence with at least 73% sequence identity over the whole length to the amino acid sequence of SEQ ID NO: 64 (SyCP1-PHY).

79. The method of claim 78, wherein the chromophore is a linear tetrapyrrole selected from phycocyanonbilin, phycoerythrobilin, phycourobilin, phycoviolobilin, phytochromobilin, biliverdin, bilirubin, mesobiliverdin, mesobilirubin, bilane, bilin, urobilin, stercobilin, and urobilinogen.

80. The method of claim 78, wherein the light for activating the light sensing domain has a wavelength in the range of 600-690 nm.

81. The method of claim 78, wherein the light for inactivating the light sensing domain has a wavelength in the range of 700-750 nm.

82. The method of claim 78, wherein the light sensing domain is capable of being activated at 0.5 μW/mm² of light.

83. The method of claim 72, wherein the LOV domain or light sensing domain is located at the C-terminus of the chimeric fusion protein.

84. The method of claim 72, wherein said fusion protein further comprises the transmembrane domain of said RTK.

85. The method of claim 73, wherein the tyrosine kinase is a RTK selected from the group consisting of EGFR, FGFR1, RET, and TrkB receptors.

86. The method of claim 73, wherein step d) uses light as the read-out of the change in the cell response.

87. The method of claim 73, wherein step d) comprises
(i) determination of the cell cycle distribution, and/or
(ii) determination of the gene transcriptional profile of the cell, and/or
(iii) determination of the localization of proteins in the cell, and/or
(iv) determination of the functional state of proteins in the cell, and/or
(v) determination of the shape of cells, and/or
(vi) determination of the distribution of cells on a surface or in 3D structure, and/or
(vii) determination of the migratory behavior of cells on a surface or in 3D structure, and/or
(viii) determination of the metabolic activity of cells, and/or
(ix) determination of the survival or death of cells, and/or
(x) determination of the differentiation state of cells, and/or
(xi) determination of the composition of metabolites of cells, and/or
(xii) determining the incorporation of a nucleotide analogue by the cell, preferably wherein the nucleotide analogue is 5-ethynyl-2′-deoxyuridine or bromodeoxyuridine, more preferably wherein the nucleotide analogue is fluorescent labelled or wherein the nucleotide analogues are detected by an antibody, most preferable wherein the fluorescent molecule are fluorescent azides.

88. The method of claim 73, wherein step d) comprises determination of the gene transcriptional profile of the cell, more preferably using a reporter gene assay, most preferably using a luciferase reporter gene assay.

89. The method of claim 73, wherein step d) comprises determining the incorporation of a fluorescent nucleotide analogue by the cell, preferably wherein the fluorescent nucleotide analogue is 5-ethynyl-2′-deoxyuridine.
